(19) United States
(12) Patent Application Publication
(10) Pub. No.: US 2022/0409629 A1
(43) Pub. Date: Dec. 29, 2022

(54) BENZODIAZEPINE DERIVATIVES FOR TREATING A RESPIRATORY SYNCYTIAL VIRUS (RSV) INFECTION

(71) Applicant: ReViral Limited, Stevenage (GB)

(72) Inventors: MATTHEW BARRETT, Stevenage (GB); GEORGE STUART COCKERILL, Stevenage (GB); JAMES GOOD, Stevenage (GB); CRAIG ALEX AVERY, Nottingham (GB); EDWARD JAMES COCHRANE, Nottingham (GB)

(21) Appl. No.: 17/770,455

(22) PCT Filed: Oct. 22, 2020
(86) PCT No.: PCT/GB2020/052658

§ 371 Date: Apr. 20, 2022

(30) Foreign Application Priority Data
Oct. 22, 2019 (GB) 1915273.5

(51) Int. Cl. A61K 31/5513; C07D 401/14; C07D 403/12; C07D 405/14; A61P 31/14; A61K 39/42; A61K 45/06; A61K 9/20; A61K 9/00; A61K 9/08; A61K 47/40

(52) U.S. Cl. CPC A61K 31/5513; C07D 401/14; C07D 403/12; C07D 405/14; A61P 31/14; A61K 39/42; A61K 45/06; A61K 9/2009; A61K 9/0019; A61K 9/0095; A61K 9/2013; A61K 9/2018; A61K 9/2059; A61K 9/08; A61K 47/40

(57) ABSTRACT

Benzodiazepine derivatives of formula (I): wherein: each of R¹ and R² is independently H or halo; R³ is H, C₁-C₆ alkyl, —NHR⁸ or —OR′; either (i) a, c, and e are all bonds, with b, d and f absent; orb, d and f are all bonds, with a, c and e absent; R⁴ is H or a group selected from C₁-C₆ alkyl, C₃-C₆ cycloalkyl and 4- to 10-membered heterocyclyl, the group being unsubstituted or substituted; R⁵ is H or halo; R⁶ is —OR⁸, —NR⁸R⁹ or —R⁸; R⁷ is H or halo; each of R⁸ and R⁹ is independently H or a group selected from C₁-C₆ alkyl, C₃-C₆ cycloalkyl and 4- to 10-membered heterocyclyl, the group being unsubstituted or substituted; R′ is H or C₁-C₆ alkyl; and one of V and W is CH and the other is N or CH; and the pharmaceutically acceptable salts thereof are inhibitors of RSV and can therefore be used to treat or prevent an RSV infection.

### FIELD OF THE INVENTION

The present invention relates to benzodiazepine derivatives and to their use in treating or preventing a respiratory syncytial virus (RSV) infection.

### BACKGROUND TO THE INVENTION

RSV is a negative-sense, single-stranded RNA virus of the Paramyxoviridae family. RSV is readily transmitted by secretions from an infected person via surfaces or hand-to-hand transfer. Unlike influenza, it is not transmitted by small-particle aerosols. Following successful inoculation, the incubation period is between four and six days during which time the virus spreads from the nasopharynx to the lower respiratory tract by fusion of infected with uninfected cells and by sloughing of the necrotic epithelium. In infants, coupled with increased mucus secretion and oedema, this can lead to mucus plugging causing hyper-inflation and collapse of distal lung tissue indicative of bronchiolitis. Hypoxia is common and the ability to feed is often impaired because of respiratory distress. In RSV pneumonia, inflammatory infiltration of the airways consists of mononuclear cells and is more generalised, with involvement of the bronchioles, bronchi and alveoli. The duration and degree of viral shedding has been found to correlate with the clinical signs and severity of disease.

RSV is the leading cause of serious respiratory tract infections in infants and young children throughout the world. The highest morbidity and mortality occurs in those born prematurely and for those with chronic lung or heart disease, although many infants hospitalised for RSV infection are otherwise healthy. Severe RSV infection in infancy can lead to several years of recurrent wheezing and is linked to the later development of asthma.

RSV is also a major cause of morbidity and mortality in the elderly and in immunocompromised children and adults as well as those with chronic obstructive pulmonary disease (COPD) and congestive heart failure (CHF).

RSV has a seasonal incidence; it is highly predictable and occurs in the winters of both hemispheres, from September to May in Europe and North America, peaking in December and January, and can occur throughout the year in tropical countries. It affects>90% of infants and young children by the age of two years and as natural immunity is short-lived; many will be re-infected each year. As with influenza, in elderly people, RSV causes around 10% of winter hospitalisations with an associated mortality of 10%.

Current anti-RSV treatment involves the use of a monoclonal antibody to RSV, called palivizumab. Such use of palivizumab is a prophylactic, rather than therapeutic, treatment of RSV. Although this antibody is often effective, its use is restricted to preterm infants and infants at high risk. Indeed, its limited utility means that it is unavailable for many people in need of anti-RSV treatment. There is therefore an urgent need for effective alternatives to existing anti-RSV treatment.

Small molecules have also been proposed as inhibitors of RSV. These include benzimidazoles and benzodiazepines. For instance, the discovery and initial development of RSV604, a benzodiazepine compound having sub-micromolar anti-RSV activity, is described in Antimicrobial Agents and Chemotherapy, September 2007, 3346-3353 (Chapman et al). Benzodiazepine inhibitors of RSV are also disclosed in publications including WO2004/026843 and WO2005/089770 (Arrow Therapeutics Limited); WO2016/166546 and WO2018/033714 (Durham University); and WO2017/015449, WO2018/129287 and WO2018/226801 (Enanta Pharmaceuticals, Inc.).

There exists a need to identify further compounds that have anti-RSV activity, in particular compounds having a combination of potent anti-viral activity and favourable pharmacokinetic properties.

### SUMMARY OF THE INVENTION

It has now been found that a novel series of benzodiazepine derivatives have potent anti-RSV activity with favourable pharmacokinetics and good solubility. Accordingly, the present invention provides a compound which is a benzodiazepinyl pyrazole of formula (I):

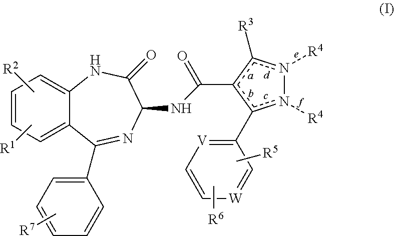

wherein:
each of R¹ and R² is independently H or halo;
R³ is H, C₁-C₆ alkyl, —NHR⁸ or —OR;
either (i) [symbol] , [symbol] and [symbol] are all bonds, with [symbol] , [symbol] and [symbol] absent; or [symbol] , [symbol] and [symbol] are all bonds, with [symbol] , [symbol] and [symbol] absent;
R⁴ is H or a group selected from C₁-C₆ alkyl, C₃-C₆ cycloalkyl and 4- to 10-membered heterocyclyl, the group being unsubstituted or substituted;
R⁵ is H or halo;
R⁶ is —OR⁸, —NR⁸R⁹ or —R⁸;
R⁷ is H or halo;
each of R⁸ and R⁹ is independently H or a group selected from C₁-C₆ alkyl, C₃-C₆ cycloalkyl and 4- to 10-membered heterocyclyl, the group being unsubstituted or substituted;
R′ is H or C₁-C₆ alkyl; and
one of V and W is CH and the other is CH or N;
or a pharmaceutically acceptable salt thereof.

### DETAILED DESCRIPTION OF THE INVENTION

When any group, ring, substituent or moiety defined herein is substituted, it is typically substituted by Q as defined below.

A C₁₋₆ alkyl group or moiety is linear or branched. A C₁₋₆ alkyl group is typically a C₁₋₄ alkyl group, or a C₄₋₆ alkyl group. Examples of C₁₋₆ alkyl groups and moieties include methyl, ethyl, n-propyl, i-propyl, n-butyl, i-butyl, t-butyl, n-pentyl, i-pentyl (i.e. 3-methylbut-1-yl), t-pentyl (i.e. 2-methylbut-2-yl), neopentyl (i.e. 2,2-dimethylpropan-1-yl), n-hexyl, i-hexyl (i.e. 4-methylpentan-1-yl), t-hexyl (i.e. 3-methylpentan-3-yl) and neopentyl (i.e. 3,3-dimethylbutan-1-yl). For the avoidance of doubt, where two alkyl moieties are present in a group, the alkyl moieties may be the same or different. A C₁₋₆ alkyl group is unsubstituted or substituted, typically by one or more groups Q as defined below. For example, a C₁₋₆ alkyl group is unsubstituted or substituted by 1, 2 or 3 groups Q as defined below.

Q is halo, nitro, —CN, OH, C₁₋₆ alkoxy, C₁₋₆ hydroxyalkyl, C₁₋₆ alkylthio, C₁₋₆ alkyl, C₁₋₆ haloalkyl, C₁₋₄ haloalkoxy, —CO₂R′, —NR′₂, —SR′, —S(═O)R′, —S(═O)₂R′, C₃-C₁₀ cycloalkyl, 5 to 10-membered heterocyclyl, 5- to 12-membered aryl or 5- to 10-membered heteroaryl, wherein each R′ is independently selected from H, C₁₋₆ alkyl, C₃₋₁₀ cycloalkyl, 5 to 10-membered heterocyclyl, C₆-C₁₀ aryl and 5- to 10-membered heteroaryl. For the avoidance of doubt, the alkyl, alkoxy, alkylthio, cycloalkyl, heterocyclyl, aryl and heteroaryl moieties in these definitions are themselves typically unsubstituted.

A C₁₋₆ alkoxy group is linear or branched. It is typically a C₁₋₄ alkoxy group, for example a methoxy, ethoxy, propoxy, i-propoxy, n-propoxy, n-butoxy, sec-butoxy or tert-butoxy group. A C₁₋₆ alkoxy group is unsubstituted or substituted, typically by one or more groups Q as defined above.

A C₁₋₆ alkylthio group is linear or branched. It is typically a C₁₋₄ alkylthio group, for example a methylthio, ethylthio, propylthio, i-propylthio, n-propylthio, n-butylthio, sec-butylthio or tert-butylthio group. A C₁₋₆ alkylthio group is unsubstituted or substituted, typically by one or more groups Q as defined above.

A halogen or halo group is F, Cl, Br or I. Typically it is F or Cl. A C₁₋₆ alkyl group substituted by halogen may be denoted “C₁₋₆ haloalkyl”, which means a C₁₋₆ alkyl group as defined above in which one or more hydrogens is replaced by halo. Likewise a C₁₋₆ alkoxy group substituted by halogen may be denoted “C₁₋₆ haloalkoxy”, which means a C₁₋₆ alkoxy group as defined above in which one or more hydrogens is replaced by halo. Typically, C₁₋₆ haloalkyl or C₁₋₆ haloalkoxy is substituted by 1, 2 or 3 said halogen atoms. Haloalkyl and haloalkoxy groups include perhaloalkyl and perhaloalkoxy groups such as —CX₃ and —OCX₃ wherein X is a halogen, for example —CF₃—CCl₃—OCF₃ and —OCCl₃.

A C₁₋₆ hydroxyalkyl group is a C₁₋₆ alkyl group as defined above, substituted by one or more OH groups. Typically, it is substituted by one, two or three OH groups. Preferably, it is substituted by a single OH group.

A C₆-C₁₀ aryl group is an aromatic carbocyclic group containing from 6 to 10 carbon atoms. It is monocyclic or a fused bicyclic ring system in which an aromatic ring is fused to another aromatic carbocyclic ring. Examples of a C₆-C₁₀ aryl group include phenyl and naphthyl. When substituted, an aryl group is typically substituted by a group Q as defined above, for instance by 1, 2 or 3, groups selected from a group Q as defined above. More particularly, a substituted aryl group such as a substituted phenyl group is substituted by 1 or 2 groups selected from C₁-C₆ alkyl, halo, —OR⁸ and —N(R⁸)₂ wherein R⁸ is H or C₁-C₆ alkyl, each R⁸ being the same or different when two are present.

A C₃₋₁₀ cycloalkyl group is a saturated hydrocarbon ring having from 3 to 10 carbon atoms. A C₃₋₁₀ cycloalkyl group may be, for instance, C₃-C₇ cycloalkyl such as cyclopropyl, cyclobutyl, cyclopentyl, cyclohexyl, or cycloheptyl. Typically it is C₃-C₆ cycloalkyl, or C₄-C₆ cycloalkyl, for example cyclobutyl, cyclopentyl or cyclohexyl. In one embodiment it is cyclobutyl. A C₃₋₁₀ cycloalkyl group is unsubstituted or substituted, typically by one or more groups Q as defined above.

A 4- to 10-membered heteroaryl group or moiety is a 4- to 10-membered aromatic heterocyclic group which contains 1, 2, 3, or 4 heteroatoms selected from O, N and S. It is monocyclic or bicyclic. Typically it contains one N atom and 0, 1, 2 or 3 additional heteroatoms selected from O, S and N. It may be, for example, a monocyclic 5- to 7-membered heteroaryl group, for instance a 5- or 6-membered N-containing heteroaryl group. Examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl and pyrazolyl groups. Furanyl, thienyl, imidazolyl, pyridyl and pyrimidyl groups are preferred. It may alternatively be a bicyclic heteroaryl group, for instance an 8- to 10-membered bicyclic heteroaryl group. Examples include quinolyl, isoquinolyl, quinazolyl, quinoxalinyl, indolyl, isoindolyl, indazolyl, imidazopyridazinyl, pyrrolopyridinyl, pyrazolopyrimidinyl and pyrrolopyrimidinyl. When substituted, a heteroaryl group (monocyclic or bicyclic) is typically substituted by one or more, e.g. 1, 2 or 3, groups selected from C₁₋₄ alkyl and a group Q as defined above.

A 4- to 10-membered heterocyclyl group is a monocyclic or bicyclic non-aromatic, saturated or unsaturated ring system containing 5 to 10 carbon atoms and at least one atom or group selected from N, O, S, SO, SO₂ and CO, more typically N or O. When the ring system is bicyclic, one ring may be saturated and one ring unsaturated. Typically, it is a C₄₋₁₀ ring system in which 1, 2 or 3 of the carbon atoms in the ring are replaced with an atom or group selected from O, S, SO₂, CO and NH. More typically it is a monocyclic ring, preferably a monocyclic C₄-C₆ ring. Examples include piperidyl, piperidin-2,6-dionyl, piperidin-2-onyl, piperazinyl, morpholinyl, thiomorpholinyl, S,S-dioxothiomorpholinyl, 1,3-dioxolanyl, pyrrolidinyl, imidazol-2-onyl, pyrrolidin-2-onyl, oxetanyl, tetrahydrofuranyl and tetrahydropyranyl moieties.

For the avoidance of doubt, although the above definitions of heteroaryl and heterocyclyl groups refer to an “N” atom which can be present in the ring, it will be evident to a skilled chemist that any such N atom will be protonated (or will carry a substituent as defined above) if it is attached to each of its adjacent ring atoms via a single bond. Such protonated forms are embraced within the present definitions of heteroaryl and heterocyclyl groups.

In one embodiment of formula (I) as defined above R² is a halo substituent, in particular F, at the 9-position of the benzodiazepinyl ring system. Examples of such compounds are those of the following formula (I′):

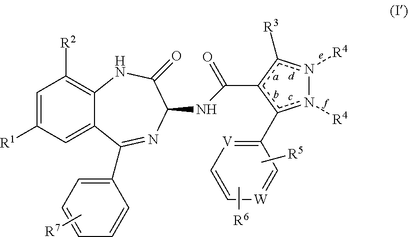

wherein R¹ is H or halo, R² is H or halo and the remaining groups and variables are as defined above for formula (I). Typically R¹ is H or F and R² is H or F. For instance, R¹ is H or F and R² is F.

In one embodiment of formulae (I), [symbol] , [symbol] and [symbol] are all bonds, with [symbol] , [symbol] and [symbol] absent Such compounds have the following formula (Ia):

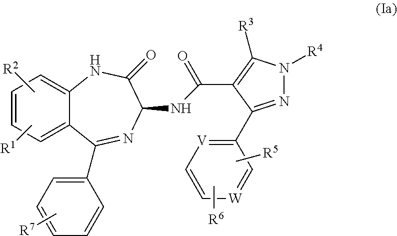

in which all the groups and variables are as defined above for formula (I) or (I′).

In another embodiment of formula (I), [symbol] , [symbol] and [symbol] are all bonds, with [symbol] , [symbol] and [symbol] absent. Such compounds have the following formula (Ib):

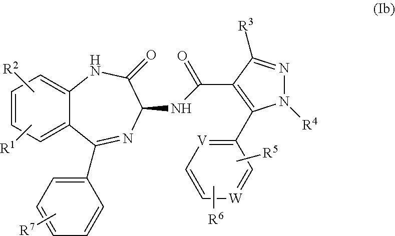

in which all the groups and variables are as defined above for formula (I) or (I′).

In one embodiment of the above formulae (I), (I′), (Ia) and (Ib), V is N and W is CH. Examples of such structures include benzodiazepinyl pyrazoles of the following formulae (Ia′) and (Ib′):

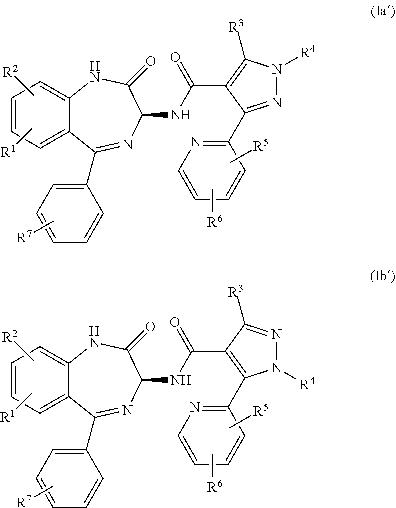

In formulae (Ia′) and (Ib′), each of R¹ to R⁷ is as defined above for formula (I) or (I′).

In another embodiment of the above formulae (I), (I′), (Ia) and (Ib), V is CH and W is N. Examples of such structures include benzodiazepinyl pyrazoles of the following formulae (Ia″) and (Ib″):

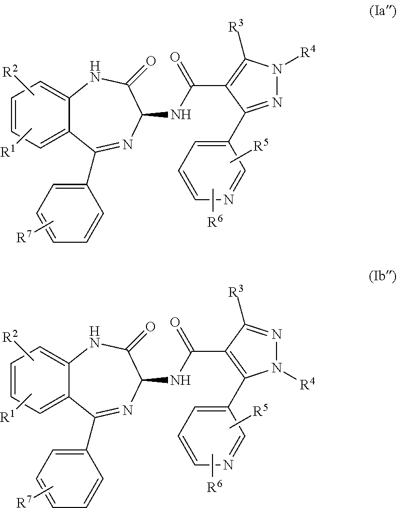

In formulae (Ia″) and (Ib″), each of R¹ to R⁷ is as defined above for formula (I) or (I′).

In a further embodiment of the above formulae (I), (I′), (Ia) and (Ib), V is CH and W is CH. Examples of such structures include benzodiazepinyl pyrazoles of the following formulae (Ia′″) and (Ib′″)

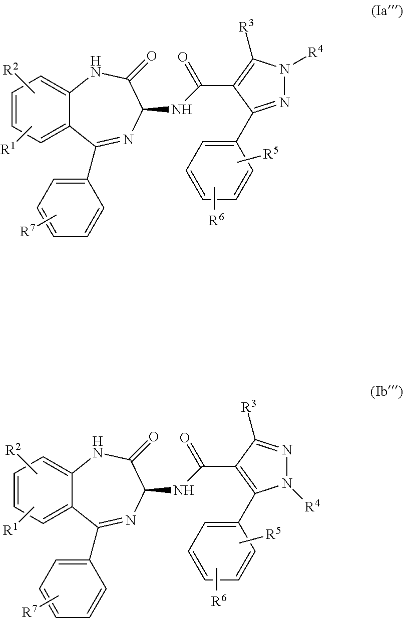

In formulae (Ia′″) and (Ib′″), each of R¹ to R⁷ is as defined above for formula (I) or (I′).

In compounds of the invention having any of the structural formulae defined above, R⁵ may be bonded at any available ring position of the six-membered ring to which it is attached. In one embodiment it is bonded at ring position 2, i.e. ortho to the bond that links the six-membered ring to the adjacent pyrazole ring. Typically R⁵ is F at the 2-position, i.e. a 2-fluoro group.

In compounds of the invention having any of the structural formulae defined above, R⁶ may be bonded at any available ring position of the six-membered ring to which it is attached. In one embodiment it is bonded at ring position 4, i.e. para to the bond that links the six-membered ring to the adjacent pyrazole ring.

In one aspect, the invention provides a compound which is a benzodiazepinyl pyrazole of the following formula (I″):

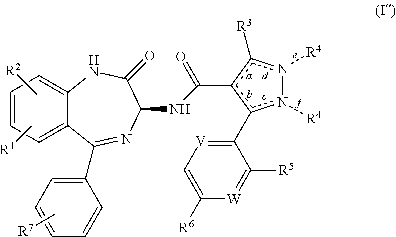

wherein each of the groups and variables is as defined above for formula (I), or a pharmaceutically acceptable salt thereof.

When R¹ and R² in formula (I″) take the same ring positions shown in formula (I′) above, the resulting compound is a benzodiazepinyl pyrazole of the following formula (I′″):

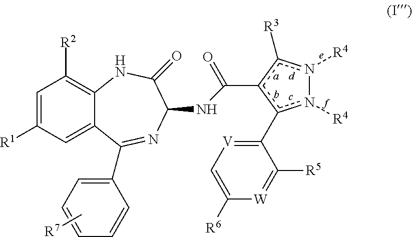

wherein R¹ is H or halo, R² is H or halo and the remaining groups and variables are as defined above for formula (I). Typically R¹ is H or F and R² is H or F. For instance, R¹ is H and R² is F.

In one embodiment of compounds of the invention having any of the structural formulae (I″), (Ia), (Ib), (Ia′), (Ib′), (Ia″), (Ib″), (Ia′″) or (Ib′″) as defined above, R² is at the 9-position of the benzodiazepinyl ring system. In this embodiment, typically R² is a halo substituent, in particular F. More typically in this embodiment, R¹ is H or F and R² is H or F. For instance, R¹ is H or F and R² is F.

In compounds of the invention having any of the structural formulae defined above, R³ is a group selected from H, C₁-C₆ alkyl, —NR⁸R⁹ and —OR′, wherein R′ is H or C₁-C₆ alkyl, for instance methyl or ethyl, and each of R⁸ and R⁹ is independently H or a group selected from C₁-C₆ alkyl, C₃-C₆ cycloalkyl and 4- to 10-membered heterocyclyl, the group being unsubstituted or substituted. In one embodiment of the structural formulae defined above, R³ is a group selected from H, C₁-C₆ alkyl and —NR⁸R⁹. Typically R⁸ is H and R⁹ is H or a group selected from C₁-C₆ alkyl, C₃-C₆ cycloalkyl and 4- to 10-membered heterocyclyl, the group being unsubstituted or substituted. In one embodiment R⁸ is H and R⁹ is H or C₁-C₆ alkyl.

In compounds of the invention having any of the structural formulae defined above, R⁴ is H or a group selected from C₁-C₆ alkyl, C₃-C₆ cycloalkyl and 4- to 10-membered heterocyclyl, the group being unsubstituted or substituted. In one embodiment R⁴ is a group selected from C₁-C₆ alkyl, C₃-C₆ cycloalkyl and 4- to 10-membered heterocyclyl, the group being unsubstituted or substituted. Typically R⁴ is a group selected from C₁-C₆ alkyl (such as C₁-C₃ alkyl), C₃-C₆ cycloalkyl (such as cyclopropyl) and 4- to 10-membered heterocyclyl (for instance, an O-containing heterocyclyl group such as oxetanyl, tetrahydrofuranyl or tetrahydropyranyl).

In compounds of the invention having any of the structural formulae defined above, R⁵ is H or halo, in particular F.

In compounds of the invention having any of the structural formulae defined above, R⁶ is —OR⁸, —NR⁸R⁹ or —R⁸ wherein each of R⁸ and R⁹ is H or a group selected from C₁-C₆ alkyl, C₃-C₆ cycloalkyl and 4- to 10-membered heterocyclyl, the group being unsubstituted or substituted. Typically R⁶ is selected from —OR⁸, —NR⁸R⁹ and R⁸, wherein R⁸ is C₁-C₆ alkyl (such as C₁-C₃ alkyl), C₃-C₆ cycloalkyl (such as cyclopropyl or cyclobutyl) and R⁹ is H or C₁-C₆ alkyl, the alkyl and cycloalkyl groups being unsubstituted or substituted. More typically R⁶ is —OR⁸, —NR⁸R⁹ or R⁸, for instance —OR⁸ or —NR⁸R⁹, wherein R⁸ is unsubstituted C₁-C₆ alkyl (such as methyl, ethyl or i-propyl) or C₃-C₆ cycloalkyl (such as cyclopropyl or cyclobutyl), the cycloalkyl group being unsubstituted or substituted by unsubstituted C₁-C₃ alkyl (such as methyl), and R⁹ is C₁-C₆ alkyl or H.

Specific compounds of the invention include the following:
1-Ethyl-5-[2-fluoro-6-(methylamino)pyridin-3-yl]-N-[(3S)-2-oxo-5-phenyl-1,3-dihydro-1,4-benzodiazepin-3-yl]pyrazole-4-carboxamide;
1-Ethyl-5-[6-(ethylamino)-2-fluoropyridin-3-yl]-N-[(3S)-2-oxo-5-phenyl-1,3-dihydro-1,4-benzodiazepin-3-yl]pyrazole-4-carboxamide;
1-Ethyl-5-[2-fluoro-6-(propan-2-ylamino)pyridin-3-yl]-N-[(3S)-2-oxo-5-phenyl-1,3-dihydro-1,4-benzodiazepin-3-yl]pyrazole-4-carboxamide;
1-Ethyl-5-[2-fluoro-6-(cyclopropylamino)pyridin-3-yl]-N-[(3S)-2-oxo-5-phenyl-1,3-dihydro-1,4-benzodiazepin-3-yl]pyrazole-4-carboxamide;
5-[6-(Cyclobutylamino)-2-fluoropyridin-3-yl]-1-ethyl-N-[(3S)-2-oxo-5-phenyl-1,3-dihydro-1,4-benzodiazepin-3-yl]pyrazole-4-carboxamide;
1-Ethyl-5-[2-fluoro-6-[(1-methylcyclopropyl)amino]pyridin-3-yl]-N-[(3S)-2-oxo-5-phenyl-1,3-dihydro-1,4-benzodiazepin-3-yl]pyrazole-4-carboxamide;
5-[5-(Cyclopropylamino)-3-fluoropyridin-2-yl]-1-ethyl-N-[(3S)-2-oxo-5-phenyl-1,3-dihydro-1,4-benzodiazepin-3-yl]pyrazole-4-carboxamide;
1-Ethyl-5-[3-fluoro-5-(propan-2-ylamino)pyridin-2-yl]-N-[(3S)-2-oxo-5-phenyl-1,3-dihydro-1,4-benzodiazepin-3-yl]pyrazole-4-carboxamide;
1-Ethyl-5-[2-fluoro-4-(propan-2-ylamino)phenyl]-N-[(3S)-2-oxo-5-phenyl-1,3-dihydro-1,4-benzodiazepin-3-yl]pyrazole-4-carboxamide;
1-Ethyl-5-[6-(ethylamino)-2-fluoropyridin-3-yl]-3-methyl-N-[(3S)-2-oxo-5-phenyl-1,3-dihydro-1,4-benzodiazepin-3-yl]pyrazole-4-carboxamide;
5-[6-(Cyclopropylamino)-2-fluoropyridin-3-yl]-1-ethyl-N-[(3S)-9-fluoro-2-oxo-5-phenyl-1,3-dihydro-1,4-benzodiazepin-3-yl]pyrazole-4-carboxamide;
5-[5-(Cyclopropylamino)-3-fluoropyridin-2-yl]-1-ethyl-N-[(3S)-9-fluoro-2-oxo-5-phenyl-1,3-dihydro-1,4-benzodiazepin-3-yl]pyrazole-4-carboxamide;
1-Ethyl-5-[5-(ethylamino)-3-fluoropyridin-2-yl]-N-[(3S)-9-fluoro-2-oxo-5-phenyl-1,3-dihydro-1,4-benzodiazepin-3-yl]pyrazole-4-carboxamide;
1-cyclopropyl-5-[6-(ethylamino)-2-fluoropyridin-3-yl]-N-[(3S)-2-oxo-5-phenyl-1,3-dihydro-1,4-benzodiazepin-3-yl]pyrazole-4-carboxamide;
1-cyclopropyl-5-[6-(cyclopropylamino)-2-fluoropyridin-3-yl]-N-[(3S)-2-oxo-5-phenyl-1,3-dihydro-1,4-benzodiazepin-3-yl]pyrazole-4-carboxamide;
5-[6-(cyclopropylamino)-2-fluoropyridin-3-yl]-N-[(3S)-2-oxo-5-phenyl-1,3-dihydro-1,4-benzodiazepin-3-yl]-1-propan-2-ylpyrazole-4-carboxamide;
1-Ethyl-N-[(3S)-9-fluoro-2-oxo-5-phenyl-1,3-dihydro-1,4-benzodiazepin-3-yl]-5-[3-fluoro-5-(propan-2-ylamino)pyridin-2-yl]pyrazole-4-carboxamide;
5-[6-(Cyclopropylamino)-2-fluoropyridin-3-yl]-1-(oxetan-3-yl)-N-[(3S)-2-oxo-5-phenyl-1,3-dihydro-1,4-benzodiazepin-3-yl]pyrazole-4-carboxamide;
5-[6-(Cyclopropylamino)-2-fluoropyridin-3-yl]-1-(oxetan-3-yl)-N-[(3S)-9-fluoro-2-oxo-5-phenyl-1,3-dihydro-1,4-benzodiazepin-3-yl]pyrazole-4-carboxamide;
5-[6-(Cyclopropylamino)-2-fluoropyridin-3-yl]-N-[(3S)-2-oxo-5-phenyl-1,3-dihydro-1,4-benzodiazepin-3-yl]-1-(oxan-4-yl)pyrazole-4-carboxamide;
5-[6-(Cyclopropylamino)-2-fluoropyridin-3-yl]-N-[(3S)-9-fluoro-2-oxo-5-phenyl-1,3-dihydro-1,4-benzodiazepin-3-yl]-1-(oxan-4-yl)pyrazole-4-carboxamide;
N-[(3S)-9-fluoro-2-oxo-5-phenyl-1,3-dihydro-1,4-benzodiazepin-3-yl]-5-[2-fluoro-6-(propan-2-ylamino)pyridin-3-yl]-1-(oxan-4-yl)pyrazole-4-carboxamide;
5-[2-Fluoro-6-(propan-2-ylamino)pyridin-3-yl]-1-(oxan-4-yl)-N-[(3S)-2-oxo-5-phenyl-1,3-dihydro-1,4-benzodiazepin-3-yl]pyrazole-4-carboxamide;
5-[6-(Ethylamino)-2-fluoropyridin-3-yl]-N-[(3S)-9-fluoro-2-oxo-5-phenyl-1,3-dihydro-1,4-benzodiazepin-3-yl]-1-(oxan-4-yl)pyrazole-4-carboxamide;
3-[6-(Cyclopropylamino)-2-fluoropyridin-3-yl]-1-ethyl-N-[(3S)-2-oxo-5-phenyl-1,3-dihydro-1,4-benzodiazepin-3-yl]pyrazole-4-carboxamide;
3-[6-(Cyclopropylamino)-2-fluoropyridin-3-yl]-1-ethyl-N-[(3S)-9-fluoro-2-oxo-5-phenyl-1,3-dihydro-1,4-benzodiazepin-3-yl]pyrazole-4-carboxamide;
1-Ethyl-3-[2-fluoro-6-(propylamino)pyridin-3-yl]-N-[(3S)-2-oxo-5-phenyl-1,3-dihydro-1,4-benzodiazepin-3-yl]pyrazole-4-carboxamide;
1-Ethyl-N-[(3S)-9-fluoro-2-oxo-5-phenyl-1,3-dihydro-1,4-benzodiazepin-3-yl]-3-[2-fluoro-6-(propylamino)pyridin-3-yl]pyrazole-4-carboxamide;
1-tert-butyl-3-[6-(cyclopropylamino)-2-fluoropyridin-3-yl]-N-[(3S)-2-oxo-5-phenyl-1,3-dihydro-1,4-benzodiazepin-3-yl]pyrazole-4-carboxamide;
1-tert-butyl-3-[6-(cyclopropylamino)-2-fluoropyridin-3-yl]-N-[(3S)-9-fluoro-2-oxo-5-phenyl-1,3-dihydro-1,4-benzodiazepin-3-yl]pyrazole-4-carboxamide;
3-[6-(Cyclopropylamino)-2-fluoropyridin-3-yl]-1-(oxan-4-yl)-N-[(3S)-2-oxo-5-phenyl-1,3-dihydro-1,4-benzodiazepin-3-yl]pyrazole-4-carboxamide;
3-[6-(Cyclopropylamino)-2-fluoropyridin-3-yl]-N-[(3S)-9-fluoro-2-oxo-5-phenyl-2,3-dihydro-1H-1,4-benzodiazepin-3-yl]-1-(oxan-4-yl)-1H-pyrazole-4-carboxamide;
3-[6-(Cyclopropylamino)-2-fluoropyridin-3-yl]-N-[(3S)-9-fluoro-2-oxo-5-phenyl-2,3-dihydro-1H-1,4-benzodiazepin-3-yl]-1-(2,2,2-trifluoroethyl)-1H-pyrazole-4-carboxamide;
(3-[6-(Cyclopropylamino)-2-fluoropyridin-3-yl]-1-(4,4-difluorocyclohexyl)-N-[(3S)-2-oxo-5-phenyl-1,3-dihydro-1,4-benzodiazepin-3-yl]pyrazole-4-carboxamide;
3-[6-(Cyclopropylamino)-2-fluoropyridin-3-yl]-1-(4,4-difluorocyclohexyl)-N-[(3S)-9-fluoro-2-oxo-5-phenyl-1,3-dihydro-1,4-benzodiazepin-3-yl]pyrazole-4-carboxamide;
3-[6-(Cyclopropylamino)-2-fluoropyridin-3-yl]-N-[(3S)-9-fluoro-2-oxo-5-phenyl-1,3-dihydro-1,4-benzodiazepin-3-yl]-1-(oxetan-3-yl)pyrazole-4-carboxamide;
3-[6-(Cyclopropylamino)-2-fluoropyridin-3-yl]-1-(oxetan-3-yl)-N-[(3S)-2-oxo-5-phenyl-1,3-dihydro-1,4-benzodiazepin-3-yl]pyrazole-4-carboxamide;
-Ethyl-5-(3-fluoro-5-(methylamino)pyridin-2-yl)-N—((S)-2-oxo-5-phenyl-2,3-dihydro-1H-benzo[e][1,4]diazepin-3-yl)-1H-pyrazole-4-carboxamide;
1-Ethyl-5-(5-(ethylamino)-3-fluoropyridin-2-yl)-N—((S)-2-oxo-5-phenyl-2,3-dihydro-1H-benzo[e][1,4]diazepin-3-yl)-1H-pyrazole-4-carboxamide;
5-[6-(Ethylamino)-2-fluoropyridin-3-yl]-N-[(3S)-9-fluoro-2-oxo-5-phenyl-1,3-dihydro-1,4-benzodiazepin-3-yl]-1-(oxetan-3-yl)pyrazole-4-carboxamide;
N-[(3S)-9-Fluoro-2-oxo-5-phenyl-1,3-dihydro-1,4-benzodiazepin-3-yl]-5-[2-fluoro-6-(propan-2-ylamino)pyridin-3-yl]-1-(oxetan-3-yl)pyrazole-4-carboxamide;
5-[6-(Ethylamino)-2-fluoropyridin-3-yl]-1-(oxan-4-yl)-N-[(3S)-2-oxo-5-phenyl-1,3-dihydro-1,4-benzodiazepin-3-yl]pyrazole-4-carboxamide;
5-(4-Cyclopropyl-2-fluorophenyl)-1-(oxan-4-yl)-N-[(3S)-2-oxo-5-phenyl-1,3-dihydro-1,4-benzodiazepin-3-yl]pyrazole-4-carboxamide;
5-(4-Cyclopropyl-2-fluorophenyl)-N-[(3S)-9-fluoro-2-oxo-5-phenyl-1,3-dihydro-1,4-benzodiazepin-3-yl]-1-(oxan-4-yl)pyrazole-4-carboxamide;
5-(2-Fluoro-4-propan-2-ylphenyl)-1-(oxan-4-yl)-N-[(3S)-9-fluoro-2-oxo-5-phenyl-1,3-dihydro-1,4-benzodiazepin-3-yl]pyrazole-4-carboxamide;
5-(4-Ethyl-2-fluorophenyl)-1-(oxan-4-yl)-N-[(3S)-2-oxo-5-phenyl-1,3-dihydro-1,4-benzodiazepin-3-yl]pyrazole-4-carboxamide;
5-(4-Ethyl-2-fluorophenyl)-1-(oxan-4-yl)-N-[(3S)-9-fluoro-2-oxo-5-phenyl-1,3-dihydro-1,4-benzodiazepin-3-yl]pyrazole-4-carboxamide;
5-(5-Cyclopropyl-2-fluoropyridin-3-yl)-1-ethyl-N-[(3S)-9-fluoro-2-oxo-5-phenyl-1,3-dihydro-1,4-benzodiazepin-3-yl]pyrazole-4-carboxamide;
5-(6-Cyclopropyl-2-fluoropyridin-3-yl)-N-[(3S)-9-fluoro-2-oxo-5-phenyl-1,3-dihydro-1,4-benzodiazepin-3-yl]-1-(oxan-4-yl)pyrazole-4-carboxamide;
5-(5-Cyclopropyl-2-fluoropyridin-3-yl)-1-(oxan-4-yl)-N-[(3S)-2-oxo-5-phenyl-1,3-dihydro-1,4-benzodiazepin-3-yl]pyrazole-4-carboxamide;
5-(5-Cyclopropyl-2-fluoropyridin-3-yl)-N-[(3S)-9-fluoro-2-oxo-5-phenyl-1,3-dihydro-1,4-benzodiazepin-3-yl]-1-(oxan-4-yl)pyrazole-4-carboxamide;
5-(6-Ethyl-2-fluoropyridin-3-yl)-1-(oxan-4-yl)-N-[(3S)-2-oxo-5-phenyl-1,3-dihydro-1,4-benzodiazepin-3-yl]pyrazole-4-carboxamide;
5-(6-Ethyl-2-fluoropyridin-3-yl)-1-(oxan-4-yl)-N-[(3S)-9-fluoro-2-oxo-5-phenyl-1,3-dihydro-1,4-benzodiazepin-3-yl]pyrazole-4-carboxamide;
3-Ethoxy-1-ethyl-5-(2-fluorophenyl)-N-[(3S)-2-oxo-5-phenyl-1,3-dihydro-1,4-benzodiazepin-3-yl]pyrazole-4-carboxamide;
3-Ethoxy-1-ethyl-N-[(3S)-9-fluoro-2-oxo-5-phenyl-1,3-dihydro-1,4-benzodiazepin-3-yl]-5-(2-fluorophenyl)pyrazole-4-carboxamide;
5-[6-(Cyclopropylamino)pyridin-3-yl]-1-(oxan-4-yl)-N-[(3S)-9-fluoro-2-oxo-5-phenyl-1,3-dihydro-1,4-benzodiazepin-3-yl]pyrazole-4-carboxamide;
5-[6-(Cyclopropylamino)-2-fluoropyridin-3-yl]-N-[(3S)-9-fluoro-2-oxo-5-phenyl-1,3-dihydro-1,4-benzodiazepin-3-yl]-1-(1-methylpiperidin-4-yl)pyrazole-4-carboxamide;
1-(Oxan-4-yl)-5-[6-(propan-2-ylamino)pyridin-3-yl]-N-[(3S)-9-fluoro-2-oxo-5-phenyl-1,3-dihydro-1,4-benzodiazepin-3-yl]pyrazole-4-carboxamide;
5-(4-Ethyl-2-fluorophenyl)-1-(1-methylpiperidin-4-yl)-N-[(3S)-9-fluoro-2-oxo-5-phenyl-1,3-dihydro-1,4-benzodiazepin-3-yl]pyrazole-4-carboxamide;
   and the pharmaceutically acceptable salts thereof.

The compounds of the invention may contain asymmetric or chiral centres, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Compounds of Formula (I) containing one or more chiral centre may be used in enantiomerically or diastereoisomerically pure form, or in the form of a mixture of isomers.

The present invention embraces all geometric and positional isomers of compounds of the invention as defined above. For example, if a compound of the invention incorporates a double bond or a fused ring, the cis- and trans-forms, as well as mixtures thereof, are embraced within the scope of the invention. Both the single positional isomers and mixture of positional isomers are also within the scope of the present invention.

The compounds of the present invention may exist in unsolvated as well as solvated forms with pharmaceutically acceptable solvents such as water, ethanol, and the like, and it is intended that the invention embrace both solvated and unsolvated forms.

The compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol tautomerizations. Valence tautomers include interconversions by reorganization of some of the bonding electrons.

Compounds of the invention can be prepared by the synthetic methods described in the Examples that follow, or by analogy with such methods using appropriate starting materials and methodologies familiar to the skilled chemist.

A benzodiazepine derivative of formula (I) can be converted into a pharmaceutically acceptable salt thereof, and a salt can be converted into the free compound, by conventional methods. For instance, a benzodiazepine derivative of formula (I) can be contacted with a pharmaceutically acceptable acid to form a pharmaceutically acceptable salt. A pharmaceutically acceptable salt is a salt with a pharmaceutically acceptable acid or base.

Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

Compounds of the present invention have been found in biological tests to be inhibitors of respiratory syncytial virus (RSV). They possess a combination of potent anti-RSV activity with good bioavailability and good solubility characteristics. This combination of properties makes the compounds therapeutically useful and superior as drug candidates to many compounds disclosed in the prior art references discussed earlier.

Accordingly, the present invention further provides a compound which is a benzodiazepine derivative of formula (I), as defined above, or a pharmaceutically acceptable salt thereof, for use in a method of treating the human or animal body by therapy.

The invention also provides a compound of the invention as defined above for use in a method treating or preventing an RSV infection. Still further, the present invention provides the use of a compound of the invention as defined above in the manufacture of a medicament for use in treating or preventing an RSV infection. A subject suffering from or susceptible to an RSV infection may thus be treated by a method comprising the administration thereto of a compound of the invention as defined above. The condition of the subject may thereby be improved or ameliorated.

The RSV infection is typically a respiratory tract infection. The RSV infection may be an infection in a child, for instance a child under ten years of age or an infant under two years of age. In one embodiment the invention provides a compound as defined above for use in treating or preventing an RSV infection in paediatric patients. Alternatively the infection may be an infection in a mature or elderly adult, for instance an adult over 60 years of age, an adult over 70 years of age, or an adult over 80 years of age. The invention further provides a compound for use in treating or preventing an RSV infection in geriatric patients.

The RSV infection may be an infection in an immunocompromised individual or an individual suffering from COPD or CIF. In another embodiment, the RSV infection is an infection in a non-compromised individual, for instance an individual who is otherwise healthy.

A compound of the present invention can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection, infusion, or by inhalation or nebulisation. The compound is preferably given by oral administration.

The dosage depends on a variety of factors including the age, weight and condition of the patient and the route of administration. Daily dosages can vary within wide limits and will be adjusted to the individual requirements in each particular. Typically, however, the dosage adopted for each route of administration when a compound is administered alone to adult humans is 0.0001 to 650 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

A unit dose form such as a tablet or a capsule will usually contain 1-250 mg of active ingredient. For example, a compound of formula (I) could be administered to a human patient at a dose of between 100-250 mg either once a day, twice or three times a day. For example, a compound of formula (I) could be administered to a human patient at a dose of between 100-250 mg either once a day, twice or three times a day.

The compounds of formula (I) and pharmaceutically acceptable salts thereof may be used on their own. Alternatively, they may be administered in the form of a pharmaceutical composition. The present invention therefore also provides a pharmaceutical composition comprising a compound of formula (I) or a pharmaceutically acceptable salt thereof as hereinbefore defined, in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The compounds of the invention may be administered in a variety of dosage forms. Thus, they can be administered orally, for example as tablets, troches, lozenges, aqueous or oily suspensions, solutions, dispersible powders or granules. The compounds of the invention may also be administered parenterally, whether subcutaneously, intravenously, intramuscularly, intrasternally, transdermally, by infusion techniques or by inhalation or nebulisation. The compounds may also be administered as suppositories.

Solid oral forms of the pharmaceutical composition of the invention may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulfates; and, in general, non-toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride. Further suitable carriers for suspensions include sterile water, hydroxypropylmethyl cellulose (HPMC), polysorbate 80, polyvinylpyrrolidone (PVP), aerosol AOT (i.e. sodium 1,2-bis(2-ethylhexoxycarbonyl)ethanesulphonate), pluronic F127 and/or captisol (i.e. sulfobutylether-beta-cyclodextrin).

The compounds of the invention may, for example, be formulated as aqueous suspensions in a carrier selected from:

(i) 0.5% w/v hydroxypropylmethyl cellulose (HPMC)/0.1% w/v polysorbate 80;
(ii) 0.67% w/v polyvinylpyrrolidone (PVP)/0.33% w/v aerosol AOT (sodium 1,2-bis(2-ethylhexoxycarbonyl)ethanesulphonate);
(iii) 1% w/v pluronic F 127; and
(iv) 0.5% w/v polysorbate 80.

The carriers may be prepared by standard procedures known to those of skill in the art. For example, each of the carriers (i) to (iv) may be prepared by weighing the required amount of excipient into a suitable vessel, adding approximately 80% of the final volume of water and magnetically stirring until a solution is formed. The carrier is then made up to volume with water. The aqueous suspensions of compounds of formula I may be prepared by weighing the required amount of a compound of formula I into a suitable vessel, adding 100% of the required volume of carrier and magnetically stirring.

Solutions for injection or infusion may contain as carrier, for example, sterile water or preferably they may be in the form of sterile, aqueous, isotonic saline solutions.

The compounds of the invention may also be administered in conjunction with other compounds used for the treatment of viral infections. Thus, the invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment or prevention of a viral infection, particularly infection by RSV.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

Suitable therapeutic agents for use in the combination therapies include

(i) RSV nucleocapsid (N)-protein inhibitors;
(ii) other RSV protein inhibitors, such as those that inhibit the phosphoprotein (P) protein and large (L) protein;
(iii) anti-RSV monoclonal antibodies, such as the F-protein antibodies;
(iv) immunomodulating toll-like receptor compounds;
(v) other respiratory virus anti-virals, such as anti-influenza and anti-rhinovirus compounds; and/or
(vi) anti-inflammatory compounds.

The RSV nucleocapsid (N)-protein plays a pivotal role in viral transcription and replication, mediating the interaction between the genomic RNA and the virally encoded RNA-dependent RNA polymerase. The RSV P- and L-proteins are components of RSV's virally encoded RNA-dependent RNA polymerase.

According to a further aspect of the invention, there is provided a compound of the formula (I) or a pharmaceutically acceptable salt thereof as hereinbefore defined in combination with one or more of the therapeutic agents listed as (i) to (vi) above for use in the treatment of RSV.

The Examples that follow serve to illustrate the invention further. The Preparatory Examples relate to the preparation of starting materials and intermediates used to prepare the compounds of the Examples. Neither the Examples nor the Preparatory Examples limit the invention in any way.

### EXAMPLES

Reagents were obtained from commercial sources and were used without further purification. Reactions were performed under anhydrous conditions using anhydrous solvents obtained from commercial sources. All temperatures are in ° C. TLC was performed on aluminium backed silica gel plates with fluorescence indicator at 254 nM (median pore size 60 Å). Microwave reactions were performed using a Biotage Initiator. Flash column chromatography was performed using a Biotage Isolera One system using KP-Sil or Ultra silica gel columns or an Isco CombiFlash Rf using FlashPure, RediSep Rf or RediSep Rf Gold silica gel columns. Reverse phase flash chromatography was performed using an Isco CombiFlash Rf and RP Flash C18 columns. NMR spectra were recorded on a 400, 500, 600 or 700 MHz spectrometer at ambient probe temperature (nominal 298 K). Chemical shifts (6) are given in ppm and calibrated by using the residual peak of the solvent as the internal standard (CDCl₃, δ=7.26 ppm; DMSO-d₆, δ=2.50 ppm). Coupling constants are given in Hertz (Hz). LRMS were recorded using an Advion Plate Express expression^{L} compact mass spectrometer equipped with an APCI ion source.

LCMS analysis was performed using a Waters Acquity UPLC with a Waters X-Select UPLC C18 column (1.7 μm; 2.1×30 mm) and a 3 minute (Method A) or 10 minute method (Method B), or an Agilent UPLC with a Waters X-Select C18 (2.5 μm; 4.6×30 mm) and a 3 minute (Method C) or 10 minute method (Method D). Performed at 40° C. at 0.77 mL/min with a linear 5-95% acetonitrile gradient appropriate for the lipophilicity of the compound. The aqueous portion of the mobile phase was 0.1% formic acid. LC-UV chromatograms were recorded using a Waters Acquity photodiode array detector between 210 and 400 nm. Mass spectra were recorded using a Waters Acquity QDa detector with ESI switching between positive and negative ion mode.

Preparatory examples (3S)-3-amino-5-phenyl-1,3-dihydro-1,4-benzodiazepin-2-one and (3S)-3-amino-9-fluoro-5-phenyl-1,3-dihydro-1,4-benzodiazepin-2-one were prepared using methods described in WO/2004/026843, WO/2005/090319, and WO/2017/015449.

Abbreviations

| | |
|---|---|
| APCI | Atmospheric pressure chemical ionization |
| BINAP | (±)-2,2′-Bis(diphenylphosphino)-1,1′-binaphthalene |
| DIPEA | N,N-Diisopropylethylamine |
| DMF | N,N-Dimethylformamide |
| DMSO | Dimethyl sulfoxide |
| eq. | equivalents |
| ES | Electrospray ionisation |
| h | Hour(s) |
| HATU | 1-[Bis(dimethylamino)methylene]-1H-1,2,3-triazolo[4,5- |
| | b]pyridinium 3-oxid hexafluorophosphate, N- |
| | [(dimethylamino)-1H-1,2,3-triazolo-[4,5-b]pyridin-1- |
| | ylmethylene]-N-methylmethanaminium hexafluorophosphate |
| | N-oxide |
| LCMS | Liquid chromatography-mass spectrometry |
| LDA | Lithium diisopropylamide |
| LRMS | Low resolution mass spectrometry |
| MTBE | Methyl tert-butyl ether |
| MWI | Microwave irradiation |
| Pd-170 | XPhos Pd(crotyl)Cl |
| Pd₂(dba)₃ | Tris(dibenzylideneacetone)dipalladium(0) |
| rt | room temperature |
| RuPhos | 2-Dicyclohexylphosphino-2′,6′-diisopropoxybiphenyl |
| THF | Tetrahydrofuran |
| XPhos | 2-Dicyclohexylphosphino-2′,4′,6′-triisopropylbiphenyl |
| XPhos | Chloro(2-dicyclohexylphosphino-2′,4′,6′-triisopropyl-1,1′- |
| Pd G2 | biphenyl)[2-(2′-amino-1,1′-biphenyl)]palladium(II) |
| XantPhos | 4,5-Bis(diphenylphosphino)-9,9-dimethylxanthene |

### Preparatory Examples

### 1A Ethyl 1-ethylpyrazole-4-carboxylate

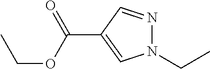

Iodoethane (1.89 mL, 23.56 mmol) was added to a solution of ethyl 1H-pyrazole-4-carboxylate (3.00 g, 21.4 mmol) and K₂CO₃ (3.25 g, 23.6 mmol) in DMF (20 mL) and the reaction mixture stirred at rt for 23 h. The reaction mixture was diluted with water (50 mL), extracted with EtOAc (3×30 mL), the combined organics washed with water and brine (30 mL each), dried (Na₂SO₄), and concentrated under reduced pressure. Purification by flash chromatography (10-50% EtOAc/heptane) afforded a white solid (3.10 g, 86%). ¹H NMR (400 MHz, DMSO-d₆) δ 8.33 (d, J=0.7 Hz, 1H), 7.83 (d, J=0.8 Hz, 1H), 4.25-4.12 (m, 4H), 1.37 (t, J=7.3 Hz, 3H), 1.25 (t, J=7.1 Hz, 3H). LRMS m/z 169.0 [M+H]⁺

### 2A Ethyl 1-ethyl-3-methyl-1H-pyrazole-4-carboxylate

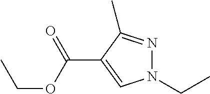

NaH (60% in mineral oil; 0.300 g, 7.50 mmol) was added portionwise to a solution of ethyl 3-methyl-1H-pyrazole-4-carboxylate (1.00 g, 6.49 mmol) in DMF (10 mL) at 0° C. The reaction mixture was stirred for 5 min, iodoethane (0.60 mL, 7.42 mmol) added and the reaction mixture stirred at rt overnight. The reaction mixture was quenched with water (50 mL), diluted with MTBE (50 mL), separated and the aqueous phase extracted with MTBE (2×20 mL). The combined organics were washed with 1:1 water/brine (2×50 mL), dried (MgSO₄), and the solvent removed under reduced pressure. Purification by flash chromatography (0-30% EtOAc/isohexanes) afforded a pale yellow oil (840 mg, 47%). Material was ˜1:0.8 mixture of the desired regioisomer with ethyl 1-ethyl-5-methyl-1H-pyrazole-4-carboxylate as the minor product. Used without further purification. Ratio ascertained by ¹H NMR based upon 6 pyrazole CH signal: ¹H NMR (500 MHz, CDCl₃) δ 7.83 (s, 1H), 4.27 (q, J=7.1 Hz, 2H), 4.11 (q, J=7.3 Hz, 2H), 2.46 (s, 3H), 1.48 (t, J=7.3 Hz, 3H), 1.34 (t, J=7.1 Hz, 3H). LCMS (method A) m/z 183.1 [M+H]⁺ at 1.00 min. Minor product: ethyl 1-ethyl-5-methyl-1H-pyrazole-4-carboxylate. ¹H NMR (500 MHz, CDCl₃) δ 7.84 (s, 1H), 4.28 (q, J=7.1 Hz, 2H), 4.11 (q, J=7.3 Hz, 2H), 2.54 (s, 3H), 1.41 (t, J=7.3 Hz, 3H), 1.34 (t, J=7.1 Hz, 3H). LCMS (method A) m/z 183.1 [M+H]⁺ at 1.00 min.

### 3A Ethyl 1-propan-2-ylpyrazole-4-carboxylate

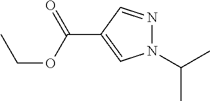

Cs₂CO₃ (3.7 g, 10.7 mmol) and 2-bromopropane (1.62 g, 13.2 mmol) were added to a solution of ethyl 1H-pyrazole-4-carboxylate (1.5 g, 10.7 mmol) in DMF (15 mL) and heated at 60° C. for 2 h. The reaction mixture was diluted with water (150 mL) and extracted with MTBE (2×100 mL). The combined organics were washed with brine (50 mL), dried (Na₂SO₄) and the solvent removed under reduced pressure. The residue was purified by flash chromatography (5-60% EtOAc/isohexanes). Colourless oil (1.65 g, 81%). ¹H NMR (500 MHz, CDCl₃) δ 7.92 (s, 1H), 7.91 (s, 1H), 4.59-4.49 (m, 1H), 4.28 (q, J=7.1 Hz, 2H), 1.52 (d, J=6.7 Hz, 6H), 1.33 (t, J=7.1 Hz, 3H). LCMS (method C): m/z 183.2 [M+H]⁺ at 1.03 min.

### 4A Ethyl 5-amino-1-cyclopropylpyrazole-4-carboxylate

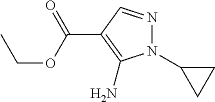

NEt₃ (2.57 mL, 18.42 mmol) was added dropwise over 45 min to a stirred solution of cyclopropylhydrazine hydrochloride (1.00 g, 9.21 mmol) and ethyl (ethoxymethylene)cyanoacetate (1.56 g, 9.21 mmol) in EtOH (10 mL) at rt, then heated at 40° C. for 16 h. The volatiles were removed under reduced pressure, the residue dissolved in CH₂Cl₂ (30 mL), washed with water (2×20 mL) and brine (20 mL), dried (Na₂SO₄) and the solvent removed under reduced pressure. Purification by flash chromatography (30% EtOAc:heptane) afforded a yellow oil (725 mg, 40%). ¹H NMR (400 MHz, DMSO-d₆) δ 7.37 (s, 1H), 6.23 (s, 2H), 4.15 (q, J=7.1 Hz, 2H), 3.26 (tt, J=6.8, 4.1 Hz, 1H), 1.23 (t, J=7.1 Hz, 3H), 1.02-0.86 (m, 4H). LRMS: 196.2 [M+H]⁺

### 5A Ethyl 5-bromo-1-cyclopropylpyrazole-4-carboxylate

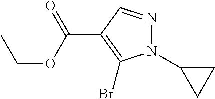

A solution of copper(II) bromide (1001 mg, 4.48 mmol) in MeCN (7.7 mL) was cooled to 0° C. t-Butyl nitrite (0.64 mL, 5.38 mmol) was added followed by dropwise addition of intermediate 4A (700 mg, 3.59 mmol) in MeCN (7.7 mL) over 30 min. The reaction was stirred for 30 min at 0° C., the ice bath removed, then stirred for 16 h at rt. The mixture was poured into a solution of 6 M aq. HCl (20 mL) and extracted with CH₂Cl₂ (3×20 mL). The organics were washed with brine, dried (Na₂SO₄) and the solvent removed under reduced pressure. Purification by flash chromatography (0-10% EtOAc:heptane) afforded a colourless oil (694 mg, 75%). ¹H NMR (400 MHz, DMSO-d₆) δ 7.92 (s, 1H), 4.23 (q, J=7.1 Hz, 2H), 3.75-3.65 (m, 1H), 1.27 (t, J=7.1 Hz, 3H), 1.12-1.03 (m, 4H). LRMS: 259.1/261.1 [M+H]⁺

### 6A Ethyl 5-bromo-1-(oxan-4-yl)pyrazole-4-carboxylate

### 7A Ethyl 3-bromo-1-(oxan-4-yl)pyrazole-4-carboxylate

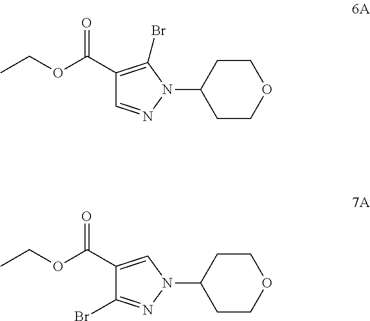 [SMILES: CCOC(=O)c1cnn(C2CCOCC2)c1Br ; CCOC(=O)c1cn(C2CCOCC2)nc1Br]

Oxan-4-yl 4-methylbenzenesulfonate (1.67 g, 6.53 mmol) was added to a solution of ethyl 3-bromo-1H-pyrazole-4-carboxylate (1.30 g, 5.93 mmol) and Cs₂CO₃ (2.63 g, 8.01 mmol) in DMF (10 mL) and heated at 80° C. for 16 h. Upon cooling to rt, water (40 mL) was added and the mixture extracted with EtOAc (3×20 mL). The organics were washed with water and brine (20 mL each), dried (Na₂SO₄) and the solvent removed under reduced pressure. Purification by flash chromatography (10-80% EtOAc:heptane) afforded intermediate 6A as a white solid (445 mg, 25%). ¹H NMR (400 MHz, DMSO-d₆) δ 8.05 (s, 1H), 4.66 (tt, J=11.3, 4.3 Hz, 1H), 4.24 (q, J=7.1 Hz, 2H), 4.01-3.91 (m, 2H), 3.50 (td, J=12.0, 2.1 Hz, 2H), 2.08-1.94 (m, 2H), 1.83 (ddd, J=12.6, 4.4, 2.0 Hz, 2H), 1.27 (t, J=7.1 Hz, 3H). LRMS: 303.0/305.0 [M+H]⁺.

Intermediate 7A (second eluting regioisomer) was obtained as a white solid (1018 mg, 57%). ¹H NMR (400 MHz, DMSO-d₆) δ 8.43 (s, 1H), 4.45 (dd, J=10.5, 5.0 Hz, 1H), 4.22 (q, J=7.1 Hz, 2H), 4.00-3.88 (m, 2H), 3.48-3.36 (m, 2H), 2.04-1.83 (m, 4H), 1.27 (t, J=7.1 Hz, 3H). LRMS: 303.4/305.4 [M+H]⁺.

### 8A Ethyl 3-bromo-1-(4,4-difluorocyclohexyl)pyrazole-4-carboxylate

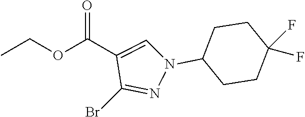 [SMILES: CCOC(=O)c1cn(C2CCC(F)(F)CC2)nc1Br]

Prepared by an analogous procedure to that described for intermediate 6A. ¹H NMR (400 MHz, CDCl₃) δ 7.86 (s, 1H), 4.28 (q, J=7.1 Hz, 2H), 4.25-4.12 (m, 1H), 2.36-2.04 (m, 6H), 2.04-1.73 (m, 2H), 1.32 (t, J=7.1 Hz, 3H). LRMS: 317.3/319.3 [M+H]⁺.

### 9A Ethyl 5-bromo-1-(oxetan-3-yl)pyrazole-4-carboxylate

### 10A Ethyl 3-bromo-1-(oxetan-3-yl)pyrazole-4-carboxylate

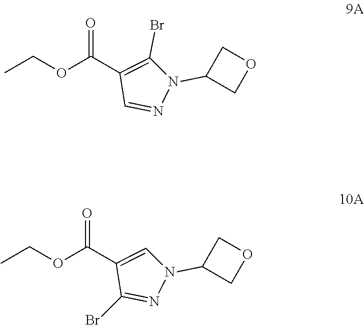

Prepared by an analogous procedure to that described for intermediate 6A with ethyl 3-bromo-1-pyrazole-4-carboxylate (1.70 g, 7.76 mmol), oxetan-3-yl 4-methylbenzene sulfonate (1.95 g, 8.50 mmol) and Cs₂CO₃ (3.43 g, 10.48 mmol) and heating at 90° C. for 22 h. Purification by flash chromatography (10-100% EtOAc:heptane) afforded intermediate 9A (first eluting regioisomer) as a white solid (640 mg, 30%). ¹H NMR (400 MHz, DMSO-d₆) δ 8.15 (s, 1H), 5.75 (tt, J=7.4, 6.2 Hz, 1H), 4.98-4.85 (m, 4H), 4.24 (q, J=7.1 Hz, 2H), 1.28 (t, J=7.1 Hz, 3H). LRMS: 275.5/277.5 [M+H]⁺.

Intermediate 10A: (Second eluting regioisomer), white solid (1.06 g, 50%). ¹H NMR (400 MHz, DMSO-d₆) δ 8.50 (s, 1H), 5.60 (tt, J=7.5, 6.1 Hz, 1H), 4.94-4.77 (m, 4H), 4.23 (q, J=7.1 Hz, 2H), 1.27 (t, J=7.1 Hz, 3H). LRMS: 275.5/277.5 [M+H]⁺.

### 11A Ethyl 3-bromo-1-ethylpyrazole-4-carboxylate

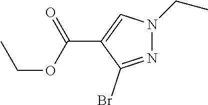

Ethyl iodide (2.02 mL, 25.11 mmol) was added to a solution of ethyl 3-bromo-1H-pyrazole-4-carboxylate (5.00 g, 22.83 mmol) and K₂CO₃ (4.10 g, 29.67 mmol) in DMF (35 mL) and the reaction stirred at rt for 16 h. Water (100 mL) was added, and the mixture extracted with EtOAc (3×50 mL). The organics were washed with water (2×50 mL) and brine (50 mL), dried (Na₂SO₄), and the solvent removed under reduced pressure. Purification by flash chromatography (10-25% EtOAc:heptane) afforded a white solid (3.70 g, 66%). ¹H NMR (400 MHz, DMSO-d₆) δ 8.39 (s, 1H), 4.21 (q, J=7.1 Hz, 2H), 4.15 (q, J=7.3 Hz, 2H), 1.36 (t, J=7.3 Hz, 3H), 1.26 (t, J=7.1 Hz, 3H). LRMS: 247.0/249.0 [M+H]⁺.

### 12A Ethyl 3-bromo-1-(2,2,2-trifluoroethyl)pyrazole-4-carboxylate

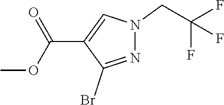

Prepared by an analogous procedure to that described for intermediate 11A using ethyl 3-bromo-1H-pyrazole-4-carboxylate (1.50 g, 6.85 mmol), 1,1,1-trifluoro-2-iodoethane (1.35 mL, 13.7 mmol) and Cs₂CO₃ (4.46 g, 13.7 mmol) in DMF (7 mL) with heating at 70° C. for 3 h, then 40° C. for 16 h. ¹H NMR (400 MHz, CDCl₃) δ 8.01 (s, 1H), 4.71 (q, J=8.1 Hz, 2H), 4.35 (q, J=7.1 Hz, 2H), 1.39 (t, J=7.1 Hz, 3H). LRMS: 301.2/303.2 [M+H]⁺.

### 13A 3-Bromo-1-(4,4-difluorocyclohexyl)pyrazole-4-carboxylic acid

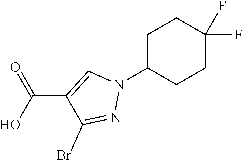 [SMILES: O=C(O)c1cn(C2CCC(F)(F)CC2)nc1Br]

A solution of intermediate 8A (580 mg, 1.72 mmol) and LiOH (1 M aq., 6.88 mL, 6.88 mmol) in THF:MeOH (1:1; 14 mL) was heated at 55° C. for 1 h. The volatiles were removed under reduced pressure, and the residue acidified to pH≈2 with HCl (1 M aq.) then extracted with EtOAc (3×15 mL). The combined organic extracts were washed with water and brine (15 mL each), dried (MgSO₄), and the solvent was removed under reduced pressure to afford a white solid (464 mg, 87%). 1H NMR (400 MHz, DMSO-d₆) δ 12.57 (s, 1H), 8.36 (s, 1H), 4.47-4.38 (m, 1H), 2.23-1.78 (m, 8H). LRMS: 289.1/291.1 [M+H]⁺.

The following intermediate compounds were prepared by the same general procedure described for intermediate 13A.

| Preparatory | | | ¹H NMR δ | LRMS |
|---|---|---|---|---|
| Example | Structure | Name | (400 MHz, DMSO-d₆) | m/z |
| 13B | 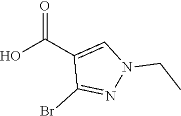 | 3-Bromo-1- ethylpyrazole-4- carboxylic acid | 12.61 (s, 1H), 8.32 (s, 1H), 4.14 (q, J = 7.3 Hz, 2H), 1.36 (t, J = 7.3 Hz, 3H). | 219.1/ 221.1 [M + H]⁺ |
| 13C | 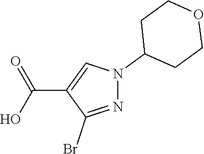 [SMILES: O=C(O)c1cn(C2CCOCC2)nc1Br] | 3-Bromo-1-(oxan-4- yl)pyrazole-4- carboxylic acid | 12.64 (s, 1H), 8.36 (s, 1H), 4.51-4.37 (m, 1H), 3.94 (ddd, J = 11.8, 3.9, 2.0 Hz, 2H), 3.45-3.41 (m, 2H), 2.03-1.85 (m, 4H). | 275.3/ 277.3 [M + H]⁺ |
| 13D | 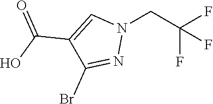 [SMILES: O=C(O)c1cn(CC(F)(F)F)nc1Br] | 3-Bromo-1-(2,2,2- trifluoroethyl) pyrazole-4- carboxylic acid | 12.92 (s, 1H), 8.44 (s, 1H), 5.21 (q, J = 9.0 Hz, 2H) | 273.2/ 275.1 [M + H]⁺ |
| 13E | 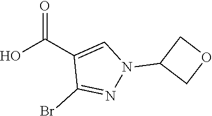 [SMILES: O=C(O)c1cn(C2COC2)nc1Br] | 3-Bromo-1-(oxetan- 3-yl)pyrazole-4- carboxylic acid | δ 12.71 (s, 1H), 8.43 (s, 1H), 5.59 (tt, J = 7.5, 6.1 Hz, 1H), 4.98-4.71 (m, 4H). | 247.3/ 249.2 [M + H]⁺ |

### 14A 3-Bromo-1-tert-butylpyrazole-4-carboxylic acid

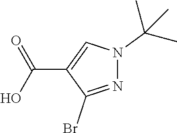

A solution of ethyl 3-bromo-1H-pyrazole-4-carboxylate (1.00 g, 4.57 mmol) in 3-methyl-1-butanol (4 mL) was warmed to 30° C. and then sulfuric acid (0.97 mL, 18.26 mmol) added slowly. The reaction was stirred for 30 min at 30° C., then heated to 80° C. for 1.5 h. The reaction was cooled to rt, diluted with EtOAc (15 mL) and the organic layer separated, washed with water (10 mL), dried (MgSO₄), and the solvent removed under reduced pressure to afford crude ethyl 3-bromo-1-tert-butylpyrazole-4-carboxylate as a colourless oil (1.40 g,) which was used without further purification. A solution of crude ethyl 3-bromo-1-tert-butylpyrazole-4-carboxylate (1.40 g, 5.09 mmol) and LiOH (1 M aq, 10.18 mL, 10.18 mmol) in THF:MeOH (1:1, 20 mL) was heated at 55° C. for 1 h. The volatiles were removed under reduced pressure, and the residue acidified to pH≈2 with 1 M HCl (aq.) then extracted with EtOAc (3×15 mL). The combined organic extracts were washed with water and brine (15 mL each), dried (MgSO₄) and concentrated under reduced pressure to afford a white solid (815 mg, 72%). ¹H NMR (400 MHz, DMSO-d₆) δ 12.59 (s, 1H), 8.31 (s, 1H), 1.51 (s, 9H). LRMS: 247.3/249.3 [M+H]⁺.

### 15A Benzyl 3-bromo-1-(2,2,2-trifluoroethyl)pyrazole-4-carboxylate

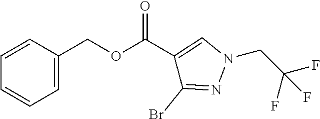 [SMILES: O=C(OCc1ccccc1)c1cn(CC(F)(F)F)nc1Br]

1,8-Diazabicyclo[5.4.0]undec-7-ene (0.15 mL, 0.97 mmol) was added to a solution of intermediate 13D (204 mg, 0.75 mmol) in DMSO (3 mL) under a nitrogen atmosphere and stirred at rt for 5 min. Benzyl bromide (0.09 mL, 0.75 mmol) in DMSO (3 mL) was added, and the reaction stirred at rt for 2 h. The reaction was quenched with water and brine (20 mL each) and extracted with EtOAc (3×20 mL). The organics were washed with brine (2×20 mL), dried (Na₂SO₄) and the solvent removed under reduced pressure. Purification flash chromatography (60-100% EtOAc:heptane) afforded a white solid (218 mg, 80%). ¹H NMR (400 MHz, DMSO-d₆) δ 8.59 (s, 1H), 7.48-7.32 (m, 5H), 5.28 (s, 2H), 5.21 (q, J=8.9 Hz, 2H). LRMS: 363.3/365.3 [M+H]⁺.

The following intermediate compounds were prepared by the same general procedure described for intermediate 15A.

| Preparatory | | | | LRMS |
|---|---|---|---|---|
| Example | Structure | Name | ¹H NMR δ (400 MHz) | m/z |
| 15B | 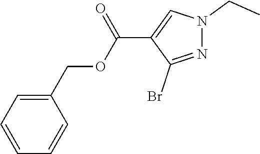 [SMILES: CCn1cc(C(=O)OCc2ccccc2)c(Br)n1] | Benzyl 3-bromo- 1-ethylpyrazole- 4-carboxylate | (DMSO-d₆) δ 8.47 (s, 1H), 7.58-7.24 (m, 5H), 5.27 (s, 2H), 4.16 (q, J = 7.3 Hz, 2H), 1.36 (t, J = 7.3 Hz, 3H). | 309.2/ 311.2 [M + H]⁺ |
| 15C | 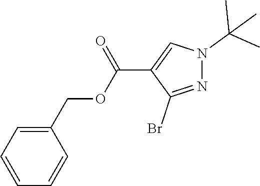 [SMILES: CC(C)(C)n1cc(C(=O)OCc2ccccc2)c(Br)n1] | Benzyl 3-bromo- 1-tert- butylpyrazole-4- carboxylate | (DMSO-d₆) δ 8.41 (s, 1H), 7.52-7.28 (m, 5H), 5.28 (s, 2H), 1.52 (s, 8H). | 337.5/ 339.5 [M + H]⁺ |
| 15D | 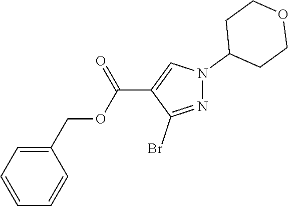 [SMILES: O=C(OCc1ccccc1)c1cn(C2CCOCC2)nc1Br] | Benzyl 3-bromo- 1-(oxan-4- yl)pyrazole-4- carboxylate | (DMSO-d₆) δ 8.50 (s, 1H), 7.53-7.22 (m, 5H), 5.27 (s, 2H), 4.56-4.37 (m, 1H), 3.99-3.88 (m, 2H), 3.50- 3.39 (m, 2H), 2.00-1.77 (m, 5H). | 365.5/ 367.5 [M + H]⁺ |
| 15E | 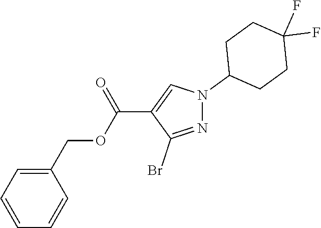 [SMILES: O=C(OCc1ccccc1)c1cn(C2CCC(F)(F)CC2)nc1Br] | Benzyl 3-bromo- 1-(4,4- difluorocyclohexyl) pyrazole-4- carboxylate | (CDCl₃) δ 7.91 (s, 1H), 7.49- 7.30 (m, 5H), 5.30 (s, 2H), 4.21 (t, J = 11.5 Hz, 1H), 2.36-1.76 (m, 8H). | 399.4/ 401.3 [M + H]⁺ |
| 15F | 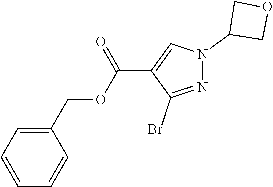 [SMILES: O=C(OCc1ccccc1)c1cn(C2COC2)nc1Br] | Benzyl 3-bromo- 1-(oxetan-3- yl)pyrazole-4- carboxylate | (DMSO-d₆) δ 8.57 (s, 1H), 7.52-7.29 (m, 5H), 5.60 (tt, J = 7.5, 6.2 Hz, 1H), 5.27 (s, 2H), 4.94-4.78 (m, 4H) | 337.5/ 339.5 [M + H]⁺ |

### 16A tert-Butyl (6-chloro-5-fluoropyridin-3-yl)(methyl)carbamate

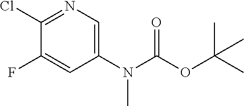 [SMILES: CN(C(=O)OC(C)(C)C)c1cnc(Cl)c(F)c1]

A vial was charged with 5-bromo-2-chloro-3-fluoropyridine (100 ng, 0.475 mmol), tert-butyl methylcarbamate (75 mg, 0.572 mmol) and Cs₂CO₃ (217 mg, 0.665 mmol), 1,4-dioxane (2 mL) added and the reaction mixture was degassed with nitrogen. Pd₂(dba)₃ (9 mg, 9.83 μmol) and Xantphos (22 mg, 0.038 mmol) were added, the reaction mixture was evacuated and purged with nitrogen (×3) and heated at 110° C. overnight. Water (10 mL) and EtOAc (10 mL) were added and the phases separated. The aqueous phase was extracted with EtOAc (2×10 mL) and the combined organic extracts were washed with brine (20 mL), dried (MgSO₄), and the solvent removed under reduced pressure. The residue was purified by flash chromatography (0-20% EtOAc/isohexanes) to afford a pale yellow gum (52 mg, 41%). ¹H NMR (500 MHz, CDCl₃) δ 8.17 (d, J=2.4 Hz, 1H), 7.57 (d, J=9.6 Hz, 1H), 3.31 (s, 3H), 1.50 (s, 91). LCMS (method A): m/z 261.1 [M+H]⁺ at 1.54 min.

### 17A Ethyl 5-(3,5-difluoropyridin-2-yl)-1-ethylpyrazole-4-carboxylate

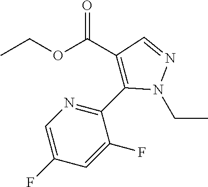 [SMILES: CCOC(=O)c1cnn(CC)c1-c1ncc(F)cc1F]

A solution of intermediate 1A (500 mg, 2.97 mmol) in anhydrous THE (5 mL) was degassed with nitrogen. The solution was cooled to −78° C. and LDA (2.0 M in THF; 1.8 mL, 3.60 mmol) added, stirred at −78° C. for 1 min then zinc (II) chloride (2.0 M in 2-Me THF; 1.8 mL, 3.60 mmol) added. The reaction was warmed to rt and degassed with nitrogen. 2-Bromo-3,5-difluoropyridine (690 mg, 3.56 mmol) and Pd(PPh₃)₄ (172 mg, 0.15 mmol) were added, the reaction mixture evacuated and purged with nitrogen, then heated at 70° C. under nitrogen overnight. 1 M aq. HCl (50 mL) and EtOAc (50 mL) were added and the phases separated. The aqueous phase was extracted with EtOAc (2×20 mL), the combined organics washed with brine (100 mL), dried (MgSO₄), and the solvent removed under reduced pressure. The residue was purified by flash chromatography (0-40% MTBE/isohexanes) to afford a pale yellow oil (523 mg, 62%). ¹H NMR (500 MHz, CDCl₃) δ 8.49 (d, J=2.4 Hz, 1H), 8.02 (s, 1H), 7.36 (td, J=8.3, 2.4 Hz, 1H), 4.18 (q, J=7.1 Hz, 2H), 4.10 (q, J=7.3 Hz, 2H), 1.39 (t, J=7.2 Hz, 3H), 1.20 (t, J=7.1 Hz, 3H). LCMS (method A): m/z 282.1 [M+H]⁺ at 1.23 min.

The following intermediate compounds were prepared by the same general procedure described for intermediate 17A.

| Preparatory | | | ¹H NMR δ | LCMS m/z |
|---|---|---|---|---|
| Example | Structure | Name | (500 MHz, CDCl₃) | Method A |
| 17B | 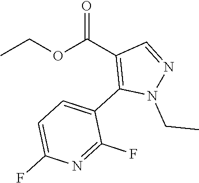 [SMILES: CCOC(=O)c1cnn(CC)c1-c1ccc(F)nc1F] | Ethyl 5-(2,6- difluoropyridin-3- yl)-1- ethylpyrazole-4- carboxylate | 8.06 (s, 1H), 7.97-7.89 (m, 1H), 7.03-6.97 (m, 1H), 4.18 (q, J = 7.1 Hz, 2H), 4.04-3.95 (m, 2H), 1.41 (t, J = 7.3 Hz, 3H), 1.21 (t, J = 7.1 Hz, 3H). | 282.1 [M + H]⁺ at 1.30 min |
| 17C | 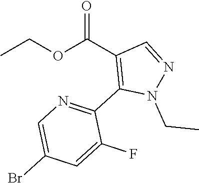 [SMILES: CCOC(=O)c1cnn(CC)c1-c1ncc(Br)cc1F] | Ethyl 5-(4-bromo- 2-fluorophenyl)-1- ethyl-1H- pyrazole-4- carboxylate | 8.65 (dd, J = 1.9, 0.9 Hz, 1H), 8.02 (s, 1H), 7.76 (dd, J = 8.1, 1.9 Hz, 1H), 4.20 (q, J = 7.1 Hz, 2H), 4.13 (q, J = 7.2 Hz, 2H), 1.40 (t, J = 7.2 Hz, 3H), 1.22 (t, J = 7.1 Hz, 3H). | 342.0/ 344.0 [M + H]⁺ at 1.42 min. |
| 17D | 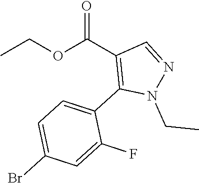 [SMILES: CCOC(=O)c1cnn(CC)c1-c1ccc(Br)cc1F] | Ethyl 5-(4-bromo- 2-fluorophenyl)-1- ethylpyrazole-4- carboxylate | — | 341.0/ 343.0 [M + H]⁺ at 1.56 min. |
| 17E | 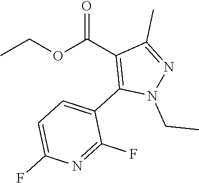 [SMILES: CCOC(=O)c1c(C)nn(CC)c1-c1ccc(F)nc1F] | Ethyl 5-(2,6- difluoropyridin-3- yl)-1-ethyl-3- methyl-1H- pyrazole-4- carboxylate | — | 296.2 [M + H]⁺ at 1.36 min. |
| 17F | 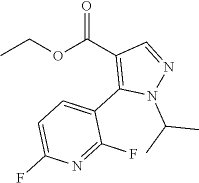 [SMILES: CCOC(=O)c1cnn(C(C)C)c1-c1ccc(F)nc1F] | Ethyl 5-(2,6- difluoropyridin-3- yl)-1-propan-2- ylpyrazole-4- carboxylate | 8.06 (s, 1H), 7.94-7.85 (m, 1H), 6.99 (dd, J = 8.1, 2.8 Hz, 1H), 4.16 (q, J = 7.1 Hz, 2H), 1.51 (d, J = 6.8 Hz, 3H), 1.42 (d, J = 6.6 Hz, 3H), 1.20 (t, J = 7.1 Hz, 3H). | 296.1 [M + H]⁺ at 2.25 min. |
| 17G | 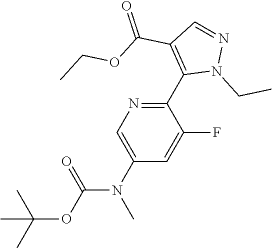 [SMILES: CCOC(=O)c1cnn(CC)c1-c1ncc(N(C)C(=O)OC(C)(C)C)cc1F] | Ethyl 5-(5-((tert- butoxycarbonyl) (methyl)amino)-3- fluoropyridin-2- yl)-1-ethyl-1H- pyrazole-4- carboxylate | 8.60-8.55 (m, 1H), 8.03 (s, 1H), 7.66 (d, J = 10.0 Hz, 1H), 4.19 (q, J = 7.1 Hz, 2H), 4.14 (q, J = 7.2 Hz, 2H), 3.39 (s, 3H), 1.54 (s, 9H), 1.40 (t, J = 7.3 Hz, 3H), 1.21 (t, J = 7.1 Hz, 3H). | 393.6 [M + H]⁺ at 1.56 min. |

### 18A Ethyl 1-cyclopropyl-5-(2,6-difluoropyridin-3-yl)pyrazole-4-carboxylate

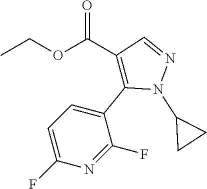 [SMILES: CCOC(=O)c1cnn(C2CC2)c1-c1ccc(F)nc1F]

A solution of intermediate 5A (500 mg, 1.93 mmol), 2,6-difluoropyridine-3-boronic acid (337 mg, 2.12 mmol), K₂CO₃ (2 M aqueous; 4.82 mL, 9.65 mmol) in 1,4-dioxane (7 mL) in a microwave vial was purged with nitrogen for 15 min. Pd(PPh₃)₄ (223 mg, 0.19 mmol) was added, and the sealed vial heated at 110° C. for 17 h. The reaction was cooled to rt, purged with nitrogen for 15 min, additional 2,6-difluoropyridine-3-boronic acid (61.3 mg, 0.39 mmol) and Pd(PPh₃)₄ (55.8 mg, 0.05 mmol) added and heated at 110° C. for a further 4 h. The reaction was cooled to rt, water (20 mL) added, and extracted with EtOAc (3×20 mL). The combined organics were washed with brine (20 mL), dried (Na₂SO₄) and the solvent was removed under reduced pressure. Purification by flash chromatography (10-35% EtOAc:heptane) afforded a yellow oil (206 mg, 18%) as a ˜1:1 ratio by ¹H NMR of product to dehalogenated starting material. Taken forward without further purification. Intermediate 18A: ¹H NMR (400 MHz, DMSO-d₆) δ 8.51-8.42 (m, 1H), 7.97 (s, 1H), 7.42 (ddd, J=8.2, 2.5, 0.7 Hz, 1H), 4.08 (q, J=7.1 Hz, 2H), 3.58 (tt, J=7.4, 3.8 Hz, 1H), 1.13-1.04 (m, 5H), 1.04-0.92 (m, 4H), 0.92-0.83 (m, 2H). LRMS m/z: 294.1 [M+H]⁺. Side product: ethyl 1-cyclopropyl-1H-pyrazole-4-carboxylate: ¹H NMR (400 MHz, DMSO-d₆)¹H NMR (400 MHz, DMSO-d₆) δ 8.37 (s, 1H), 7.81 (d, J=0.7 Hz, 1H), 4.20 (q, J=7.1 Hz, 2H), 3.80 (tt, J=7.5, 3.9 Hz, 1H), 1.25 (t, J=7.1 Hz, 3H), 1.13-0.82 (m, 4H). LRMS m/z: 181.1 [M+H]⁺.

### 19A Benzyl 3-(2,6-difluoropyridin-3-yl)-1-(oxetan-3-yl)pyrazole-4-carboxylate

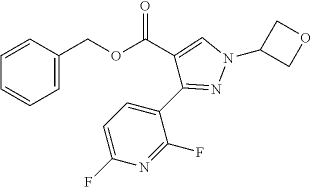 [SMILES: O=C(OCc1ccccc1)c1cn(C2COC2)nc1-c1ccc(F)nc1F]

A microwave vial charged with 2,6-difluoropyridine-3-boronic acid (413 mg, 2.60 mmol), intermediate 15F (585 mg, 1.74 mmol) and XPhos Pd G2 (7.5 mol %; 102 mg, 0.130 mmol) was evacuated and filled with N₂ (3×). THE (10 mL) and K₃PO₄ (2 M aq; 1.74 mL, 3.47 mmol), both degassed with N₂ for ˜15 min, were added, and the vial heated by MWI at 80° C. for 30 min. The volatiles were removed under reduced pressure and the residue purified by flash chromatography (20 to 45% EtOAc:heptane) to afford a yellow oil (520 mg, 81%)¹H NMR (400 MHz, DMSO-d₆) δ 8.68 (s, 1H), 8.28 (q, J=8.5 Hz, 1H), 7.39-7.21 (m, 6H), 5.73-5.61 (m, 1H), 5.18 (s, 2H), 4.96-4.90 (m, 4H). LRMS m/z: [M+H]⁺372.5.

The following intermediate compounds were prepared using an analogous procedure.

| | | | | | Catalyst |
|---|---|---|---|---|---|
| | | | | | loading/ |
| | | | | | Reaction |
| Preparatory | | | | LRMS | time/ |
| Example | Structure | Name | ¹H NMR δ (400 MHz) | m/z | Temperature |
| 19B | 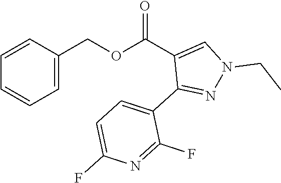 | Benzyl 3-(2,6- difluoropyridin- 3-yl)-1- ethylpyrazole- 4-carboxylate | (DMSO-d₆) δ 8.57 (s, 1H), 8.22 (dt, J = 9.3, 8.0 Hz, 1H), 7.39-7.26 (m, 5H), 7.22 (ddd, J = 8.1, 2.5, 0.6 Hz, 1H), 5.17 (s, 2H), 4.24 (q, J = 7.2 Hz, 2H), 1.43 (t, J = 7.3 Hz, 3H). | 341.1 [M + H]⁺ | 5 mol %/ 2 × 15 min/ 70° C. |
| 19C | 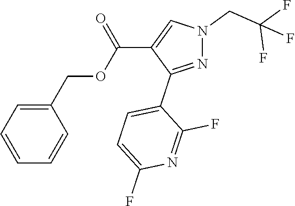 [SMILES: O=C(OCc1ccccc1)c1cn(CC(F)(F)F)nc1-c1ccc(F)nc1F] | Benzyl 3-(2,6- difluoropyridin- 3-yl)-1-(2,2,2- trifluoroethyl) pyrazole-4- carboxylate | (CDCl₃) δ 8.15 (s, 1H), 8.00 (dt, J = 8.9, 7.8 Hz, 1H), 7.34 (dd, J = 5.1, 2.0 Hz, 3H), 7.28 (d, J = 3.4 Hz, 2H), 6.84 (dd, J = 8.1, 2.8 Hz, 1H), 5.20 (s, 2H), 4.76 (q, J = 8.2 Hz, 2H). | 398.4 [M + H]⁺ | 5 mol %/ 30 min/ 80° C. |
| 19D | 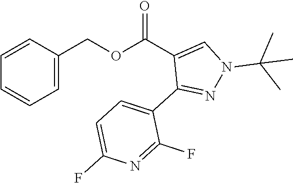 [SMILES: CC(C)(C)n1cc(C(=O)OCc2ccccc2)c(-c2ccc(F)nc2F)n1] | Benzyl 1-tert- butyl-3-(2,6- difluoropyridin- 3-yl)pyrazole- 4-carboxylate | (DMSO-d₆) δ 8.52 (s, 1H), 8.20 (dt, J = 9.4, 8.0 Hz, 1H), 7.40-7.24 (m, 5H), 7.24- 7.16 (m, 1H), 5.16 (s, 2H), 1.58 (s, 9H). | 372.4 [M + H]⁺ | 5 mol %/ 20 min/ 80° C. |
| 19E | 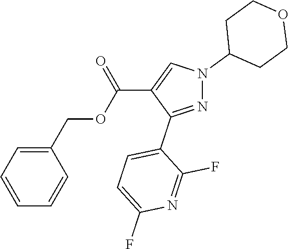 [SMILES: O=C(OCc1ccccc1)c1cn(C2CCOCC2)nc1-c1ccc(F)nc1F] | Benzyl 3-(2,6- difluoropyridin- 3-yl)-1-(oxan- 4-yl)pyrazole- 4-carboxylate | (DMSO-d₆) δ 8.60 (s, 1H), 8.21 (dt, J = 9.3, 8.0 Hz, 1H), 7.42-7.16 (m, 6H), 5.17 (s, 2H), 4.61-4.45 (m, 1H), 4.05-3.91 (m, 2H), 3.51- 3.37 (m, 2H), 2.01 (td, J = 10.7, 9.4, 3.9 Hz, 4H). | 400.3 [M + H]⁺ | 7.5 mol %/ 30 min/ 80° C. |
| 19F | 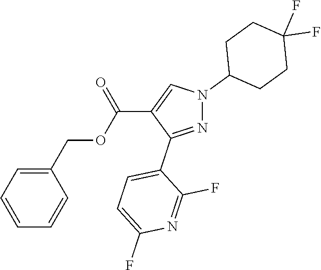 [SMILES: O=C(OCc1ccccc1)c1cn(C2CCC(F)(F)CC2)nc1-c1ccc(F)nc1F] | Benzyl 1-(4,4- difluorocyclohexyl)- 3-(2,6- difluoropyridin- 3-yl)pyrazole- 4-carboxylate | (DMSO-d₆) δ 8.61 (s, 1H), 8.31-8.16 (m, 1H),7.38- 7.17 (m, 6H), 5.16 (s, 2H), 4.53 (s, 1H), 2.24-1.84 (m, 8H). | 434.6 [M + H]⁺ | 5 mol %/ 30 min/ 80° C. |
| 19G | 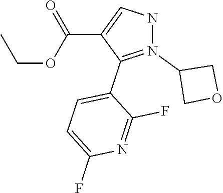 | Ethyl 5-(2,6- difluoropyridin- 3-yl)-1- (oxetan-3- yl)pyrazole-4- carboxylate | (CDCl₃) δ 8.18 (s, 1H), 7.96 (ddd, J = 9.0, 8.2, 7.5 Hz, 1H), 7.01 (ddd, J = 8.2, 2.9, 0.7 Hz, 1H), 5.20-4.96 (m, 4H), 4.86 (t, J = 6.5 Hz, 1H), 4.20 (qd, J = 7.1, 0.9 Hz, 2H), 1.24 (t, J = 7.1 Hz, 3H) | 310.8 [M + H]⁺ | 7.5 mol %/ 30 min/ 80° C. |
| 19H | 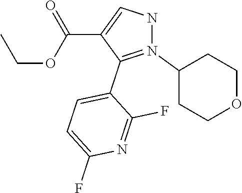 | Ethyl 5-(2,6- difluoropyridin- 3-yl)-1-(oxan- 4-yl)pyrazole- 4-carboxylate | (DMSO-d₆) δ 8.43-8.28 (m, 1H), 8.10 (d, J = 0.5 Hz, 1H), 7.46-7.35 (m, 1H), 4.25- 4.18 (m, 1H), 4.12-3.79 (m, 5H), 2.14-1.81 (m, 4H), 1.67 (d, J = 12.7 Hz, 1H), 1.07 (t, J = 7.1 Hz, 3H). | 338.7 [M + H]⁺ | 7.5 mol %/ 30 min/ 80° C. |

### 20A Ethyl 1-ethyl-5-(2-fluoro-6-(methylamino)pyridin-3-yl)-1H-pyrazole-4-carboxylate

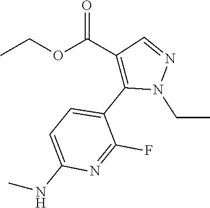 [SMILES: CCOC(=O)c1cnn(CC)c1-c1ccc(NC)nc1F]

NEt₃ (40 μL, 0.287 mmol) and methylamine (2.0 M in THF; 72 μL, 0.144 mmol) were added to a solution of intermediate 17B (40.5 mg, 0.144 mmol) in THE (1 mL) and stirred at rt overnight. Further methylamine (2.0 M in THF; 72 μL, 0.144 mmol) was added and the reaction was stirred at rt overnight. Water (10 mL) and EtOAc (10 mL) were added, the phases were separated, and the aqueous phase extracted with EtOAc (2×5 mL). The combined organics were washed with brine (10 mL), dried (MgSO₄), and the solvent removed under reduced pressure. Purification by flash chromatography (0-50% EtOAc/isohexanes) afforded a colourless gum (18 mg, 42%). ¹H NMR (500 MHz, CDCl₃) δ 8.01 (s, 1H), 7.50 (dd, J=9.5, 8.3 Hz, 1H), 6.34 (dd, J=8.3, 1.9 Hz, 1H), 4.85-4.81 (m, 1H), 4.19 (q, J=7.1 Hz, 2H), 4.04-4.00 (m, 2H), 2.99 (d, J=5.1 Hz, 3H), 1.40 (t, J=7.2 Hz, 3H), 1.23 (t, J=7.1 Hz, 3H). LCMS (method A): m/z 293.2 [M+H]⁺ at 1.19 min.

### 21A Ethyl 1-ethyl-5-(6-(ethylamino)-2-fluoropyridin-3-yl)-1H-pyrazole-4-carboxylate

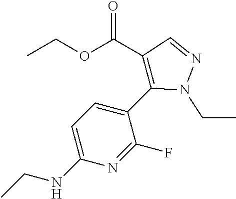 [SMILES: CCNc1ccc(-c2c(C(=O)OCC)cnn2CC)c(F)n1]

DIPEA (130 μl, 0.744 mmol) and ethylamine, (68% in water, 38 μL, 0.466 mmol) were added to a solution of intermediate 17B (100 mg, 0.356 mmol) in THE (1 mL) and the reaction heated at 60° C. overnight. The reaction mixture was diluted with CH₂Cl₂ (10 mL) and water (10 mL), passed through a phase separator and dried under reduced pressure. The crude was purified by flash chromatography (0-50% EtOAc/isohexanes) to afford a colourless gum (57 mg, 48%). ¹H NMR (500 MHz, CDCl₃) δ 8.03 (s, 1H), 7.52 (dd, J=9.3, 8.3 Hz, 1H), 6.36 (dd, J=8.4, 1.7 Hz, 1H), 5.31 (s, 1H), 4.20 (q, J=7.1 Hz, 2H), 4.11-3.98 (m, 2H), 3.38 (q, J=7.2 Hz, 2H), 1.46-1.37 (m, 3H), 1.31 (t, J=7.2 Hz, 3H), 1.24 (t, J=7.1 Hz, 3H). LCMS (method A): m/z 307.1 [M+H]⁺ at 1.31 min.

The following intermediate compounds were prepared using an analogous procedure to that used for intermediate 21A.

| Preparatory | | | ¹H NMR δ | LCMS m/z | Amine eq./ |
|---|---|---|---|---|---|
| Example | Structure | Name | (500 MHz, CDCl₃) | method A | Temp (° C.) |
| 21B | 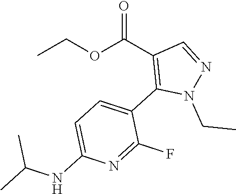 [SMILES: CCOC(=O)c1cnn(CC)c1-c1ccc(NC(C)C)nc1F] | Ethyl 1-ethyl-5- (2-fluoro-6- (isopropylamino) pyridin-3-yl)- 1H-pyrazole-4- carboxylate | 8.02 (s, 1H), 7.50 (dd, J = 10.1, 8.3 Hz, 1H), 6.35-6.28 (m, 1H), 5.31 (s, 1H), 4.20 (q, J = 7.2 Hz, 2H), 4.06-4.02 (m, 2H), 4.00-3.91 (m, 1H), 1.45- 1.38 (m, 3H), 1.33-1.27 (m, 6H), 1.24 (t, J = 7.1 Hz, 3H). | 321.2 [M + H]⁺ at 1.44 min. | 9.8 eq. 50° C. |
| 21C | 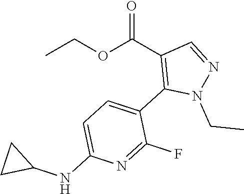 [SMILES: CCOC(=O)c1cnn(CC)c1-c1ccc(NC2CC2)nc1F] | Ethyl 5-(6- (cyclopropyl- amino)-2- fluoropyridin-3- yl)-1-ethyl-1H- pyrazole-4- carboxylate | δ 8.05-8.01 (m, 1H), 7.60 (t, J = 8.8 Hz, 1H), 6.75-6.70 (m, 1H), 4.20 (q, J = 7.1 Hz, 2H), 4.07-4.01 (m, 2H), 2.63- 2.56 (m, 1H), 1.46-1.37 (m, 3H), 1.24 (t, J = 7.1 Hz, 3H), 0.96-0.86 (m, 2H), 0.67- 0.60 (m, 2H). | 319.2 [M + H]⁺ at 1.34 min. | 10 eq. 60° C. |
| 21D | 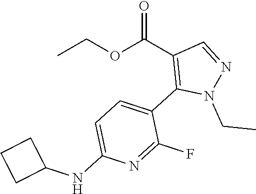 [SMILES: CCOC(=O)c1cnn(CC)c1-c1ccc(NC2CCC2)nc1F] | Ethyl 5-(6- (cyclobutyl- amino)-2- fluoropyridin-3- yl)-1-ethyl-1H- pyrazole-4- carboxylate | — | 333.5 [M + H]⁺ at 1.48 min. | 3 eq. 50° C. |

### 22A Ethyl 1-cyclopropyl-5-[6-(ethylamino)-2-fluoropyridin-3-yl]pyrazole-4-carboxylate

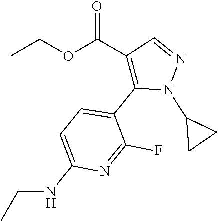 [SMILES: CCNc1ccc(-c2c(C(=O)OCC)cnn2C2CC2)c(F)n1]

A solution of crude intermediate 18A (50 mg, 0.170 mmol), ethylamine (2 M in MeOH; 34 μL, 0.511 mmol) and NEt₃ (95 μL, 0.682 mmol) in THE (0.49 mL) was heated in a sealed vial under nitrogen 50° C. for 18.5 h. Additional ethylamine (2 M in MeOH; 34 μL, 0.511 mmol) was added and the reaction heated for 4 h at 50° C. The volatiles were removed under reduced pressure, and the residue purified by flash chromatography (10-30% EtOAc/heptane) to afford a colourless oil (32.0 mg, 59%). TLC R_{f}0.56 (heptane/EtOAc 1:1). LRMS m/z: 294.1 [M+H]⁺.

### 23A Ethyl 1-cyclopropyl-5-[6-(cyclopropylamino)-2-fluoropyridin-3-yl]pyrazole-4-carboxylate

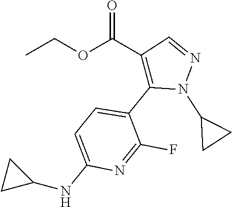 [SMILES: CCOC(=O)c1cnn(C2CC2)c1-c1ccc(NC2CC2)nc1F]

Prepared by an analogous procedure to that described for intermediate 22A using cyclopropylamine (71 μL, 1.02 mmol) and heating at 60° C. for 19 h. TLC R_{f} 0.45 (heptane/EtOAc 1:1). LRMS m/z: 331.3 [M+H]⁺.

### 24A Ethyl 5-[5-(cyclopropylamino)-3-fluoropyridin-2-yl]-1-ethylpyrazole-4-carboxylate

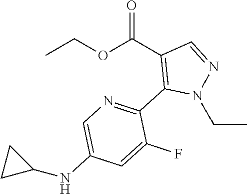 [SMILES: CCOC(=O)c1cnn(C(C)C)c1-c1ncc(NC2CC2)cc1F]

Cyclopropylamine (500 μL, 7.21 mmol) was added to a solution of intermediate 17A (200 mg, 0.711 mmol) and DIPEA (150 μL, 0.861 mmol) in DMSO (1 mL) and heated by MWI at 140° C. for 3 h. Water (20 mL) and EtOAc (20 mL) were added, the phases separated and the aqueous phase extracted with EtOAc (2×10 mL). The combined organics were washed with water and brine (20 mL each), dried (MgSO₄), and the solvent evaporated under reduced pressure. Purification by flash chromatography (10-60% EtOAc/isohexanes) afforded a colourless oil (215 mg, 94%). ¹H NMR (500 MHz, DMSO-d₆) δ 8.04 (s, 1H), 8.00 (s, 1H), 6.99-6.92 (m, 1H), 4.62 (s, 1H), 4.19 (q, J=7.1 Hz, 2H), 4.13 (q, J=7.2 Hz, 2H), 2.51 (s, 1H), 1.64 (s, 1H), 1.39 (t, J=7.1 Hz, 3H), 1.22 (t, J=7.2 Hz, 3H), 0.90-0.82 (m, 2H), 0.65-0.59 (m, 2H). LCMS (method A) m/z 319.2 [M+H]⁺ at 1.30 min.

The following intermediate compounds were prepared by an analogous procedure to that used for intermediate 24A.

| | | | | | Amine eq./ |
|---|---|---|---|---|---|
| Preparatory | | | ¹H NMR δ | LCMS m/z | Reaction |
| Example | Structure | Name | (500 MHz, CDCl₃) | Method A | time |
| 24B | 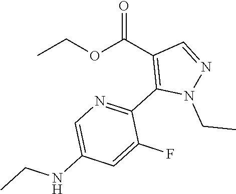 [SMILES: CCNc1cnc(-c2c(C(=O)OCC)cnn2CC)c(F)c1] | Ethyl 1-ethyl- 5-[5- (ethylamino)- 3- fluoropyridin- 2-yl]pyrazole- 4-carboxylate | 7.98 (s, 1H), 7.95-7.91 (m, 1H), 6.65 (dd, J = 11.4, 2.4 Hz, 1H), 4.18 (q, J = 7.1 Hz, 2H), 4.11 (q, J = 7.3 Hz, 2H), 4.09-4.06 (m, 1H), 3.26- 3.17 (m, 2H), 1.37 (t, J = 7.2 Hz, 3H), 1.32 (t, J = 7.1 Hz, 3H), 1.21 (t, J = 7.1 Hz, 3H). | 307.1 [M + H]⁺ at 1.24 min. | 10 eq., 1 h |
| 24C | 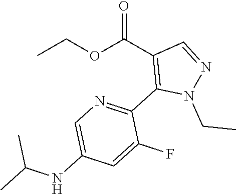 [SMILES: CCOC(=O)c1cnn(CC)c1-c1ncc(NC(C)C)cc1F] | Ethyl 1-ethyl- 5-[3-fluoro-5- (propan-2- ylamino) pyridin-2- yl]pyrazole-4- carboxylate | 8.00 (s, 1H), 7.94-7.90 (m, 1H), 6.65 (d, J = 11.4 Hz, 1H), 4.19 (q, J = 7.1 Hz, 2H), 4.13 (q, J = 7.2 Hz, 2H), 3.65 (p, J = 6.3 Hz, 1H), 1.59- 1.54 (m, 1H), 1.39 (t, J = 7.2 Hz, 3H), 1.32-1.27 (m, 6H), 1.22 (t, J = 7.1 Hz, 3H). | 321.2 [M + H]⁺ at 1.34 min. | 6.5 eq., 3 h |

### 25A Ethyl 1-ethyl-5-[2-fluoro-6-[(1-methylcyclopropyl)amino]pyridin-3-yl]pyrazole-4-carboxylate

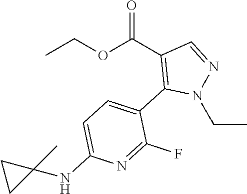 [SMILES: CCOC(=O)c1cnn(C(C)C)c1-c1ccc(NC2(C)CC2)nc1F]

Prepared by an analogous procedure to that described for intermediate 24A with intermediate 17B (100 mg, 0.338 mmol), 1-methylcyclopropan-1-amine hydrochloride (182 mg, 1.69 mmol) and DIPEA (350 μL, 2.01 mmol) and heating by MWI at 120° C. for 1 h. Following an analogous workup, purification by flash chromatography on RP Flash C18 (25-75% MeCN/Water 0.1% formic acid) afforded a pale yellow gum (70 mg, 62%). ¹H NMR (500 MHz, DMSO-d₆) δ 8.02 (s, 1H), 7.55 (dd, J=9.5, 8.2 Hz, 1H), 6.65 (dd, J=8.2, 1.9 Hz, 1H), 4.19 (q, J=7.1 Hz, 2H), 4.08-3.96 (m, 2H), 1.44 (s, 3H), 1.41 (t, J=7.2 Hz, 3H), 1.23 (t, J=7.1 Hz, 3H), 0.88-0.85 (m, 2H), 0.78-0.75 (m, 2H). LCMS (method A): m/z 333.2 [M+H]⁺ at 1.44 min.

### 26A Ethyl 5-[6-(cyclopropylamino)-2-fluoropyridin-3-yl]-1-propan-2-ylpyrazole-4-carboxylate

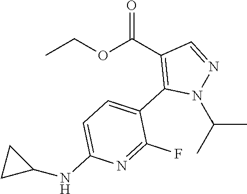 [SMILES: CCOC(=O)c1cnn(C(C)(C)C)c1-c1ccc(NC2CC2)nc1F]

A solution of intermediate 17F (100 mg, 0.339 mmol) and cyclopropylamine (117 μL, 1.693 mmol) in DMSO (1 mL) was heated at 50° C. for 16 h. Analogous workup and purification to that described for intermediate 24A afforded a colourless oil (100 mg, 82%). ¹H NMR (500 MHz, CDCl₃) δ 8.02 (s, 1H), 7.57-7.50 (m, 1H), 6.68 (dd, J=8.2, 1.8 Hz, 1H), 5.33 (s, 1H), 4.33-4.24 (m, 1H), 4.17 (q, J=7.1 Hz, 2H), 2.61-2.53 (m, 1H), 1.49 (d, J=6.6 Hz, 3H), 1.39 (d, J=6.6 Hz, 3H), 1.21 (t, J=7.1 Hz, 3H), 0.87-0.80 (m, 2H), 0.64-0.59 (m, 2H). LCMS (method A) m/z 333 [M+H]⁺ at 1.35 min.

### 27A Ethyl 1-ethyl-5-[2-fluoro-4-(propan-2-ylamino)phenyl]pyrazole-4-carboxylate

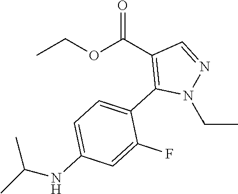 [SMILES: CCOC(=O)c1cnn(C(C)C)c1-c1ccc(NC(C)C)cc1F]

A microwave vial charged with intermediate 17D (100 mg, 0.29 mmol), Pd₂(dba)₃ (13 mg, 0.010 mmol), BINAP (18 mg, 0.030 mmol), potassium tert-butoxide (43 mg, 0.38 mmol) and isopropylamine (29 μL, 0.35 mmol) was evacuated and purged with N₂, then DMSO (1 mL) added. The reaction mixture was degassed with nitrogen, then heated at 110° C. for 1 h. Water (20 mL) and EtOAc (10 mL) were added, the phases separated, and the aqueous phase extracted with EtOAc (2×10 mL). The combined organic extracts were washed with water, brine (20 mL each), dried (MgSO₄), and the solvent removed under reduced pressure. The residue was purified by flash chromatography (0-50% EtOAc/isohexanes) to afford a colourless gum (22.0 mg, 23%). LCMS (method A) m/z 320.2 [M+H]⁺ at 1.52 min.

### 28A Ethyl 1-ethyl-5-(6-(ethylamino)-2-fluoropyridin-3-yl)-3-methyl-1H-pyrazole-4-carboxylate

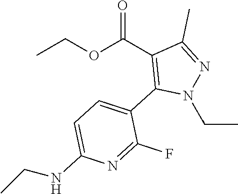 [SMILES: CCNc1ccc(-c2c(C(=O)OCC)c(C)nn2C(C)C)c(F)n1]

Prepared by an analogous procedure to intermediate 21A using intermediate 17E (100 mg, 0.254 mmol), DIPEA (0.1 mL, 0.573 mmol) and ethylamine, 68% in water (0.4 mL, 4.9 mmol) in THE (1 mL). The reaction was performed at 50° C. for 2 h. ¹H NMR (500 MHz, CDCl₃) δ 7.49-7.39 (m, 1H), 6.34-6.28 (m, 1H), 4.15 (q, J=7.1 Hz, 2H), 3.99 (q, J=7.3 Hz, 2H), 3.42-3.33 (m, 2H), 2.53 (s, 3H), 1.38 (t, J=7.2 Hz, 3H), 1.29 (t, J=7.2 Hz, 3H), 1.17 (t, J=7.1 Hz, 3H). LCMS (method A) m/z 321.2 [M+H]⁺ at 1.36 min.

### 29A Ethyl 5-(5-((tert-butoxycarbonyl)(ethyl)amino)-3-fluoropyridin-2-yl)-1-ethyl-1H-pyrazole-4-carboxylate

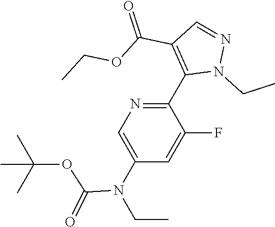 [SMILES: CCOC(=O)c1cnn(C(C)C)c1-c1ncc(N(CC)C(=O)OC(C)(C)C)cc1F]

A vial was charged with intermediate 17C (130 mg, 0.380 mmol), tert-butyl ethylcarbamate (56 mg, 0.386 mmol) and CsCO₃ (173 mg, 0.532 mmol). 1,4-Dioxane (2 mL) was added and the reaction mixture was degassed with nitrogen. Pd₂(dba)₃ (7 mg, 7.64 μmol) and Xantphos (11 mg, 0.019 mmol) were added, the reaction mixture evacuated and purged with nitrogen (3×), then heated at 110° C. overnight. Water (20 mL), brine (10 mL) and EtOAc (20 mL) were added, separated, and the aqueous phase extracted with EtOAc (2×10 mL). The combined organic extracts were washed with brine (20 mL), dried (MgSO₄), and the solvent was removed under reduced pressure. The residue was purified by flash chromatography (0-50% EtOAc/isohexanes) to afford a pale-yellow gum (144 mg, 88%). ¹H NMR (500 MHz, CDCl₃) δ 8.55-8.50 (m, 1H), 8.03 (s, 1H), 7.59 (d, J=10.3 Hz, 1H), 4.19 (q, J=7.1 Hz, 2H), 4.14 (q, J=7.2 Hz, 2H), 3.81 (q, J=7.1 Hz, 2H), 1.52 (s, 9H), 1.39 (t, J=7.3 Hz, 3H), 1.29 (t, J=7.1 Hz, 3H), 1.20 (t, J=7.1 Hz, 3H). LCMS (method A): m/z 407.2 [M+H]⁺ at 1.63 min.

### 30A Benzyl 3-[6-(cyclopropylamino)-2-fluoropyridin-3-yl]-1-ethylpyrazole-4-carboxylate

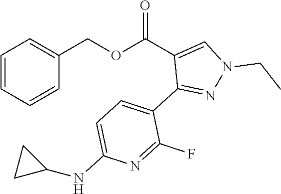 [SMILES: CCn1cc(C(=O)OCc2ccccc2)c(-c2ccc(NC3CC3)nc2F)n1]

Cyclopropylamine (0.2 mL, 2.91 mmol) and DIPEA (0.2 mL, 1.17 mmol) were added to a solution of intermediate 19B (200 mg, 0.58 mmol) in DMSO (3 mL) and the reaction heated by MWI at 120° C. for 30 min. Water (40 mL) and brine (40 mL) were added and the mixture extracted with EtOAc (3×40 mL). The combined organic extracts were washed with brine (2×40 mL), dried (MgSO₄), and the solvent removed under reduced pressure. Purification by flash chromatography (1:9 to 3:7 EtOAc:heptane) afforded benzyl 3-[2-(cyclopropylamino)-6-fluoropyridin-3-yl]-1-ethylpyrazole-4-carboxylate as the first eluting regioisomer (colourless oil, 90 mg, 41%) followed by intermediate 30A as the second eluting regioisomer (white oil, 95 mg, 43%). Benzyl 3-[2-(cyclopropylamino)-6-fluoropyridin-3-yl]-1-ethylpyrazole-4-carboxylate: ¹H NMR (400 MHz, DMSO-d₆) δ 8.43 (s, 1H), 7.60 (dd, J=9.9, 8.2 Hz, 1H), 7.40-7.23 (m, 5H), 6.48 (dd, J=8.2, 1.9 Hz, 1H), 5.17 (s, 2H), 4.19 (q, J=7.2 Hz, 2H), 1.41 (t, J=7.3 Hz, 3H), 0.73 (td, J=6.8, 4.6 Hz, 2H), 0.58-0.37 (m, 2H). LRMS m/z: 381.4 [M+H]⁺. Intermediate 30A: ¹H NMR (400 MHz, DMSO-d₆) δ 8.51 (s, 1H), 7.81 (t, J=8.3 Hz, 1H), 7.45-7.25 (m, 5H), 7.02 (s, 1H), 6.24 (dd, J=8.0, 2.9 Hz, 1H), 5.18 (s, 2H), 4.21 (q, J=7.2 Hz, 2H), 1.41 (t, J=7.3 Hz, 3H), 0.67 (td, J=6.9, 4.7 Hz, 2H), 0.43-0.35 (m, 2H). LRMS m/z: 381.3 [M+H]⁺.

The following intermediate compounds were prepared by an analogous procedure to that used for intermediate 30A, with variations to the reaction time and/or temperature as indicated. Intermediates 30N, 30O, 30P were prepared by conventional heating.

| | | | | | Reaction |
|---|---|---|---|---|---|
| Preparatory | | | | LRMS m/z | time/ |
| Example | Structure | Name | ¹H NMR δ (DMSO-d₆) | TLC R_{f} | Temp. (° C.) |
| 30B | 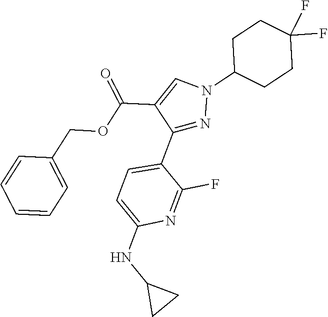 [SMILES: O=C(OCc1ccccc1)c1cn(C2CCC(F)(F)CC2)nc1-c1ccc(NC2CC2)nc1F] | Benzyl 3-[6- (cyclopropyl- amino)-2- fluoropyridin- 3-yl]-1-(4,4- difluorocyclo- hexyl) pyrazole-4- carboxylate | (400 MHz) 8.47 (s, 1H), 7.60 (dd, J = 9.9, 8.2 Hz, 1H), 7.41-7.24 (m, 5H), 6.48 (dd, J = 8.3, 1.9 Hz, 1H), 5.16 (s, 2H), 4.47 (s, 1H), 2.20-1.87 (m, 7H), 0.73 (td, J = 6.8, 4.6 Hz, 2H), 0.52-0.37 (m, 2H) | 471.4 [M + H]⁺ | 30 min 130° C. |
| 30C | 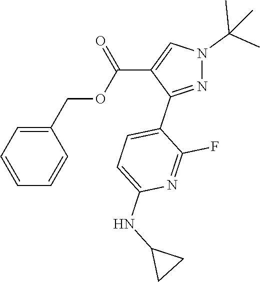 [SMILES: CC(C)(C)n1cc(C(=O)OCc2ccccc2)c(-c2ccc(NC3CC3)nc2F)n1] | Benzyl 1-tert- butyl-3-[6- (cyclopropyl- amino)-2- fluoropyridin- 3- yl]pyrazole- 4-carboxylate | (400 MHz) 8.37 (s, 1H), 7.59 (dd, J = 9.9, 8.2 Hz, 1H), 7.42-7.24 (m, 6H), 6.48 (dd, J = 8.2, 1.9 Hz, 1H), 5.17 (s, 2H), 1.56 (s, 9H), 0.73 (td, J = 6.8, 4.6 Hz, 2H), 0.54-0.36 (m, 2H) | 409.3 [M + H]⁺ | 30 min 130° C. |
| 30D | 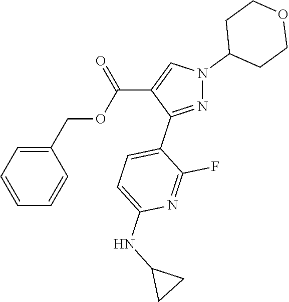 [SMILES: O=C(OCc1ccccc1)c1cn(C2CCOCC2)nc1-c1ccc(NC2CC2)nc1F] | Benzyl 3-[6- (cyclopropyl- amino)-2- fluoropyridin- 3-yl]-1- (oxan-4- yl)pyrazole- 4-carboxylate | (400 MHz) 8.46 (s, 1H), 7.60 (dd, J = 10.0, 8.2 Hz, 1H), 7.44-7.22 (m, 6H), 6.48 (dd, J = 8.2, 1.9 Hz, 1H), 5.17 (s, 2H), 4.56- 4.39 (m, 1H), 4.04-3.88 (m, 2H), 3.55-3.39 (m, 2H), 2.04-1.92 (m, 4H), 0.79-0.64 (m, 2H), 0.51- 0.33 (m, 2H) | 437.5 [M + H]⁺ | 30 min 130° C. |
| 30E | 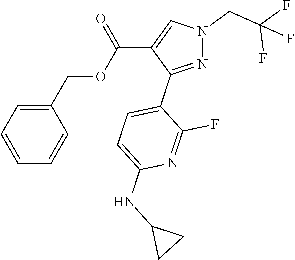 [SMILES: O=C(OCc1ccccc1)c1cn(CC(F)(F)F)nc1-c1ccc(NC2CC2)nc1F] | Benzyl 3-[6- (cyclopropyl- amino)-2- fluoropyridin- 3-yl]-1- (2,2,2- trifluoroethyl) pyrazole-4- carboxylate | — | 435.4 [M + H]⁺; TLC (1:1 Heptane: EtOAc) R_{f} = 0.49. | 30 min 130° C. |
| 30F | 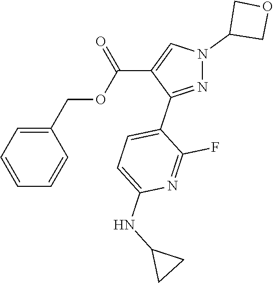 [SMILES: O=C(OCc1ccccc1)c1cn(C2COC2)nc1-c1ccc(NC2CC2)nc1F] | Benzyl 3-[6- (cyclopropyl- amino)-2- fluoropyridin- 3-yl]-1- (oxetan-3- yl)pyrazole- 4-carboxylate | (400 MHz) 8.55 (s, 1H), 7.66 (dd, J = 9.9, 8.2 Hz, 1H), 7.43-7.25 (m, 6H), 6.50 (dd, J = 8.2, 1.9 Hz, 1H), 5.64 (tt, J = 7.5, 6.3 Hz, 1H), 5.18 (s, 2H), 5.00- 4.83 (m, 4H), 0.73 (td, J = 6.8, 4.6 Hz, 2H), 0.55-0.37 (m, 2H). | 409.7 [M + H]⁺ | 30 min 130° C. |
| 30G | 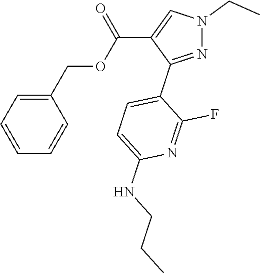 [SMILES: CCCNc1ccc(-c2nn(CC)cc2C(=O)OCc2ccccc2)c(F)n1] | Benzyl 1- ethyl-3-[2- fluoro-6- (propylamino) pyridin-3- yl]pyrazole- 4-carboxylate | (400 MHz) 8.41 (s, 1H), 7.50 (dd, J = 10.0, 8.2 Hz, 1H), 7.38-7.26 (m, 5H), 7.10 (t, J = 5.5 Hz, 1H), 6.36 (dd, J = 8.3, 1.9 Hz, 1H), 5.17 (s, 2H), 4.18 (q, J = 7.2 Hz, 2H), 3.17 (td, J = 7.0, 5.6 Hz, 2H), 1.62-1.47 (m, 2H), 1.40 (t, J = 7.3 Hz, 3H), 0.93 (t, J = 7.4 Hz, 3H). | 383.6 [M + H]⁺ | 40 min 120° C. |
| 30H | 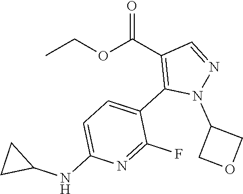 | Ethyl 5-[6- (cyclopropyl- amino)-2- fluoropyridin- 3-yl]-1- (oxetan-3- yl)pyrazole- 4-carboxylate | — | 375.5 [M + H]⁺; TLC (1:1 Heptane: EtOAc) R_{f} = 0.44. | 30 min 100° C. |
| 30I | 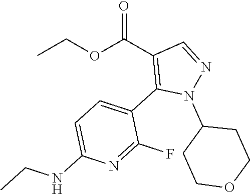 [SMILES: CCNc1ccc(-c2c(C(=O)OCC)cnn2C2CCOCC2)c(F)n1] | Ethyl 5-[6- (ethylamino)- 2- fluoropyridin- 3-yl]-1- (oxan-4- yl)pyrazole- 4-carboxylate | — | 363.0 [M + H]⁺; TLC (1:1 Heptane: EtOAc) R_{f} = 0.53. | 15 min 100° C. |
| 30J | 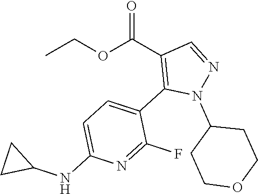 [SMILES: CCOC(=O)c1cnn(C2CCOCC2)c1-c1ccc(NC2CC2)nc1F] | Ethyl 5-[6- (cyclopropyl- amino)-2- fluoropyridin- 3-yl]-1- (oxan-4- yl)pyrazole- 4-carboxylate | — | 375.5 [M + H]⁺; TLC (1:1 Heptane: EtOAc) R_{f} = 0.44. | 30 min 100° C. |
| 30K | 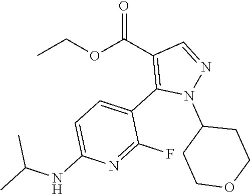 [SMILES: CCOC(=O)c1cnn(C2CCOCC2)c1-c1ccc(NC(C)C)nc1F] | Ethyl 5-[2- fluoro-6- (propan-2- ylamino) pyridin-3-yl]- 1-(oxan-4- yl)pyrazole- 4-carboxylate | — | 377.8 [M + H]⁺; TLC (1:1 Heptane: EtOAc) R_{f} = 0.55. | 30 min 110° C. |
| 30L | 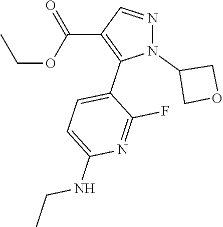 [SMILES: CCNc1ccc(-c2c(C(=O)OCC)cnn2C2COC2)c(F)n1] | Ethyl 5-[6- (ethylamino)- 2- fluoropyridin- 3-yl]-1- (oxetan-3- yl)pyrazole- 4-carboxylate | (700 MHz) 8.10 (s, 1H), 7.50-7.33 (m, 2H), 6.43 (dd, J = 8.3, 1.7 Hz, 1H), 5.33 (t, J = 6.9 Hz, 1H), 5.08- 4.75 (m, 4H), 4.09 (q, J = 7.1 Hz, 2H), 1.21-1.07 (m, 5H). | 334.8 [M + H]⁺ | 30 min 100° C. |
| 30M | 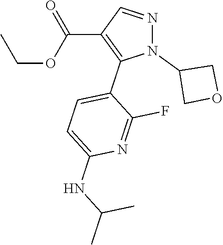 [SMILES: CCOC(=O)c1cnn(C2COC2)c1-c1ccc(NC(C)C)nc1F] | Ethyl 5-[2- fluoro-6- (propan-2- ylamino) pyridin-3-yl]- 1-(oxetan-3- yl)pyrazole- 4-carboxylate | (700 MHz) δ 8.10 (s, 1H), 7.45-7.22 (m, 2H), 6.41 (dd, J = 8.4, 1.7 Hz, 1H), 5.36-5.30 (m, 1H), 5.06- 4.72 (m, 4H), 4.19-4.13 (m, 1H), 4.09 (q, J = 7.0 Hz, 2H), 1.19-1.11 (m, 9H). | 349.3 [M + H]⁺ | 30 min 100° C. |
| 30N | 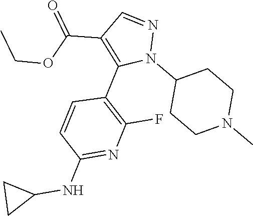 [SMILES: CCOC(=O)c1cnn(C2CCN(C)CC2)c1-c1ccc(NC2CC2)nc1F] | Ethyl 5-[6- (cyclopropyl- amino)-2- fluoropyridin- 3-yl]-1-(1- methylpiperidin- 4- yl)pyrazole- 4-carboxylate | (400 MHz) δ 7.97 (s, 1H), 7.66-7.47 (m, 2H), 6.57 (d, J = 8.3 Hz, 1H), 4.12-3.99 (m, 2H), 3.89-3.76 (m, 1H), 2.81 (d, J = 11.5 Hz, 2H), 2.14 (s, 3H), 2.11- 1.99 (m, 2H), 1.94-1.81 (m, 2H), 1.81-1.73 (m, 1H), 1.73-1.57 (m, 1H), 1.10 (t, J = 7.1 Hz, 3H), 0.80-0.66 (m, 2H), 0.53- 0.38 (m, 2H) | 388.3 [M + H]⁺ | 1 h 100° C. |
| 30O | 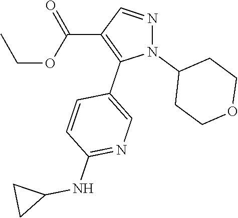 [SMILES: CCOC(=O)c1cnn(C2CCOCC2)c1-c1ccc(NC2CC2)nc1] | Ethyl 5-[6- (cyclopropyl- amino)pyridin- 3-yl]-1- (oxan-4- yl)pyrazole- 4-carboxylate | (600 MHz) δ 8.00 (dd, J = 2.5, 0.8 Hz, 1H), 7.97 (s, 1H), 7.49 (dd, J = 8.6, 2.4 Hz, 1H), 7.16 (d, J = 2.6 Hz, 1H), 6.68 (d, J = 8.6 Hz, 1H), 4.25-4.16 (m, 1H), 4.07 (q, J = 7.1 Hz, 2H), 3.94-3.84 (m, 2H), 3.35- 3.32 (m, 2H), 2.61-2.54 (m, 1H), 2.14-2.03 (m, 2H), 1.81-1.70 (m, 2H), 1.12 (t, J = 7.1 Hz, 3H), 0.77-0.69 (m, 2H), 0.49- 0.42 (m, 2H). | 357.1 [M + H]⁺ | 18 h 100° C. |
| 30P | 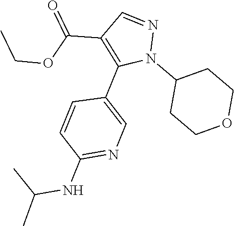 [SMILES: CCOC(=O)c1cnn(C2CCOCC2)c1-c1ccc(NC(C)C)nc1] | Ethyl 1- (oxan-4-yl)- 5-[6-(propan- 2- ylamino) pyridin-3- yl]pyrazole- 4-carboxylate | (600 MHz) δ 7.95-7.89 (m, 2H), 7.33 (dd, J = 8.6, 2.5 Hz, 1H), 6.73 (d, J = 7.6 Hz, 1H), 6.49 (dd, J = 8.8, 0.8 Hz, 1H), 4.22-4.12 (m, 1H), 4.09-3.96 (m, 3H), 3.90-3.82 (m, 2H), 2.10- 1.99 (m, 2H), 1.76-1.69 (m, 2H), 1.14 (d, J = 6.5 Hz, 6H), 1.08 (t, J = 7.1 Hz, 3H) | 359.2 [M + H]⁺ | 18 h 100° C. |

### 31A 5-(6-(Cyclopropylaminino)-2-fluoropyridin-3-yl)-1-ethyl-1H-pyrazole-4-carboxylic acid

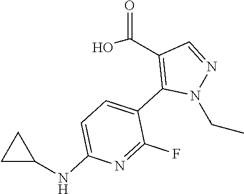 [SMILES: CC(C)n1ncc(C(=O)O)c1-c1ccc(NC2CC2)nc1F]

5 To a solution of intermediate 21C (231 mg, 0.730 mmol) in 1:1:1 MeOH:THF:water (3 mL) was added LiOH (140 mg, 5.850 mmol). The reaction mixture was heated to 50° C. and stirred for 2 h. HCl (1 M aq, 5 mL) and CH₂Cl₂ (10 mL) were added, the phases separated, and the aqueous phase extracted with 10% IPA/CHCl₃ (5×10 mL). The combined organic phases were washed with brine (20 mL), dried (MgSO₄) and the solvent was removed under reduced pressure to afford a pale yellow solid (195 mg, 93%). LCMS (method A) m/z 291.2 [M+H]⁺ at 1.03 min ¹H NMR (500 MHz, DMSO-d₆) δ 12.10 (s, 1H), 7.90 (s, 1H), 7.63-7.56 (m, 1H), 7.54 (d, J=2.6 Hz, 1H), 6.56 (d, J=8.3 Hz, 1H), 3.95-3.90 (m, 2H), 2.59-2.54 (m, 1H), 1.26 (t, J=7.2 Hz, 3H), 0.78-0.71 (m, 2H), 0.51-0.45 (in, 2H).

The following intermediate compounds were prepared by the same general procedure.

| Preparatory | | | ¹H NMR δ | LCMS |
|---|---|---|---|---|
| Example | Structure | Name | (500 MHz, DMSO-d₆) | m/z |
| 31B | 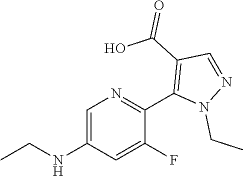 [SMILES: CCNc1cnc(-c2c(C(=O)O)cnn2CC)c(F)c1] | 1-Ethyl-5-[5- (ethylamino)-3- fluoropyridin-2- yl]pyrazole-4- carboxylic acid | δ 7.94-7.90 (m, 1H), 7.87 (s, 1H), 6.84 (dd, J = 12.6, 2.3 Hz, 1H), 6.80- 6.42 (m, 1H), 4.00 (q, J = 7.2 Hz, 2H), 3.13 (q, J = 7.2 Hz, 2H), 1.23 (t, J = 7.2 Hz, 3H), 1.19 (t, J = 7.1 Hz, 3H) | method A 279.1 [M + H]⁺ at 0.95 min |
| 31C | 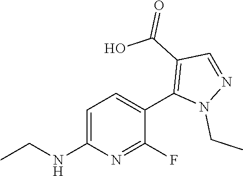 [SMILES: CCNc1ccc(-c2c(C(=O)O)cnn2CC)c(F)n1] | 1-Ethyl-5-(6- (ethylamino)-2- fluoropyridin-3-yl)- 1H-pyrazole-4- carboxylic acid | δ 12.06 (s, 1H), 7.89 (s, 1H), 7.51- 7.44 (m, 1H), 7.33-7.28 (m, 1H), 6.42 (dd, J = 8.3, 1.9 Hz, 1H), 3.91 (q, J = 7.2 Hz, 2H), 3.30-3.21 (m, 2H), 1.25 (t, J = 7.2 Hz, 3H), 1.15 (t, J = 7.2 Hz, 3H). | method A 279.2 [M + H]⁺ at 1.01 min. |
| 31D | 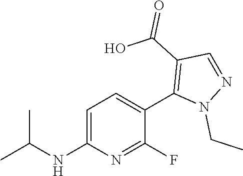 [SMILES: CCn1ncc(C(=O)O)c1-c1ccc(NC(C)C)nc1F] | 1-Ethyl-5-(2-fluoro- 6- (isopropylamino) pyridin-3-yl)-1H- pyrazole-4-carboxylic acid | δ 12.07 (s, 1H), 7.89 (s, 1H), 7.46 (dd, J = 10.0, 8.3 Hz, 1H), 7.21 (d, J = 7.5 Hz, 1H), 6.41 (dd, J = 8.3, 1.8 Hz, 1H), 3.98-3.89 (m, 3H), 1.26 (t, J = 7.2 Hz, 3H), 1.17 (d, J = 6.4 Hz, 6H). | method A 293.1 [M + H]⁺ at 1.13 min. |
| 31E | 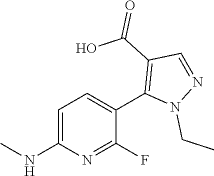 [SMILES: CCn1ncc(C(=O)O)c1-c1ccc(NC)nc1F] | 1-Ethyl-5-(2-fluoro- 6- (methylamino)pyridin- 3-yl)-1H-pyrazole-4- carboxylic acid | 12.09 (s, 1H), 7.89 (s, 1H), 7.53-7.46 (m, 1H), 7.30-7.25 (m, 1H), 6.43 (dd, J = 8.3, 1.9 Hz, 1H), 3.96-3.87 (m, 2H), 2.79 (d, J = 4.8 Hz, 3H), 1.25 (t, J = 7.2 Hz, 3H). | method A m/z 265.1 [M + H]⁺ at 0.88 min |
| 31F | 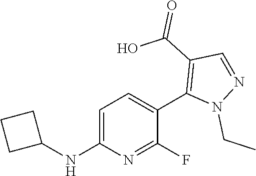 [SMILES: CCn1ncc(C(=O)O)c1-c1ccc(NC2CCC2)nc1F] | 5-(6- (Cyclobutylamino)-2- fluoropyridin-3-yl)-1- ethyl-1H-pyrazole-4- carboxylic acid | 12.09 (s, 1H), 7.89 (s, 1H), 7.60 (d, J = 7.2 Hz, 1H), 7.54-7.45 (m, 1H), 6.38 (dd, J = 8.3, 1.8 Hz, 1H), 4.22 (br s, 1H), 3.96-3.84 (m, 2H), 2.33- 2.25 (m, 2H), 1.98-1.86 (m, 2H), 1.75-1.61 (m, 2H), 1.25 (t, J = 7.2 Hz, 3H). | method A 305.1 [M + H]⁺ at 1.18 min. |
| 31G | 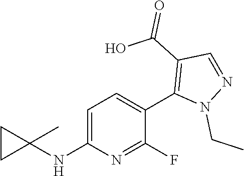 [SMILES: CCn1ncc(C(=O)O)c1-c1ccc(NC2(C)CC2)nc1F] | 1-Ethyl-5-[2-fluoro- 6-[(1- methylcyclopropyl) amino]pyridin-3- yl]pyrazole-4- carboxylic acid | 12.10 (s, 1H), 7.90 (s, 1H), 7.62- 7.55 (m, 2H), 6.55-6.50 (m, 1H), 3.92 (q, J = 7.2 Hz, 2H), 1.35 (s, 3H), 1.26 (t, J = 7.2 Hz, 3H), 0.74-0.65 (m, 4H). | method A 305.1 [M+ H]⁺ at 1.14 min. |
| 31H | 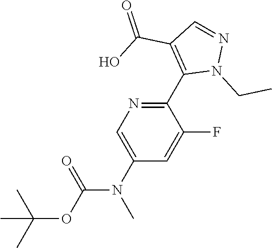 [SMILES: CCn1ncc(C(=O)O)c1-c1ncc(N(C)C(=O)OC(C)(C)C)cc1F] | 5-(5-((tert- Butoxycarbonyl) (methyl)amino)-3- fluoropyridin-2-yl)-1- ethyl- 1H-pyrazole-4- carboxylic acid | 12.32 (s, 1H), 8.66-8.62 (m, 1H), 7.99-7.92 (m, 1H), 7.95 (s, 1H), 4.03 (q, J = 7.2 Hz, 2H), 1.46 (s, 9H), 1.26 (t, J = 7.2 Hz, 3H). | method A 365.1 [M + H]⁺ at 1.25 min. |
| 31I | 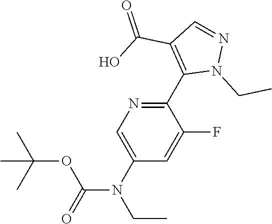 [SMILES: CCN(C(=O)OC(C)(C)C)c1cnc(-c2c(C(=O)O)cnn2CC)c(F)c1] | 5-(5-((tert- Butoxycarbonyl) (ethyl)amino)-3- fluoropyridin-2-yl)-1- ethyl-1H-pyrazole-4- carboxylic acid | 12.33 (s, 1H), 8.56 (dd, J = 2.1, 1.1 Hz, 1H), 7.95 (s, 1H), 7.95-7.91 (m, 1H), 4.03 (q, J = 7.2 Hz, 2H), 3.76 (q, J = 7.1 Hz, 2H), 1.43 (s, 9H), 1.25 (t, J = 7.2 Hz, 3H), 1.16 (t, J = 7.1 Hz, 3H). | method A 379.2 [M + H]⁺ at 1.33 min). |
| 31J | 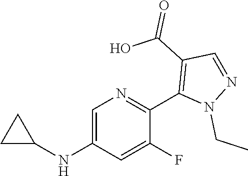 [SMILES: CCn1ncc(C(=O)O)c1-c1ncc(NC2CC2)cc1F] | 5-[5- (Cyclopropylamino)- 3-fluoropyridin-2-yl]- 1-ethylpyrazole-4- carboxylic acid | 8.01-7.96 (m, 1H), 7.88 (s, 1H), 7.08 (s, 1H), 6.96 (dd, J = 12.2, 2.2 Hz, 1H), 4.00 (q, J = 7.2 Hz, 2H), 2.49- 2.41 (m, 1H), 1.24 (t, J = 7.2 Hz, 3H), 0.81-0.74 (m, 2H), 0.49-0.43 (m, 2H). | method A 291.1 [M + H]⁺ at 1.01 min. |
| 31K | 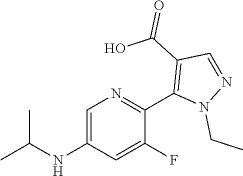 [SMILES: CCn1ncc(C(=O)O)c1-c1ncc(NC(C)C)cc1F] | 1-Ethyl-5-[3-fluoro- 5-(propan-2- ylamino)pyridin-2- yl]pyrazole-4- carboxylic acid | 7.92-7.89 (m, 1H), 7.87 (s, 1H), 6.84 (dd, J = 12.7, 2.3 Hz, 1H), 6.52 (s, 1H), 4.00 (q, J = 7.2 Hz, 2H), 3.67- 3.59 (m, 1H), 1.24 (t, J = 7.2 Hz, 3H), 1.17 (d, J = 6.3 Hz, 6H). | method A 293.2 [M + H]⁺ at 1.05 min. |
| 31L | 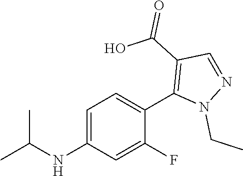 [SMILES: CCn1ncc(C(=O)O)c1-c1ccc(NC(C)C)cc1F] | 1-Ethyl-5-[2-fluoro- 4-(propan-2- ylamino)phenyl] pyrazole-4-carboxylic acid | 11.97 (s, 1H), 7.88 (s, 1H), 7.03 (t, J = 8.5 Hz, 1H), 6.45 (dd, J = 8.5, 2.2 Hz, 1H), 6.39 (dd, J = 13.2, 2.2 Hz, 1H), 6.15 (d, J = 8.0 Hz, 1H), 3.93-3.86 (m, 2H), 3.61-3.53 (m, 1H), 1.25- 1.21 (m, 3H), 1.16 (d, J = 6.3 Hz, 6H). | method A 292.2 [M + H]⁺ at 1.21 min. |
| 31M | 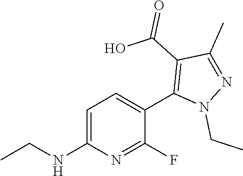 [SMILES: CCNc1ccc(-c2c(C(=O)O)c(C)nn2CC)c(F)n1] | 1-Ethyl-5-(6- (ethylamino)-2- fluoropyridin-3-yl)-3- methyl-1H-pyrazole- 4-carboxylic acid | 11.90 (s, 1H), 7.47-7.40 (m, 1H), 7.27-7.23 (m, 1H), 6.40 (dd, J = 8.3, 1.9 Hz, 1H), 3.82 (q, J = 7.2 Hz, 2H), 3.27-3.20 (m, 2H), 2.34 (s, 3H), 1.22 (t, J = 7.2 Hz, 3H), 1.15 (t, J = 7.2 Hz, 3H). | method A 293.1 [M + H]⁺ at 1.04 min. |
| 31N | 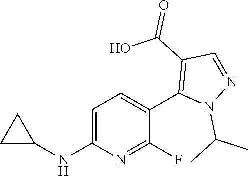 [SMILES: CC(C)n1ncc(C(=O)O)c1-c1ccc(NC2CC2)nc1F] | 5-[6- (Cyclopropylamino)- 2-fluoropyridin-3-yl]- 1-propan-2- ylpyrazole-4- carboxylic acid | Not available | method C, 305.1 [M + H]⁺ at 1.15 min. |

### 32A 5-[6-(Cyclopropylamino)-2-fluoropyridin-3-yl]-1-(oxan-4-yl)pyrazole-4-carboxylic acid

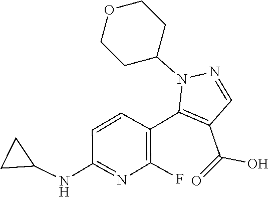

A solution of intermediate 30J (56 mg, 0.15 mmol) and LiOH (1 M aq., 1.2 mL, 1.2 mmol) in THF:MeOH (1:1; 6 mL) was heated at 55° C. for 4 h. The volatiles were removed under reduced pressure, and the residue acidified to pH≈2 with HCl (1 M aq.) then extracted with EtOAc (3×10 mL). The combined organic extracts were washed with water and brine (10 mL each), dried (MgSO₄), and the solvent was removed under reduced pressure to afford a white solid (50 mg, 970). ¹H NMR (400 MHz, DMSO-d₆) δ 12.15 (s, 1H), 7.95 (s, 1H), 7.66-7.51 (m, 2H), 6.57 (d, J=8.2 Hz, 1H), 4.21-4.07 (m, 1H), 3.95-3.81 (i, 2H), 2.16-2.04 (m, 2H), 1.85-1.63 (i, 2H), 0.86-0.70 (m, 2H), 0.64-0.41 (i, 2H). LCMS (method C), m/z 347 [M+H]⁺ at 1.60 min.

The following intermediate compounds were prepared by the same general procedure.

| Preparatory | | | | LRMS/ |
|---|---|---|---|---|
| Example | Structure | Name | ¹H NMR δ | LCMS m/z |
| 32B | 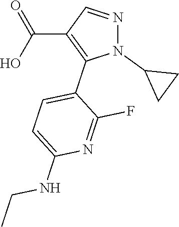 [SMILES: CCNc1ccc(-c2c(C(=O)O)cnn2C2CC2)c(F)n1] | 1-Cyclopropyl- 5-[6- (ethylamino)-2- fluoropyridin-3- yl]pyrazole-4- carboxylic acid | (400 MHz, DMSO-d₆) δ 12.16 (s, 1H), 7.81 (s, 1H), 7.56 (dd, J = 10.0, 8.3 Hz, 1H), 7.35-7.23 (m, 1H), 6.43 (dd, J = 8.4, 2.0 Hz, 1H), 3.45 (dt, J = 7.3, 3.5 Hz, 1H), 3.23-3.21 (m, 2H), 1.15 (t, J = 7.2 Hz, 3H), 1.01-0.93 (m, 2H), 0.91-0.81 (m, 2H). | LRMS 291.1 [M + H]⁺ |
| 32C | 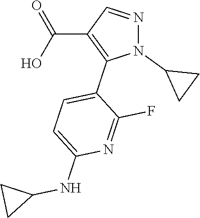 [SMILES: O=C(O)c1cnn(C2CC2)c1-c1ccc(NC2CC2)nc1F] | 1-Cyclopropyl- 5-[6- (cyclopropyl- amino)-2- fluoropyridin-3- yl]pyrazole-4- carboxylic acid | (400 MHz, CDCl₃) δ 7.97 (s, 1H), 7.76-7.61 (m, 1H), 7.56 (d, J = 7.9 Hz, 1H), 5.51 (s, 1H), 3.43 (dt, J = 7.5, 3.9 Hz, 1H), 2.88-2.80 (m, 2H), 1.29-1.21 (m, 2H), 0.94-0.85 (m, 4H), 0.66-0.60 (m, 2H). | LRMS 303.3 [M + H]⁺ |
| 32D | 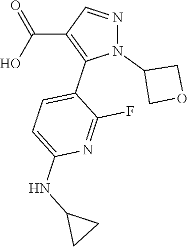 [SMILES: O=C(O)c1cnn(C2COC2)c1-c1ccc(NC2CC2)nc1F] | 5-[6- (Cyclopropyl- amino)-2- fluoropyridin-3- yl]-1-(oxetan-3- yl)pyrazole-4- carboxylic acid | (400 MHz, DMSO-d₆) δ 12.40 (s, 1H), 8.08 (s, 1H), 7.61 (d, J = 2.7 Hz, 1H), 7.54 (t, J = 9.1 Hz, 1H), 6.62- 6.48 (m, 1H), 5.37-5.25 (m, 1H), 4.97-4.74 (m, 4H), 2.62-2.53 (m, 1H), 0.79-0.72 (m, 2H), 0.52-0.45 (m, 2H). | LRMS 319.2 [M + H]⁺ |
| 32E | 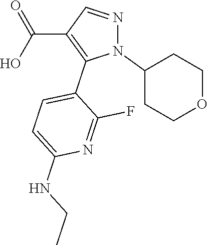 [SMILES: CCNc1ccc(-c2c(C(=O)O)cnn2C2CCOCC2)c(F)n1] | 5-[6- (Ethylamino)-2- fluoropyridin-3- yl]-1-(oxan-4- yl)pyrazole-4- carboxylic acid | (400 MHz, DMSO-d₆) δ 12.14 (s, 1H), 7.94 (s, 1H), 7.48 (dd, J = 10.0, 8.2 Hz, 1H), 7.32 (t, J = 5.4 Hz, 1H), 6.43 (dd, J = 8.3, 2.0 Hz, 1H), 4.18- 4.04 (m, 1H), 3.96-3.84 (m, 2H), 3.30-3.19 (m, 3H), 2.15-2.01 (m, 2H), 1.81-1.59 (m, 2H), 1.17 (t, J = 7.2 Hz, 3H). | LRMS 335.2 [M + H]⁺ |
| 32F | 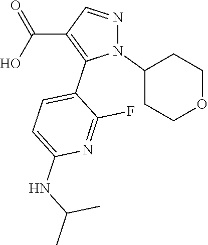 [SMILES: CC(C)Nc1ccc(-c2c(C(=O)O)cnn2C2CCOCC2)c(F)n1] | 5-[2-Fluoro-6- (propan-2- ylamino)pyridin- 3-yl]-1-(oxan- 4-yl)pyrazole-4- carboxylic acid | (400 MHz, DMSO-d₆) δ 12.15 (s, 1H), 7.95 (s, 1H), 7.47 (dd, J = 10.1, 8.3 Hz, 1H), 7.24 (d, J = 7.5 Hz, 1H), 6.42 (dd, J = 8.3, 2.0 Hz, 1H), 4.16- 4.06 (m, 1H), 3.99-3.85 (m, 3H), 2.15-2.00 (m, 2H), 1.86-1.60 (m, 2H), 1.18 (d, J = 6.4 Hz, 6H). | LRMS 349.2 [M + H]⁺ |
| 32G | 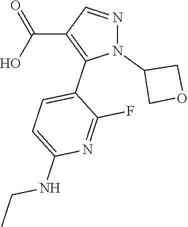 [SMILES: CCNc1ccc(-c2c(C(=O)O)cnn2C2COC2)c(F)n1] | 5-[2-Fluoro-6- (propan-2- ylamino)pyridin- 3-yl]-1- (oxetan-3- yl)pyrazole-4- carboxylic acid | (700 MHz, DMSO-d₆) 12.27 (s, 1H), 8.07 (s, 1H), 7.50-7.28 (m, 2H), 6.42 (dd, J = 8.3, 1.7 Hz, 1H), 5.34- 5.24 (m, 1H), 5.02-4.75 (m, 4H), 3.30-3.19 (m, 2H), 1.16 (t, J = 7.1 Hz, 3H). | LRMS 306.9 [M + H]⁺ |
| 32H | 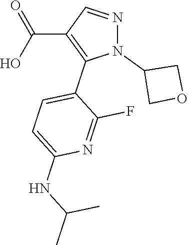 [SMILES: CC(C)Nc1ccc(-c2c(C(=O)O)cnn2C2COC2)c(F)n1] | 5-[2-Fluoro-6- (propan-2- ylamino)pyridin- 3-yl]-1- (oxetan-3- yl)pyrazole-4- carboxylic acid | (700 MHz, DMSO-d₆) 12.28 (s, 1H), 8.07 (s, 1H), 7.50-7.21 (m, 2H), 6.41 (dd, J = 8.3, 1.8 Hz, 1H), 5.34- 5.27 (m, 1H), 5.00-4.72 (m, 4H), 3.98-3.91 (m, 1H), 1.17 (dd, J = 6.5, 2.7 Hz, 6H). | LRMS 320.9 [M + H]⁺ |
| 32I | 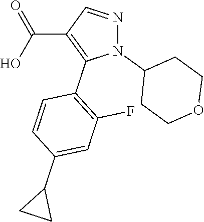 [SMILES: O=C(O)c1cnn(C2CCOCC2)c1-c1ccc(C2CC2)cc1F] | 5-(4- Cyclopropyl-2- fluorophenyl)-1- (oxan-4- yl)pyrazole-4- carboxylic acid | (400 MHz, DMSO-d₆) 12.15 (s, 1H), 7.98 (s, 1H), 7.29 (t, J = 7.9 Hz, 1H), 7.19-6.94 (m, 2H), 4.11-3.96 (m, 1H), 3.94-3.81 (m, 2H), 3.30-3.21 (m, 2H), 2.15-1.95 (m, 3H), 1.81- 1.73 (m, 1H), 1.68-1.58 (m, 1H), 1.10-0.97 (m, 2H), 0.88-0.76 (m, 2H). | LRMS 330.8 [M + H]⁺ |
| 32J | 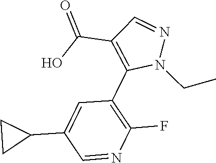 [SMILES: CCn1ncc(C(=O)O)c1-c1cc(C2CC2)cnc1F] | 5-(5- Cyclopropyl-2- fluoropyridin-3- yl)-1- ethylpyrazole-4- carboxylic acid | (500 MHz, DMSO-d₆) 12.29 (s, 1H), 8.20-8.15 (m, 1H), 7.96 (s, 1H), 7.74 (dd, J = 9.0, 2.6 Hz, 1H), 3.93 (q, J = 7.2 Hz, 2H), 2.09-2.00 (m, 1H), 1.25 (t, J = 7.1 Hz, 3H), 1.06- 0.98 (m, 2H), 0.83-0.76 (m, 2H) | LCMS method A 276.4 [M + H]⁺ at 1.15 min. |
| 32K | 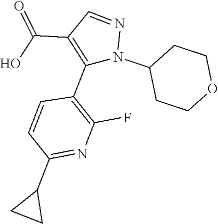 [SMILES: O=C(O)c1cnn(C2CCOCC2)c1-c1ccc(C2CC2)nc1F] | 5-(6- Cyclopropyl-2- fluoropyridin-3- yl)-1-(oxan-4- yl)pyrazole-4- carboxylic acid | (400 MHz, DMSO-d₆) 12.26 (s, 1H), 8.00 (s, 1H), 7.89 (dd, J = 9.8, 7.6 Hz, 1H), 7.43 (dd, J = 7.7, 1.9 Hz, 1H), 4.16-4.02 (m, 1H), 3.95-3.82 (m, 2H), 2.27-2.16 (m, 1H), 2.13- 1.99 (m, 2H), 1.90-1.78 (m, 1H), 1.69-1.59 (m, 1H), 1.11-1.03 (m, 2H), 1.04-0.92 (m, 2H). | LRMS 332.2 [M + H]⁺ |
| 32L | 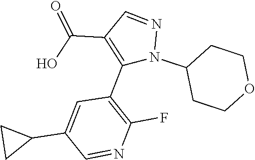 [SMILES: O=C(O)c1cnn(C2CCOCC2)c1-c1cc(C2CC2)cnc1F] | 5-(5- Cyclopropyl-2- fluoropyridin-3- yl)-1-(oxan-4- yl)pyrazole-4- carboxylic acid | (400 MHz, DMSO-d₆) 12.29 (s, 1H), 8.21-8.17 (m, 1H), 8.01 (s, 1H), 7.72 (dd, J = 9.0, 2.5 Hz, 1H), 4.22- 3.98 (m, 1H), 3.94-3.82 (m, 2H), 2.19-1.98 (m, 3H), 1.84 (d, J = 12.8 Hz, 1H), 1.65 (d, J = 12.9 Hz, 1H), 1.11-0.94 (m, 2H), 0.85-0.59 (m, 2H). | LRMS 332.1 [M + H]⁺ |
| 32M | 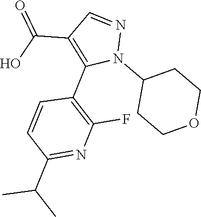 [SMILES: CC(C)c1ccc(-c2c(C(=O)O)cnn2C2CCOCC2)c(F)n1] | 5-(2-Fluoro-4- propan-2- ylphenyl)-1- (oxan-4- yl)pyrazole-4- carboxylic acid | (600 MHz, DMSO-d₆) 12.12 (s, 1H), 7.99 (s, 1H), 7.36 (t, J = 7.7 Hz, 1H), 7.29-7.10 (m, 2H), 4.05-3.96 (m, 1H), 3.93-3.75 (m, 2H), 3.32-3.22 (m, 2H), 3.06-2.95 (m, 1H), 2.13- 2.01 (m, 2H), 1.87-1.71 (m, 1H), 1.69-1.61 (m, 1H), 1.26 (d, J = 6.9 Hz, 6H). | LRMS 333.1 [M + H]⁺ |
| 32N | 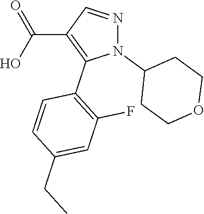 [SMILES: CCc1ccc(-c2c(C(=O)O)cnn2C2CCOCC2)c(F)c1] | 5-(4-Ethyl-2- fluorophenyl)-1- (oxan-4- yl)pyrazole-4- carboxylic acid | (600 MHz, DMSO-d₆) 12.12 (s, 1H), 7.99 (s, 1H), 7.35 (t, J = 7.7 Hz, 1H), 7.27-7.16 (m, 2H), 4.07-3.96 (m, 1H), 3.96-3.74 (m, 2H), 3.30-3.24 (m, 2H), 2.72 (q, J = 7.6 Hz, 2H), 2.22-2.02 (m, 2H), 1.83-1.72 (m, 1H), 1.72-1.54 (m, 1H), 1.25 (t, J = 7.6 Hz, 3H). | LRMS 319.1 [M + H]⁺ |
| 32O | 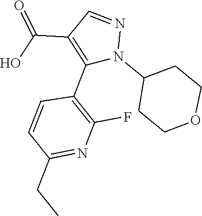 [SMILES: CCc1ccc(-c2c(C(=O)O)cnn2C2CCOCC2)c(F)n1] | 5-(6-Ethyl-2- fluoropyridin-3- yl)-1-(oxan-4- yl)pyrazole-4- carboxylic acid | (500 MHz, DMSO-d₆) 12.27 (s, 1H), 8.06-7.89 (m, 2H), 7.38 (dd, J = 7.6, 1.8 Hz, 1H), 4.16-4.01 (m, 1H), 3.91-3.79 (m, 2H), 2.86-2.76 (m, 2H), 2.14-2.02 (m, 2H), 1.82 (d, J = 12.9 Hz, 1H), 1.66 (d, J = 12.8 Hz, 1H), 1.28 (t, J = 7.5 Hz, 3H). | LRMS 320.3 [M + H]⁺ |
| 32P | 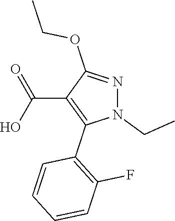 [SMILES: CCOc1nn(CC)c(-c2ccccc2F)c1C(=O)O] | 3-Ethoxy-1- ethyl-5-(2- fluorophenyl) pyrazole-4- carboxylic acid | (500 MHz, DMSO-d₆) 11.73 (s, 1H), 7.59-7.51 (m, 1H), 7.46-7.39 (m, 1H), 7.38-7.28 (m, 2H), 4.25 (q, J = 7.0 Hz, 2H), 3.79-3.67 (m, 2H), 1.35 (t, J = 7.0 Hz, 3H), 1.18 (t, J = 7.2 Hz, 3H). | LCMS method A 279.4 [M + H]⁺ at 1.15 min. |

### 33A 3-[6-(Cyclopropylamino)-2-fluoropyridin-3-yl]-1-(oxan-4-yl)pyrazole-4-carboxylic acid

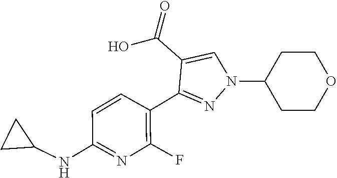 [SMILES: O=C(O)c1cn(C2CCOCC2)nc1-c1ccc(NC2CC2)nc1F]

Palladium hydroxide on carbon, (20 wt. % loading, 50% water; 34.9 mg, 0.22 mmol) was added to a solution of intermediate 35D (96 mg, 0.22 mmol) in EtOH (5 mL) in a pressure tube, the vessel purged successively with N₂ (5×) and hydrogen (5×), then stirred under H₂ at 40 psi for 70 min. The reaction was diluted with CH₂Cl₂ (15 mL) and EtOH (5 mL), filtered through a glass microfiber pad, washing with CH₂Cl₂ and the solvent removed under reduced pressure to afford a white solid (58 mg, 76%). ¹H NMR (400 MHz, DMSO-d₆) δ 12.14 (s, 1H), 8.31 (s, 1H), 7.60 (dd, J=9.9, 8.2 Hz, 1H), 7.27 (d, J=2.5 Hz, 1H), 6.49 (dd, J=8.2, 1.9 Hz, 1H), 4.44 (dq, J=10.4, 5.4 Hz, 1H), 3.96 (dt, J=11.7, 3.2 Hz, 2H), 3.45 (td, J=11.5, 3.5 Hz, 3H), 1.99 (q, J=4.4 Hz, 4H), 0.84-0.69 (m, 2H), 0.57-0.38 (m, 2H). LRMS m/z: 347.5 [M+H]⁺

The following intermediate compounds were prepared by an analogous procedure to that described for intermediate 33A.

| Preparatory | | | ¹H NMR δ | LRMS |
|---|---|---|---|---|
| examples | Structure | Name | (400 MHz, DMSO-d₆) | m/z |
| 33B | 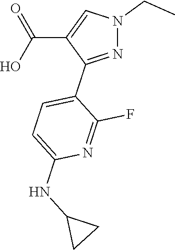 [SMILES: CCn1cc(C(=O)O)c(-c2ccc(NC3CC3)nc2F)n1] | 3-[6- (Cyclopropylamino)- 2-fluoropyridin-3-yl]- 1-ethylpyrazole-4- carboxylic acid | 12.17 (s, 1H), 8.28 (s, 1H), 7.60 (dd, J = 9.9, 8.2 Hz, 1H), 7.26 (d, J = 2.5 Hz, 1H), 6.49 (dd, J = 8.2, 1.9 Hz, 1H), 4.17 (q, J = 7.3 Hz, 2H), 1.40 (t, J = 7.3 Hz, 3H), 0.73 (td, J = 6.8, 4.6 Hz, 2H), 0.50- 0.39 (m, 2H). | 291.4 [M + H]⁺ |
| 33C | 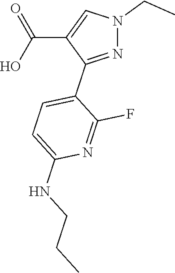 [SMILES: CCCNc1ccc(-c2nn(CC)cc2C(=O)O)c(F)n1] | 1-Ethyl-3-[2-fluoro-6- (propylamino)pyridin- 3-yl]pyrazole-4- carboxylic acid | — | 293.5 [M + H]⁺ |
| 33D | 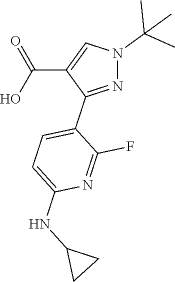 [SMILES: CC(C)(C)n1cc(C(=O)O)c(-c2ccc(NC3CC3)nc2F)n1] | 1-tert-butyl-3-[6- (cyclopropylamino)- 2-fluoropyridin-3- yl]pyrazole-4- carboxylic acid | 12.09 (s, 1H), 8.26 (s, 1H), 7.60 (dd, J = 9.9, 8.1 Hz, 1H), 7.25 (d, J = 2.6 Hz, 1H), 6.49 (dd, J = 8.2, 1.9 Hz, 1H), 1.55 (s, 9H), 0.72 (td, J = 6.8, 4.6 Hz, 2H), 0.52-0.33 (m, 2H). | 319.4 [M + H]⁺ |
| 33E | 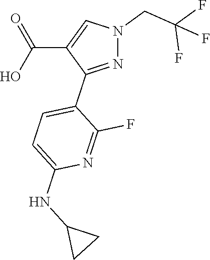 [SMILES: O=C(O)c1cn(CC(F)(F)F)nc1-c1ccc(NC2CC2)nc1F] | 3-[6- (Cyclopropylamino)- 2-fluoropyridin-3-yl]- 1-(2,2,2- trifluoroethyl)pyrazole- 4-carboxylic acid | 12.36 (s, 1H), 8.40 (s, 1H), 7.61 (dd, J = 9.9, 8.2 Hz, 1H), 7.33 (d, J = 2.6 Hz, 1H), 6.50 (dd, J = 8.2, 1.9 Hz, 1H), 5.21 (q, J = 9.0 Hz, 2H), 0.73 (td, J = 6.8, 4.6 Hz, 2H), 0.60- 0.31 (m, 2H). | 345.4 [M + H]⁺ |
| 33F | 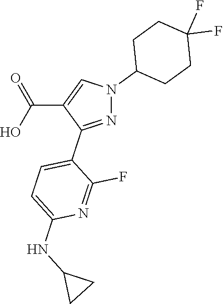 [SMILES: O=C(O)c1cn(C2CCC(F)(F)CC2)nc1-c1ccc(NC2CC2)nc1F] | 3-[6- (Cyclopropylamino)- 2-fluoropyridin-3-yl]- 1-(4,4- difluorocyclohexyl) pyrazole-4-carboxylic acid | 12.12 (s, 1H), 8.31 (s, 1H), 7.60 (dd, J = 9.9, 8.2 Hz, 1H), 7.27 (d, J = 2.5 Hz, 1H), 6.49 (dd, J = 8.2, 1.9 Hz, 1H), 4.44 (d, J = 6.5 Hz, 1H), 2.29-1.86 (m, 8H), 0.72 (dd, J = 6.8, 2.2 Hz, 2H), 0.45 (dd, J = 3.8, 2.4 Hz, 2H). | 381.5 [M + H]⁺ |
| 33G | 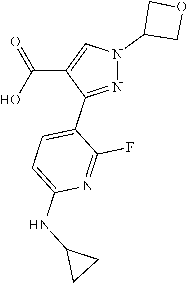 [SMILES: O=C(O)c1cn(C2COC2)nc1-c1ccc(NC2CC2)nc1F] | 3-[6- (Cyclopropylamino)- 2-fluoropyridin-3-yl]- 1-(oxetan-3- yl)pyrazole-4- carboxylic acid | 12.21 (s, 1H), 8.41 (s, 1H), 7.67 (dd, J = 9.9, 8.2 Hz, 1H), 7.32 (d, J = 2.6 Hz, 1H), 6.52 (dd, J = 8.2, 1.9 Hz, 1H), 5.73-5.51 (m, 1H), 5.03- 4.77 (m, 4H), 0.74 (td, J = 6.8, 4.6 Hz, 2H), 0.55-0.36 (m, 2H). | 319.5 [M + H]⁺ |

### 34A tert-Butyl (6-(1-Ethyl-4-(((S)-2-oxo-5 phenyl-2,3-dihydro-JH-benzo[e][1,4]diazepin-3-yl)carbamoyl)-1H-pyrazol-5-yl)-5-fluoropyridin-3-yl)(methyl)carbamate

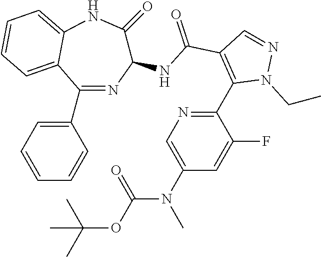 [SMILES: CCn1ncc(C(=O)N[C@H]2N=C(c3ccccc3)c3ccccc3NC2=O)c1-c1ncc(N(C)C(=O)OC(C)(C)C)cc1F]

HATU (28 mg, 0.074 mmol), NEt₃ (20 μL, 0.143 mmol), then (S)-3-amino-5-phenyl-1H-benzo[e][1,4]diazepin-2(3H)-one (18 mg, 0.072 mmol) were added to a solution of intermediate 31H in DMF (1 mL) and the reaction stirred at rt for 16 h. Water (20 mL) was added, and the resultant precipitate collected by filtration, washing with water. The precipitate was taken into CH₂Cl₂ (10 mL), passed through a phase separator containing brine (10 mL) and the solvent was removed under reduced pressure. Purification by flash chromatography [20-80% (10% MeOH/EtOAc)/isohexanes] afforded a white solid (35 mg, 81%). ¹H NMR (500 MHz, DMSO-d₆) δ 10.83 (s, 1H), 8.96 (d, J=7.8 Hz, 1H), 8.27 (s, 1H), 7.92 (t, J=1.9 Hz, 1H), 7.62 (ddd, J=8.6, 7.2, 1.6 Hz, 1H), 7.55-7.48 (m, 1H), 7.51-7.45 (m, 2H), 7.48-7.41 (m, 2H), 7.32-7.26 (m, 2H), 7.26-7.20 (m, 1H), 6.80 (dd, J=12.5, 2.3 Hz, 1H), 6.67 (s, 1H), 5.32 (d, J=7.8 Hz, 1H), 3.99 (q, J=7.2 Hz, 2H), 2.74 (d, J=5.0 Hz, 3H), 1.25 (t, J=7.2 Hz, 3H). LCMS (method B) m/z 498.2 [M+H]⁺ at 3.49 min.

### 34B tert-Butyl N-ethyl-N-[6-[2-ethyl-4-[[(3S)-2-oxo-5-phenyl-1,3-dihydro-1,4-benzodiazepin-3-yl]carbamoyl]pyrazol-3-yl]-5-fluoropyridin-3-yl]carbamate

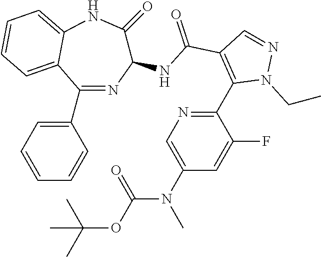 [SMILES: CCn1ncc(C(=O)N[C@H]2N=C(c3ccccc3)c3ccccc3NC2=O)c1-c1ncc(N(C)C(=O)OC(C)(C)C)cc1F]

Prepared by an analogous procedure to that described for intermediate 34A. ¹H NMR (500 MHz, DMSO-d₆) δ 10.83 (s, 1H), 8.96 (d, J=7.8 Hz, 1H), 8.27 (s, 1H), 7.93 (t, J=1.9 Hz, 1H), 7.62 (ddd, J=8.6, 7.1, 1.6 Hz, 1H), 7.55-7.48 (m, 1H), 7.51-7.45 (m, 2H), 7.48-7.41 (m, 2H), 7.32-7.26 (m, 2H), 7.26-7.20 (m, 1H), 6.81 (dd, J=12.6, 2.3 Hz, 1H), 6.65-6.59 (m, 1H), 5.32 (d, J=7.8 Hz, 1H), 3.99 (q, J=7.2 Hz, 2H), 3.15-3.06 (m, 2H), 1.25 (t, J=7.2 Hz, 3H), 1.17 (t, J=7.1 Hz, 3H). LCMS (method B) m/z 512.3 [M+H]⁺ at 3.86 min.

### 35A Ethyl 3-ethoxy-1-ethylpyrazole-4-carboxylate

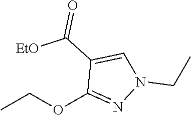

K₂CO₃ (1.7 g, 12.3 mmol) and then EtI (1 mL, 12.4 mmol) were added to a solution of ethyl 3-oxo-1,2-dihydropyrazole-4-carboxylate (0.9 g, 5.76 mmol) in MeCN (20 mL) and the reaction heated at 60° C. over the weekend. The reaction was cooled to rt and filtered, washing with MeCN (2×20 mL). The filtrate was concentrated under reduced pressure and purified by flash chromatography (0-50% MTBE/isohexanes) to afford a colourless oil (943 mg, 39%). A 1:1 mixture of the desired regioisomer with ethyl 5-ethoxy-1-ethylpyrazole-4-carboxylate was obtained, based upon the ¹H NMR 6 pyrazole CH signal. Used without further purification. ¹H NMR (500 MHz, CDCl₃) δ 7.71 (s, 1H), 4.40-4.32 (m, 2H), 4.32-4.24 (m, 2H), 4.07-3.98 (m, 2H), 1.51-1.43 (m, 6H), 1.38-1.30 (m, 3H). LCMS (Method A) 213.5 [M+H]⁺ at 1.08 min.

### 36A Ethyl 3-ethoxy-1-ethyl-5-(2-fluorophenyl)pyrazole-4-carboxylate

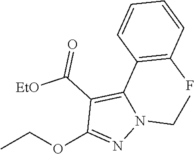 [SMILES: CCOC(=O)c1c(OCC)nn(CC)c1-c1ccccc1F]

A solution of crude intermediate 35A (300 mg, 1.41 mmol, ˜50% purity) in anhydrous THE (4 mL) was degassed with N₂. The solution was cooled to −78° C., and LDA (2.0 M in THF; 0.9 mL, 1.8 mmol) and then zinc (II) chloride (2.0 M in 2-Me THF; 0.9 mL, 1.8 mmol) added. The reaction mixture was warmed to rt and degassed with N₂. 1-Bromo-2-fluorobenzene (0.19 mL, 1.74 mmol), Pd-170; 38 mg, 0.06 mmol) and XPhos (27 mg, 0.06 mmol) were added, the reaction mixture evacuated, purged with N₂ and then heated to 70° C. for 5 h. 1 M aq. HCl (30 mL) and EtOAc (30 mL) were added, and separated aqueous phase extracted with EtOAc (2×10 mL). The combined organic phases were washed with brine (30 mL), dried (MgSO₄), and concentrated under reduced pressure. Purification by flash chromatography (0-50% MTBE/isohexanes) afforded a yellow gum (193 mg, 45%). ¹H NMR (500 MHz, CDCl₃) δ 7.50-7.42 (m, 1H), 7.33-7.26 (m, 1H), 7.26-7.20 (m, 1H), 7.20-7.13 (m, 1H), 4.38 (q, J=7.0 Hz, 2H), 4.10-4.02 (m, 2H), 3.86-3.77 (m, 2H), 1.47 (t, J=7.0 Hz, 3H), 1.30 (t, J=7.2 Hz, 3H), 1.02 (t, J=7.1 Hz, 3H). LCMS (Method A) 307.3 [M+H]⁺ at 1.50 min.

### 37A Ethyl 5-(5-chloro-2-fluoropyridin-3-yl)-1-ethylpyrazole-4-carboxylate

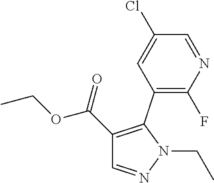 [SMILES: CCOC(=O)c1cnn(CC)c1-c1cc(Cl)cnc1F]

A solution of intermediate 1A (1 g, 5.95 mmol) in anhydrous THE (10 mL) was degassed with N₂. The solution was cooled to −78° C. and LDA (2.0 M in THF; 3.4 mL, 6.8 mmol) and then zinc (II) chloride (2.0 M in 2-Me THF; 3.4 mL, 6.8 mmol) added. The reaction was warmed to rt and degassed with N₂. Pd(PPh₃)₄ (350 mg, 0.3 mmol) and 3-bromo-5-chloro-2-fluoropyridine (1.3 g, 6.18 mmol) were added, and the reaction was evacuated and purged with N₂, then heated to 70° C. overnight. 1 M aq. HCl (50 mL) and EtOAc (50 mL) were added and the phases were separated. The aqueous phase was extracted with EtOAc (2×20 mL) and the combined organic phases washed with brine (100 mL), dried (MgSO₄), and concentrated under reduced pressure. Purification by flash chromatography (0-10% MTBE/isohexanes) afforded a yellow gum (1.2 g, 68%). ¹H NMR (500 MHz, CDCl₃) δ 8.33-8.28 (m, 1H), 8.05 (s, 1H), 7.83-7.77 (m, 1H), 4.21-4.13 (m, 2H), 4.04-3.98 (m, 2H), 1.41 (t, J=7.2 Hz, 3H), 1.19 (t, J=7.1 Hz, 3H). LCMS (Method A) 298.3 [M+H]⁺ at 1.36 min.

### 38A Ethyl (E)-2-(4-bromo-2-fluorobenzoyl)-3-(dimethylamino)prop-2-enoate

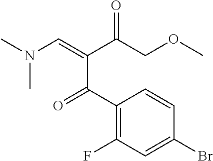

DMF (0.07 mL, 0.91 mmol) and thionyl chloride (1.67 mL, 22.8 mmol) were added to a suspension of 4-bromo-2-fluorobenzoic acid (1 g, 4.57 mmol) in toluene (13 mL). The mixture was heated at 110° C. for 2 h, cooled to rt and concentrated under reduced pressure. The residue was dissolved in THE (5 mL), NEt₃ (0.96 mL, 6.85 mmol) added, followed by dropwise addition of ethyl-N,N-dimethylaminoacrylate (0.65 mL, 4.57 mmol). The reaction was heated at reflux for 2 h, cooled to rt, and water and EtOAc (30 mL each) added. The mixture was extracted with EtOAc (3×30 ml), and the combined organics were washed with water (30 mL), brine (30 mL), dried (Na₂SO₄), and concentrated under reduced pressure. Purification by flash chromatography (10-100% EtOAc/heptane) afforded a yellow oil (1.16 g, 74%). ¹H NMR (700 MHz, DMSO-d₆) δ 7.75 (s, 1H), 7.53 (dd, J=9.8, 1.8 Hz, 1H), 7.43 (dd, J=8.2, 1.8 Hz, 1H), 7.37 (d, J=7.9 Hz, 1H), 3.87 (q, J=7.1 Hz, 2H), 2.77 (s, 3H), 0.89 (t, J=7.1 Hz, 3H). LRMS m/z 343.9/345.9 [M+H]⁺.

The following intermediate compounds were prepared using the same general procedure described for intermediate 38A.

| Preparatory | | | | LRMS m/z; |
|---|---|---|---|---|
| Example | Structure | Name | ¹H NMR (DMSO-d₆) δ | TLC R_{f} |
| 38B | 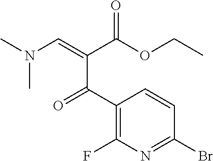 [SMILES: CCOC(=O)/C(=C/N(C)C)C(=O)c1ccc(Br)nc1F] | Ethyl (E)-2-(6- bromo-2- fluoropyridine-3- carbonyl)-3- (dimethylamino) prop-2-enoate | — | 345.0/347.0 [M + H]⁺; R_{f} = 0.24 (2:1 EtOAc/ heptane) |
| 38C | 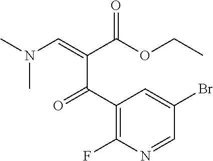 [SMILES: CCOC(=O)/C(=C/N(C)C)C(=O)c1cc(Br)cnc1F] | Ethyl (E)-2-(5- bromo-2- fluoropyridine-3- carbonyl)-3- (dimethylamino) prop-2-enoate | — | 345.0/347.0 [M + H]⁺; R_{f} = 0.38 (2:1 EtOAc/ heptane) |
| 38D | 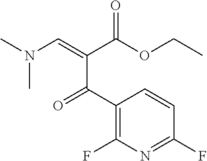 [SMILES: CCOC(=O)/C(=C/N(C)C)C(=O)c1ccc(F)nc1F] | Ethyl (E)-2-(2,6- difluoropyridine- 3-carbonyl)-3- (dimethylamino) prop-2-enoate | (700 MHz) 8.18 (dt, J = 9.2, 8.0 Hz, 1H), 7.86 (s, 1H), 7.20 (dd, J = 8.1, 2.3 Hz, 1H), 3.91 (q, J = 7.1 Hz, 2H), 3.35 (s, 3H), 2.79 (s, 3H), 0.93 (t, J = 7.1Hz, 3H). | 284.9 [M + H]⁺ |
| 38E | 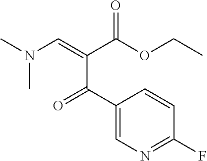 [SMILES: CCOC(=O)/C(=C/N(C)C)C(=O)c1ccc(F)nc1] | Ethyl (E)-3- (dimethylamino)- 2-(6- fluoropyridine-3- carbonyl)prop-2- enoate | (400 MHz) 7.75 (s, 1H), 7.53 (dd, J = 9.8, 1.8 Hz, 1H), 7.43 (dd, J = 8.2, 1.8 Hz, 1H), 7.37 (d, J = 7.9 Hz, 1H), 3.87 (q, J = 7.1Hz, 2H), 3.34-3.20 (m, 3H), 2.77 (s, 3H), 0.89 (t, J = 7.1 Hz, 3H). | 267.1 [M + H]⁺. |

### 39A Ethyl 5-(4-bromo-2-fluorophenyl)-1-(oxan-4-yl)pyrazole-4-carboxylate

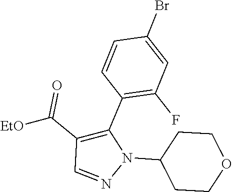 [SMILES: CCOC(=O)c1cnn(C2CCOCC2)c1-c1ccc(Br)cc1F]

Tetrahydro-2H-pyran-4-ylhydrazine hydrochloride (377 mg, 2.47 mmol) and NEt₃ (0.34 mL, 2.47 mmol) were added to a cooled (0° C.) suspension of intermediate 38A (850 mg, 2.47 mmol) in EtOH (25 mL). The mixture was warmed to rt over 10 min, stirred at rt for 16 h, then heated at 40° C. for 3 h. The reaction was concentrated under reduced pressure, and purified by flash chromatography (10-50% EtOAc/heptane) to afford a colourless oil (833 mg, 84%). ¹H NMR (700 MHz, DMSO-d₆) δ 8.05 (s, 1H), 7.77 (dd, J=9.3, 1.9 Hz, 1H), 7.59 (dd, J=8.2, 1.9 Hz, 1H), 7.46 (t, J=8.0 Hz, 1H), 4.14-3.99 (m, 3H), 3.92-3.83 (m, 2H), 3.36-3.29 (m, 2H), 2.14-2.02 (m, 2H), 1.85-1.76 (m, 1H), 1.68-1.58 (m, 1H), 1.06 (t, J=7.1 Hz, 3H). LRMS m/z: 397.6/399.7 [M+H]⁺.

### 39B Ethyl 5-(6-bromo-2-fluoropyridin-3-yl)-1-(oxan-4-yl)pyrazole-4-carboxylate

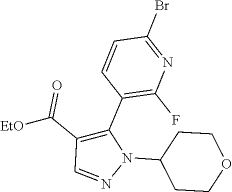 [SMILES: CCOC(=O)c1cnn(C2CCOCC2)c1-c1ccc(Br)nc1F]

NEt₃ (0.25 mL, 1.8 mmol) was added to a suspension of tetrahydro-2H-pyran-4-ylhydrazine hydrochloride (274 mg, 1.80 mmol) in CH₂Cl₂ (20 mL) and the reaction was stirred at rt for 2 h. The reaction was concentrated under reduced pressure and placed under N₂. Intermediate 38B (620 mg, 1.8 mmol) in EtOH (20 mL) was added and the mixture was stirred at rt for 18 h, then heated at 40° C. for 3 h. The mixture was concentrated under reduced pressure and purified by flash chromatography (10-50% EtOAc/heptane) to afford an off white solid (340 mg, 48%). ¹H NMR (400 MHz, DMSO-d₆) δ 8.13-8.04 (m, 2H), 7.84 (dd, J=7.8, 1.1 Hz, 1H), 4.31-4.17 (m, 1H), 4.14-4.01 (m, 2H), 3.94-3.79 (m, 2H), 3.40-3.34 (m, 2H), 2.15-1.99 (m, 2H), 1.84 (d, J=12.7 Hz, 1H), 1.66 (d, J=12.7 Hz, 1H), 1.07 (t, J=7.1 Hz, 3H). LRMS m/z: 398.1/400.0 [M+H]⁺.

The following intermediate compounds were prepared by an analogous procedure to that described for intermediate 39B.

| Preparatory | | | | LRMS |
|---|---|---|---|---|
| Example | Structure | Name | ¹H NMR (DMSO-d₆) δ | m/z |
| 39C | 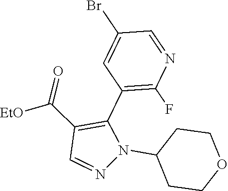 [SMILES: CCOC(=O)c1cnn(C2CCOCC2)c1-c1cc(Br)cnc1F] | Ethyl 5-(5- bromo-2- fluoropyridin- 3-yl)-1-(oxan- 4-yl)pyrazole- 4-carboxylate | (400 MHz,) δ 8.61 (d, J = 2.4 Hz, 1H), 8.47 (dd, J = 8.2, 2.5 Hz, 1H), 8.09 (s, 1H), 4.32- 4.18 (m, 1H), 4.06 (q, J = 7.0 Hz, 2H), 3.98-3.73 (m, 2H), 3.41-3.33 (m, 2H), 2.18- 1.93 (m, 2H), 1.93-1.74 (m, 1H), 1.74-1.55 (m, 1H), 1.05 (t, J = 7.1 Hz, 3H) | 398.2/ 400.2 [M + H]⁺ |
| 39D | 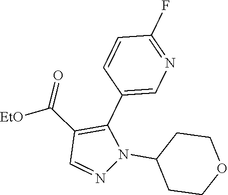 [SMILES: CCOC(=O)c1cnn(C2CCOCC2)c1-c1ccc(F)nc1] | Ethyl 5-(2,6- difluoropyridin- 3-yl)-1- (oxan-4- yl)pyrazole-4- carboxylate | (600 MHz) δ 8.35-8.32 (m, 1H), 8.12 (td, J = 8.1, 2.5 Hz, 1H), 8.05 (s, 1H), 7.42-7.34 (m, 1H), 4.23-4.10 (m, 1H), 4.05 (q, J = 7.1 Hz, 2H), 3.91- 3.82 (m, 2H), 3.31-3.27 (m, 1H), 2.54-2.51 (m, 1H), 2.14- 2.02 (m, 2H), 1.82-1.72 (m, 2H), 1.06 (t, J = 7.1 Hz, 3H). | 320.1 [M + H]⁺ |

### 39E Ethyl 5-(2,6-difluoropyridin-3-yl)-1-(1-methylpiperidin-4-yl)pyrazole-4-carboxylate

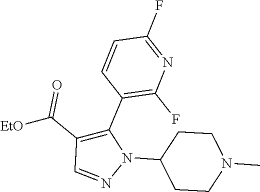 [SMILES: CCOC(=O)c1cnn(C2CCN(C)CC2)c1-c1ccc(F)nc1F]

A solution of 4-hydrazino-1-methylpiperidine (450 mg, 3.48 mmol) and intermediate 38D (990 mg, 3.48 mmol) in EtOH (30 mL) was stirred at rt for 44 h. The reaction was concentrated under reduced pressure and purified by flash chromatography [0-60% (EtOH:CH₂Cl₂:NH₄OH; 50:8:1) in CH₂Cl₂] to afford a yellow oil (480 mg, 39%). ¹H NMR (400 MHz, DMSO-d₆) δ 8.33 (q, J=8.2 Hz, 1H), 8.07 (s, 1H), 7.39 (dd, J=8.1, 2.4 Hz, 1H), 4.16-4.01 (m, 2H), 3.96-3.78 (m, 1H), 2.90-2.70 (m, 2H), 2.13 (s, 3H), 2.12-1.95 (m, 2H), 1.94-1.78 (m, 3H), 1.73-1.60 (m, 1H), 1.05 (t, J=7.1 Hz, 3H). LRMS m/z: 351.3 [M+H]⁺.

### 39F Ethyl 5-(4-bromo-2-fluorophenyl)-1-(1-methylpiperidin-4-yl)pyrazole-4-carboxylate

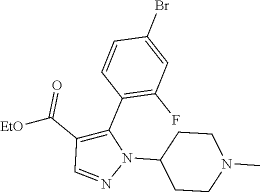 [SMILES: CCOC(=O)c1cnn(C2CCN(C)CC2)c1-c1ccc(Br)cc1F]

Prepared by an analogous procedure to intermediate 39E from intermediate 38A and 4-hydrazino-1-methylpiperidine dihydrochloride. ¹H NMR (400 MHz, DMSO-d₆) δ 8.08 (s, 1H), 7.80 (dd, J=9.3, 1.9 Hz, 1H), 7.61 (dd, J=8.2, 1.9 Hz, 1H), 7.48 (t, J=8.0 Hz, 1H), 4.16-3.94 (m, 3H), 3.25-3.08 (m, 3H), 2.32-2.15 (m, 2H), 2.08-1.88 (m, 1H), 1.88-1.73 (m, 1H), 1.07 (t, J=7.1 Hz, 3H). LRMS m/z: 410.4/412.4 [M+H]⁺.

### 40A Ethyl 5-(5-cyclopropyl-2-fluoropyridin-3-yl)-1-ethylpyrazole-4-carboxylate

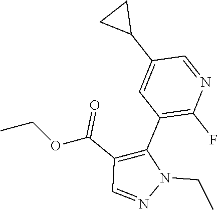 [SMILES: CCOC(=O)c1cnn(CC)c1-c1cc(C2CC2)cnc1F]

A reaction vessel was charged with Pd-170 (20 mg, 0.030 mmol), K₂CO₃ (209 mg, 1.51 mmol) and potassium cyclopropyltrifluoroborate (90 mg, 0.61 mmol). The vessel was evacuated, purged with N₂ and then a solution of intermediate 37A (150 mg, 0.50 mmol) in THF:water (1:1, 2 mL) was added. The reaction mixture was sparged with N₂, then heated to 70° C. overnight. Water (50 mL) and EtOAc (50 mL) were added and the separated aqueous phase extracted with EtOAc (2×20 mL). The combined organic phases were washed with brine (100 mL), dried (MgSO₄), and concentrated under reduced pressure. Purification by flash chromatography (0-10% MTBE/isohexanes) afforded a pale-yellow oil (98 mg, 61%). ¹H NMR (500 MHz, CDCl₃) δ 8.14-8.10 (m, 1H), 8.04 (s, 1H), 7.42 (dd, J=8.6, 2.6 Hz, 1H), 4.15 (q, J=7.1 Hz, 2H), 4.02-3.95 (m, 2H), 2.01-1.92 (m, 1H), 1.39 (t, J=7.2 Hz, 3H), 1.18-1.15 (m, 3H), 1.10-1.06 (m, 2H), 0.75-0.72 (m, 2H). LCMS (method A) 304.3 [M+H]⁺ at 1.37 min.

### 40B Ethyl 5-(5-cyclopropyl-2-fluoropyridin-3-yl)-1-(oxan-4-yl)pyrazole-4-carboxylate

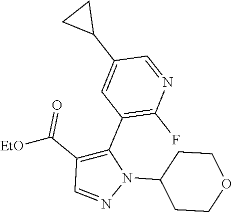 [SMILES: CCOC(=O)c1cnn(C2CCOCC2)c1-c1cc(C2CC2)cnc1F]

A solution of intermediate 39C (300 mg, 0.75 mmol) in toluene (3 mL) and water (0.3 mL) was degassed with N₂ for 15 min. K₂CO₃ (312 mg, 2.26 mmol) and potassium cyclopropyltrifluoroborate (167 mg, 1.13 mmol) were added, followed by RuPhos (35.1 mg, 0.08 mmol) and Pd(OAc)₂ (10.2 mg, 0.05 mmol). The vial was sealed and heated at 100° C. for 2 h, then at 80° C. overnight. The mixture was sparged with N₂ for 15 min, additional Pd(OAc)₂ (5.1 mg, 0.02 mmol) and RuPhos (17.6 mg, 0.04 mmol) added, and the reaction heated at 100° C. for 4 h. After cooling to rt, the reaction was diluted with CH₂Cl₂ (20 mL) and filtered through a pad of Celite, washing with CH₂Cl₂ (2×15 mL). The filtrate was dried (Na₂SO₄) and concentrated under reduced pressure. Purification by flash chromatography (10-50% EtOAc/heptane) afforded a colourless oil (85 mg, 31%). LRMS m/z: 360.3 [M+H]⁺. TLC R_{f}=0.23 (2:1 EtOAc/heptane).

### 40C Ethyl 5-(2-fluoro-4-prop-1-en-2-ylphenyl)-1-(oxan-4-yl)pyrazole-4-carboxylate

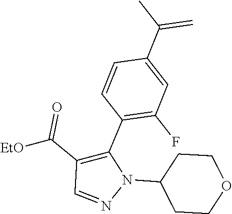 [SMILES: C=C(C)c1ccc(-c2c(C(=O)OCC)cnn2C2CCOCC2)c(F)c1]

A solution of intermediate 39A (120 mg, 0.3 mmol) in toluene (1.5 mL) and water (0.1 mL) was degassed with N₂ for 15 min. Potassium trifluoro(prop-1-en-2-yl)borate (67 mg, 0.45 mmol) was added followed by K₂CO₃ (125 mg, 0.91 mmol), RuPhos (14.1 mg, 0.03 mmol) and Pd(OAc)₂ (4.1 mg, 0.02 mmol), and the reaction heated at 100° C. for 6 h. Upon cooling to rt, the mixture was diluted with CH₂Cl₂ (10 mL) and filtered through a pad of Celite, washing with CH₂Cl₂ (3×10 mL). The filtrate was concentrated under reduced pressure and purified by flash chromatography (10-50% EtOAc/heptane) to afford a yellow solid (92 mg, 85%). LRMS m/z: 359.2 [M+H]⁺. TLC R_{f}=0.63 (1:1 EtOAc/heptane).

The following intermediate compounds were prepared by an analogous procedure to that described for intermediate 40C.

| Preparatory | | | ¹H NMR (400 MHz, | LRMS m/z; |
|---|---|---|---|---|
| Example | Structure | Name | DMSO-d₆) δ | TLC R_{f} |
| 40D | 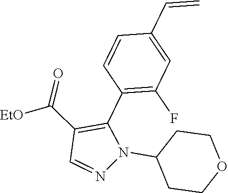 [SMILES: C=Cc1ccc(-c2c(C(=O)OCC)cnn2C2CCOCC2)c(F)c1] | Ethyl 5-(4- ethenyl-2- fluorophenyl)-1- (oxan-4- yl)pyrazole-4- carboxylate | — | 345.2 [M + H]⁺; R_{f} = 0.62 (1:1 EtOAc/ heptane) |
| 40E | 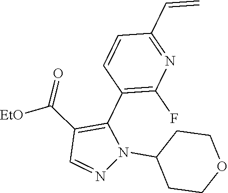 [SMILES: C=Cc1ccc(-c2c(C(=O)OCC)cnn2C2CCOCC2)c(F)n1] | Ethyl 5-(6- ethenyl-2- fluoropyridin-3- yl)-1-(oxan-4- yl)pyrazole-4- carboxylate | — | 346.3 [M + H]⁺; R_{f} = 0.78 (2:1 EtOAc/ heptane) |
| 40F | 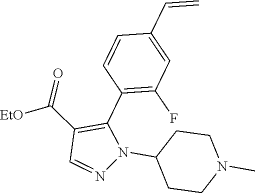 [SMILES: C=Cc1ccc(-c2c(C(=O)OCC)cnn2C2CCN(C)CC2)c(F)c1] | Ethyl 5-(4- ethenyl-2- fluorophenyl)-1- (1- methylpiperidin- 4-yl)pyrazole-4- carboxylate | — | 358.1 [M + H]⁺; R_{f} = 0.77 (100:8:1 CH₂Cl₂:EtOH: NH₄OH). |
| 40G | 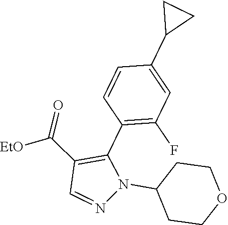 [SMILES: CCOC(=O)c1cnn(C2CCOCC2)c1-c1ccc(C2CC2)cc1F] | Ethyl 5-(4- cyclopropyl-2- fluorophenyl)-1- (oxan-4- yl)pyrazole-4- carboxylate | — | 359.0 [M + H]⁺; R_{f} = 0.79 (2:1 EtOAc/ heptane) |
| 40H | 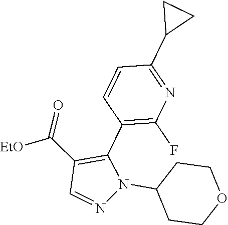 [SMILES: CCOC(=O)c1cnn(C2CCOCC2)c1-c1ccc(C2CC2)nc1F] | Ethyl 5-(6- cyclopropyl-2- fluoropyridin-3- yl)-1-(oxan-4- yl)pyrazole-4- carboxylate | 8.05 (s, 1H),7.91 (dd, J = 9.8, 7.6 Hz, 1H), 7.45 (dd, J = 7.7, 1.9 Hz, 1H), 4.20-4.07 (m, 1H), 4.11- 3.97 (m, 2H), 3.95- 3.80 (m, 2H), 2.28-2.18 (m, 1H), 2.17-1.99 (m, 2H), 1.94-1.79 (m, 1H), 1.72-1.55 (m, 1H), 1.12- 0.89 (m, 7H) | 360.2 [M + H]⁺ |

### 41A Ethyl 5-(2-fluoro-4-propan-2-ylphenyl)-1-(oxan-4-yl)pyrazole-4-carboxylate

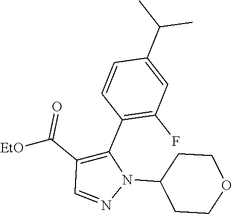 [SMILES: CCOC(=O)c1cnn(C2CCOCC2)c1-c1ccc(C(C)C)cc1F]

A solution of intermediate 40C (92 mg, 0.26 mmol) in EtOH (10 mL) in a pressure tube was evacuated and filled with N₂ (3×). Pd/C (10% wt. loading; 27.3 mg, 0.26 mmol) was added, and the reaction stirred under H₂ (20 psi) at rt for 2 h. The mixture was filtered, washing with EtOH (2×10 mL) and CH₂Cl₂ (2×10 mL) and the filtrate concentrated under reduced pressure to afford a colourless oil (70 mg, 76%). LRMS m/z: 361.1 [M+H]⁺. TLC R_{f}=0.63 (1:1 EtOAc/heptane).

The following intermediate compounds were prepared by an analogous procedure to that described for intermediate 41A.

| Preparatory | | | ¹H NMR | LRMS m/z; |
|---|---|---|---|---|
| Example | Structure | Name | (500 MHz, DMSO-d₆) δ | TLC R_{f} |
| 41B | 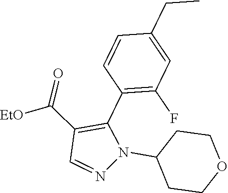 [SMILES: CCOC(=O)c1cnn(C2CCOCC2)c1-c1ccc(CC)cc1F] | Ethyl 5-(4- ethyl-2- fluorophenyl)- 1-(oxan-4- yl)pyrazole-4- carboxylate | — | 347.1 [M + H]⁺; R_{f} = 0.62 (1:1 EtOAc/ heptane). |
| 41C | 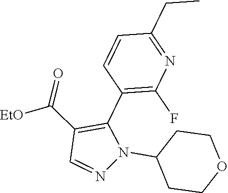 [SMILES: CCOC(=O)c1cnn(C2CCOCC2)c1-c1ccc(CC)nc1F] | Ethyl 5-(6- ethyl-2- fluoropyridin- 3-yl)-1-(oxan- 4-yl)pyrazole- 4-carboxylate | — | 348.2 [M + H]⁺; R_{f} = 0.72 (2:1 EtOAc/ heptane) |
| 41D | 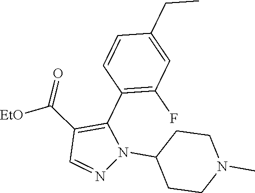 [SMILES: CCOC(=O)c1cnn(C2CCN(C)CC2)c1-c1ccc(CC)cc1F] | Ethyl 5-(4- ethyl-2- fluorophenyl)- 1-(1- methylpiperidin- 4- yl)pyrazole-4- carboxylate | 8.02 (s, 1H), 7.34 (t, J = 7.7 Hz, 1H), 7.30-7.02 (m, 2H), 4.09-3.94 (m, 2H), 3.83-3.68 (m, 1H), 2.82 (s, 2H), 2.72 (q, J = 7.6 Hz, 2H), 2.15 (s, 3H), 2.14- 1.99 (m, 2H), 1.93-1.72 (m, 3H), 1.65 (d, J = 12.6 Hz, 1H), 1.24 (t, J = 7.6 Hz, 3H), 1.04 (t, J = 7.1 Hz, 3H). | 360.2 [M + H]⁺ |

### EXAMPLES

### 1. 1-Ethyl-5-[2-fluoro-6-(methylamino)pyridin-3-yl]-N-[(3S)-2-oxo-5-phenyl-1,3-dihydro-1,4-benzodiazepin-3-yl]pyrazole-4-carboxamide

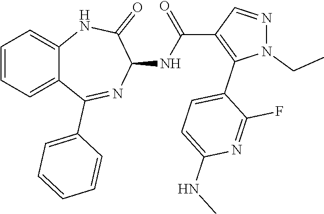 [SMILES: CCn1ncc(C(=O)N[C@H]2N=C(c3ccccc3)c3ccccc3NC2=O)c1-c1ccc(NC)nc1F]

NEt₃ (15 μL, 0.108 mmol), HATU (22 mg, 0.058 mmol) and then (S)-3-amino-5-phenyl-1H-benzo[e][1,4]diazepin-2(3H)-one (14 mg, 0.056 mmol) were added to a solution of intermediate 31E (14 mg, 0.056 mmol) in DMF (1 mL). The reaction mixture was stirred at rt overnight. Water (20 mL) was added and the precipitate was collected by filtration, washing with water. The precipitate was taken into CH₂Cl₂ (10 mL), passed through a phase separator containing brine (10 mL) and the solvent was removed under reduced pressure. Purification by flash chromatography [0-60% (10% MeOH/EtOAc)/isohexanes] afforded a white solid (21 mg, 76%). ¹H NMR (500 MHz, DMSO-d₆) δ 10.83 (s, 1H), 8.82 (d, J=8.0 Hz, 1H), 8.37 (s, 1H), 7.62 (ddd, J=8.6, 7.2, 1.6 Hz, 1H), 7.56-7.47 (m, 3H), 7.47-7.41 (m, 3H), 7.32-7.26 (m, 2H), 7.27-7.19 (m, 2H), 6.38 (dd, J=8.2, 1.8 Hz, 1H), 5.34 (d, J=7.9 Hz, 1H), 3.92 (q, J=7.2 Hz, 2H), 2.76 (d, J=4.8 Hz, 3H), 1.26 (t, J=7.2 Hz, 3H). LCMS (method B) m/z 498.2 [M+H]⁺ at 3.52 min.

The following compounds of the invention were prepared with (3S)-3-amino-5-phenyl-1,3-dihydro-1,4-benzodiazepin-2-one or (3S)-3-amino-9-fluoro-5-phenyl-1,3-dihydro-1,4-benzodiazepin-2-one by the amide coupling procedure described for the compound of Example 1.

### Compounds Prepared by Amide Coupling as in Example 1

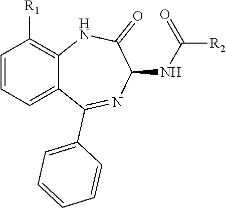

| Example | R¹ | R² | Name | ¹H NMR (DMSO-d₆) δ | LCMS/LRMS |
|---|---|---|---|---|---|
| 2 | H | 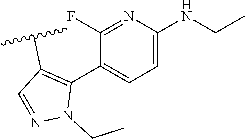 [SMILES: *c1cnn(CC)c1-c1ccc(NCC)nc1F] | 1-Ethyl-5-[6- (ethylamino)-2- fluoropyridin-3-yl]- N-[(3S)-2-oxo-5- phenyl-1,3-dihydro- 1,4-benzodiazepin- 3-yl]pyrazole-4- carboxamide | (500 MHz) δ 10.83 (s, 1H), 8.82 (d, J = 8.0 Hz, 1H), 8.37 (s, 1H), 7.62 (ddd, J = 8.6, 7.2, 1.6 Hz, 1H), 7.55- 7.48 (m, 3H), 7.48-7.43 (m, 2H), 7.46-7.39 (m, 1H), 7.32-7.27 (m, 2H), 7.27- 7.20 (m, 2H), 6.38 (dd, J = 8.3, 1.9 Hz, 1H), 5.35 (d, J = | LCMS method B 512.3 [M + H]⁺ at 3.91 min. |
| | | | | 7.9 Hz, 1H), 3.91 (q, J = 7.2 | |
| | | | | Hz, 2H), 3.27-3.18 (m, | |
| | | | | 2H), 1.26 (1, J = 7.2 Hz, 3H), | |
| | | | | 1.13 (t, J = 7.2 Hz, 3H). | |
| 3 | H | 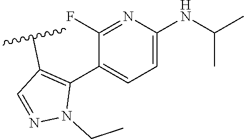 [SMILES: *c1cnn(CC)c1-c1ccc(NC(C)C)nc1F] | 1-Ethyl-5-[2-fluoro- 6-(propan-2- ylamino)pyridin-3- yl]-N-[(3S)-2-oxo- 5-phenyl-1,3- dihydro-1,4- benzodiazepin-3- yl]pyrazole-4- carboxamide | (500 MHz) δ 10.82 (s, 1H), 8.83 (d, J = 8.0 Hz, 1H), 8.37 (s, 1H), 7.62 (ddd, J = 8.6, 7.2, 1.6 Hz, 1H), 7.55- 7.48 (m, 3H), 7.48-7.42 (m, 2H), 7.45-7.37 (m, 1H), 7.32-7.26 (m, 2H), 7.26- 7.20 (m, 1H), 7.14 (d, J = 7.6 Hz, 1H), 6.37 (dd, J = | LCMS method B 526.3 [M + H]⁺ at 4.29 min. |
| | | | | 8.3, 1.9 Hz, 1H), 5.35 (d, J = | |
| | | | | 8.0 Hz, 1H), 3.95-3.87 (m, | |
| | | | | 3H), 1.26 (t, J = 7.2 Hz, 3H), | |
| | | | | 1.14 (d, J = 6.5 Hz, 6H). | |
| 4 | H | 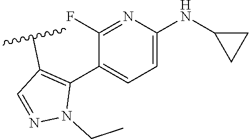 [SMILES: *c1cnn(CC)c1-c1ccc(NC2CC2)nc1F] | 1-Ethyl-5-[2-fluoro- 6- (cyclopropylamino) pyridin-3-yl]-N- [(3S)-2-oxo-5- phenyl-1,3-dihydro- 1,4-benzodiazepin- 3-yl]pyrazole-4- carboxamide | (500 MHz) δ 10.83 (s, 1H), 8.88 (d, J = 8.0 Hz, 1H), 8.40 (s, 1H), 7.62 (ddd, J = 8.6, 7.1, 1.6 Hz, 1H), 7.57- 7.41(m,7H), 7.32-7.26 (m, 2H), 7.23 (td, J = 7.5, 1.2 Hz, 1H), 6.54-6.49 (m, 1H), 5.35 (d, J = 7.9 Hz, 1H), 3.92 (q, J = 7.2 Hz, | LCMS method B 524.2 [M + H]⁺ at 3.99 min. |
| | | | | 2H), 2.55-2.51 (m, 1H), | |
| | | | | 1.26 (t, J = 7.2 Hz, 3H), | |
| | | | | 0.75-0.68 (m, 2H), 0.48-0.41 | |
| | | | | (m, 2H). | |
| 5 | H | 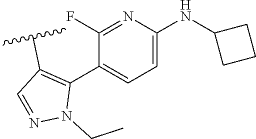 [SMILES: *c1cnn(CC)c1-c1ccc(NC2CCC2)nc1F] | 5-[6- (Cyclobutylamino)- 2-fluoropyridin-3- yl]-1-ethyl-N-[(3S)- 2-oxo-5-phenyl-1,3- dihydro-1,4- benzodiazepin-3- yl]pyrazole-4- carboxamide | (500 MHz) δ 10.82 (s, 1H), 8.85 (d, J = 8.0 Hz, 1H), 8.38 (s, 1H), 7.62 (ddd, J = 8.6, 7.1, 1.6 Hz, 1H), 7.55- 7.48 (m, 4H), 7.48-7.40 (m, 3H), 7.31-7.26 (m, 2H), 7.26-7.20 (m, 1H), 6.33 (dd, J = 8.3, 1.8 Hz, 1H), 5.34 (d, J = 7.9 Hz, 1H), | LCMS method B 538.3 [M + H]⁺ at 4.47 min. |
| | | | | 4.20-4.16 (m, 1H), 3.91 (q, | |
| | | | | J = 7.2 Hz, 2H), 2.31-2.20 | |
| | | | | (m, 2H), 1.95-1.83 (m, 2H), | |
| | | | | 1.73-1.60 (m, 2H), 1.25 (t, | |
| | | | | J = 7.2 Hz, 3H) | |
| 6 | H | 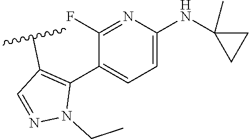 [SMILES: *c1cnn(CC)c1-c1ccc(NC2(C)CC2)nc1F] | 1-Ethyl-5-[2-fluoro- 6-[(1- methylcyclopropyl) amino]pyridin-3- yl]-N-[(3S)-2-oxo- 5-phenyl-1,3- dihydro-1,4- benzodiazepin-3- yl]pyrazole-4- | (500 MHz) δ 10.82 (s, 1H), 8.86 (d, J = 8.0 Hz, 1H), 8.39 (s, 1H), 7.62 (ddd, J = 8.6, 7.2, 1.7 Hz, 1H), 7.56- 7.48 (m, 5H), 7.48-7.41 (m, 2H), 7.32-7.26 (m, 2H), 7.26-7.20 (m, 1H), 6.48 (d, J = 8.2 Hz, 1H), 5.35 (d, J = 7.9 Hz, 1H), | LCMS method B 538.4 [M + H]⁺ at 4.35 min. |
| | | | carboxamide | 3.91 (q, J = 7.2 Hz, 2H), | |
| | | | | 1.32 (s, 3H), 1.27 (t, J = 7.2 | |
| | | | | Hz, 3H), 0.71-0.62 (m, | |
| | | | | 4H). | |
| 7 | H | 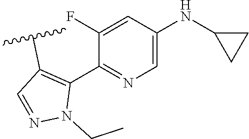 [SMILES: *c1cnn(CC)c1-c1ncc(NC2CC2)cc1F] | 5-[5- (Cyclopropylamino)- 3-fluoropyridin-2- yl]-1-ethyl-N-[(3S)- 2-oxo-5-phenyl-1,3- dihydro-1,4- benzodiazepin-3- yl]pyrazole-4- carboxamide | (500 MHz) δ 10.83 (s, 1H), 9.00 (d, J = 7.9 Hz, 1H), 8.30 (s, 1H), 7.99 (t, J = 1.9 Hz, 1H), 7.62 (ddd, J = 8.5, 7.2, 1.6 Hz, 1H), 7.55-7.48 (m, 1H), 7.51-7.46 (m, 2H), 7.48-7.41 (m, 2H), 7.32-7.27 (m, 2H), 7.27- 7.20 (m, 1H), 7.08-7.04 | LCMS method B 524.3 [M + H]⁺ at 4.04 min. |
| | | | | (m, 1H), 6.93 (dd, J = 12.2, | |
| | | | | 2.2 Hz, 1H), 5.32 (d, J = 7.8 | |
| | | | | Hz, 1H), 3.99 (q, J = 7.2 Hz, | |
| | | | | 2H), 2.45-2.39 (m, 1H), | |
| | | | | 1.26 (t, J = 7.2 Hz, 3H), | |
| | | | | 0.79-0.70 (m, 2H), 0.47-0.40 | |
| | | | | (m, 2H). | |
| 8 | H | 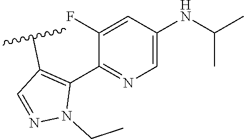 [SMILES: *c1cnn(CC)c1-c1ncc(NC(C)C)cc1F] | 1-Ethyl-5-[3-fluoro- 5-(propan-2- ylamino)pyridin-2- yl]-N-[(3S)-2-oxo- 5-phenyl-1,3- dihydro-1,4- benzodiazepin-3- yl]pyrazole-4- carboxamide | (500 MHz) δ 10.83 (s, 1H), 8.97 (d, J = 7.8 Hz, 1H), 8.26 (s, 1H), 7.94-7.90 (m, 1H), 7.62 (ddd, J = 8.5, 7.1, 1.6 Hz, 1H), 7.55-7.41 (m, 5H), 7.32-7.26 (m, 2H), 7.29-7.20 (m, 1H), 6.82 (dd, J = 12.7, 2.3 Hz, 1H), 6.55-6.49 (m, 1H), 5.32 (d, | LCMS method B 526.3 [M + H]⁺ at 4.19 min. |
| | | | | J = 7.9 Hz, 1H), 3.99 (q, J = | |
| | | | | 7.2 Hz, 2H), 3.66-3.55 (m, | |
| | | | | 1H), 1.26 (t, J = 7.2 Hz, 3H), | |
| | | | | 1.15 (d, J = 6.3 Hz, 6H). | |
| 9 | H | 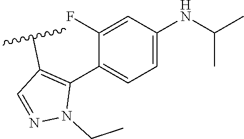 [SMILES: *c1cnn(CC)c1-c1ccc(NC(C)C)cc1F] | 1-Ethyl-5-[2-fluoro- 4-(propan-2- ylamino)phenyl]-N- [(3S)-2-oxo-5- phenyl-1,3-dihydro- 1,4-benzodiazepin- 3-yl]pyrazole-4- carboxamide | (500 MHz) δ 10.82 (s, 1H), 8.45 (d, J = 7.9 Hz, 1H), 8.27 (s, 1H), 7.62 (ddd, J = 8.6, 7.2, 1.6 Hz, 1H),7.55- 7.48 (m, 1H), 7.51-7.45 (m, 2H), 7.48-7.41 (m, 2H), 7.32-7.26 (m, 2H), 7.26-7.20 (m, 1H), 7.03 (t, | LCMS method B 525.4 [M + H]⁺ at 4.60 min. |
| | | | | J = 8.6 Hz, 1H), 6.45 (dd, | |
| | | | | J = 8.5, 2.2 Hz, 1H), 6.42- | |
| | | | | 6.36 (m, 1H), 6.14 (d, J = | |
| | | | | 7.8 Hz, 1H), 5.31 (d, J = 7.8 | |
| | | | | Hz, 1H), 3.92-3.87 (m, | |
| | | | | 2H), 3.60-3.50 (m, 1H), | |
| | | | | 1.24 (t, J = 7.2 Hz, 3H), 1.13 | |
| | | | | (d, J = 6.3 Hz, 6H). | |
| 10 | H | 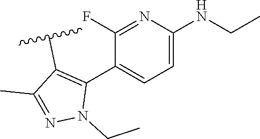 [SMILES: *c1c(C)nn(CC)c1-c1ccc(NCC)nc1F] | 1-Ethyl-5-[6- (ethylamino)-2- fluoropyridin-3-yl]- 3-methyl-N-[(3S)-2- oxo-5-phenyl-1,3- dihydro-1,4- benzodiazepin-3- yl]pyrazole-4- carboxamide | (500 MHz) δ 10.87 (s, 1H), 7.67-7.60 (m, 2H), 7.58 (t, J = 9.1 Hz, 1H), 7.55-7.49 (m, 1H), 7.48-7.42 (m, 4H), 7.40 (s, 1H), 7.34-7.28 (m, 2H), 7.27-7.23 (m, 1H), 6.49 (dd, J = 8.4, 1.9 Hz, 1H), 5.22 (d, J = 7.5 Hz, 1H), 3.86 (q, J = 7.2 Hz, | LCMS method B 526.2 [M + H]⁺ at 4.03 min. |
| | | | | 2H), 3.32-3.21 (m, 2H), | |
| | | | | 2.40 (s, 3H), 1.25 (t, J = 7.2 | |
| | | | | Hz, 3H), 1.16 (t, J = 7.2 Hz, | |
| | | | | 3H). | |
| 11 | F | 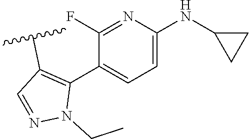 [SMILES: *c1cnn(CC)c1-c1ccc(NC2CC2)nc1F] | 5-[6- (Cyclopropylamino)- 2-fluoropyridin-3- yl]-1-ethyl-N-[(3S)- 9-fluoro-2-oxo-5- phenyl-1,3-dihydro- 1,4-benzodiazepin- 3-yl]pyrazole-4- carboxamide | (500 MHz) δ 10.80 (s, 1H), 8.92 (d, J = 7.9 Hz, 1H), 8.40 (s, 1H), 7.61-7.56 (m, 1H), 7.56-7.51 (m, 4H), 7.50-7.43 (m, 3H), 7.29 (td, J = 8.1, 5.0 Hz, 1H), 7.13 (d, J = 7.9 Hz, 1H), 6.52 (d, J = 8.4 Hz, 1H), 5.41 (d, J = 7.8 Hz, 1H), | LCMS method B 542.3 [M + H]⁺ at 4.04 min. |
| | | | | 3.92 (q, J = 7.2 Hz, 2H), | |
| | | | | 1.26 (t, J = 7.2 Hz, 3H), | |
| | | | | 0.75-0.68 (m, 2H), 0.48-0.41 | |
| | | | | (m, 2H). | |
| 12 | F | 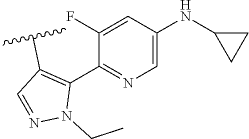 [SMILES: *c1cnn(CC)c1-c1ncc(NC2CC2)cc1F] | 5-[5- (Cyclopropylamino)- 3-fluoropyridin-2- yl]-1-ethyl-N-[(3S)- 9-fluoro-2-oxo-5- phenyl-1,3-dihydro- 1,4-benzodiazepin- 3-yl]pyrazole-4- carboxamide | (500 MHz) δ 10.80 (s, 1H), 9.04 (d, J = 7.8 Hz, 1H), 8.31 (s, 1H), 7.99 (t, J = 1.9 Hz, 1H), 7.61-7.54 (m, 1H), 7.57-7.50 (m, 3H), 7.49-7.43 (m, 2H), 7.29 (td, J = 8.1, 5.0 Hz, 1H), 7.14 (d, J = 8.1 Hz, 1H), 7.09-7.04 (m, 1H), 6.93 | LCMS method B 542.3 [M + H]⁺ at 4.06 min. |
| | | | | (dd, J = 12.2, 2.2 Hz, 1H), | |
| | | | | 5.39 (d, J = 7.7 Hz, 1H), | |
| | | | | 3.99 (q, J = 7.2 Hz, 2H), | |
| | | | | 2.44-2.39 (m, 1H), 1.26 (t, | |
| | | | | J = 7.2 Hz, 3H), 0.83-0.72 | |
| | | | | (m, 2H), 0.47-0.40 (m, | |
| | | | | 2H). | |
| 13 | F | 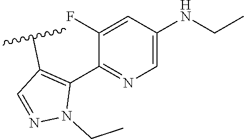 [SMILES: *c1cnn(CC)c1-c1ncc(NCC)cc1F] | 1-Ethyl-5-[5- (ethylamino)-3- fluoropyridin-2-yl]- N-[(3S)-9-fluoro-2- oxo-5-phenyl-1,3- dihydro-1,4- benzodiazepin-3- yl]pyrazole-4- carboxamide | (500 MHz, DMSO-d) δ 10.80 (s, 1H), 9.01 (d, J = 7.8 Hz, 1H), 8.27 (s, 1H), 7.95-7.91 (m, 1H), 7.61- 7.54 (m, 1H), 7.54-7.50 (m, 3H), 7.49-7.43 (m, 2H), 7.33-7.25 (m, 1H), 7.16-7.11 (m, 1H), 6.81 (dd, J = 12.6, 2.3 Hz, 1H), | LCMS method B 530.4 [M + H]⁺ at 3.93 min |
| | | | | 6.65-6.59 (m, 1H), 5.39 (d, | |
| | | | | J = 7.7 Hz, 1H), 3.99 (q, J = | |
| | | | | 7.2 Hz, 2H), 3.15-3.06 (m, | |
| | | | | 2H), 1.25 (t, J = 7.2 Hz, 3H), | |
| | | | | 1.18 (t, J = 7.1 Hz, 3H). | |
| 14 | H | 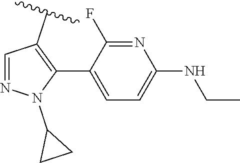 [SMILES: *c1cnn(C2CC2)c1-c1ccc(NCC)nc1F] | 1-Cyclopropyl-5-[6- (ethylamino)-2- fluoropyridin-3-yl]- N-[(3S)-2-oxo-5- phenyl-1,3-dihydro- 1,4-benzodiazepin- 3-yl]pyrazole-4- carboxamide | (400 MHz) δ 10.84 (s, 1H), 8.87 (d, J = 8.0 Hz, 1H), 8.30 (s, 1H), 7.62 (ddd, J = 8.6, 7.1, 1.7 Hz, 1H), 7.58- 7.41 (m, 6H), 7.34-7.17 (m, 4H), 6.38 (dd, J = 8.3, 2.0 Hz, 1H), 5.34 (d, J = 7.9 Hz, 1H), 3.43 (dt, J = 7.3, 3.6 Hz, 1H), 3.31-3.14 (m, 2H), 1.13 (t, J = 7.2 Hz, 3H), 1.02-0.95 (m, 2H).0.98 | LRMS 524.2 [M + H]⁺ |
| | | | | (dd, J = 4.3, 2 0.92-0.77 | |
| | | | | (m, 2H). | |
| 15 | H | 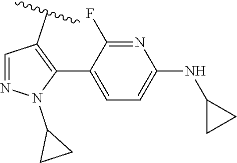 [SMILES: *c1cnn(C2CC2)c1-c1ccc(NC2CC2)nc1F] | 1-Cyclopropyl-5-[6- (cyclopropylamino)- 2-fluoropyridin-3- yl]-N-[(3S)-2-oxo- 5-phenyl-1,3- dihydro-1,4- benzodiazepin-3- yl]pyrazole-4- carboxamide | (400 MHz) δ 10.84 (s, 1H), 8.93 (d, J = 7.9 Hz, 1H), 8.32 (s, 1H), 7.69-7.56 (m, 2H), 7.56-7.42 (m, 6H), 7.37-7.19 (m, 3H), 6.52 (d, J = 8.2 Hz, 1H), 5.35 (d, J = 7.9 Hz, 1H), 3.45 (tt, J = 7.4, 3.8 Hz, 1H), 1.09-0.97 (m, 2H), 0.93-0.82 (m, 2H), 0.76-0.69 (m, 2H), 0.52- | LRMS 536.6 [M + H]⁺ |
| | | | | 0.31 (m, 2H). | |
| 16 | H | 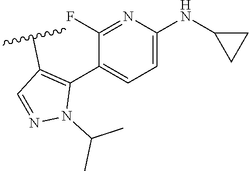 [SMILES: *c1cnn(C(C)C)c1-c1ccc(NC2CC2)nc1F] | 5-[6- (Cyclopropylamino)- 2-fluoropyridin-3- yl]-N-[(3S)-2-oxo- 5-phenyl-1,3- dihydro-1,4- benzodiazepin-3- yl]-1-propan-2- ylpyrazole-4- carboxamide | (500 MHz) δ 10.82 (s, 1H), 8.86-8.83 (m, 1H), 8.41 (s, 1H), 7.66-7.59 (m, 1H), 7.55-7.41 (m, 7H), 7.32- 7.26 (m, 2H), 7.26-7.20 (m, 1H), 6.52 (s, 1H), 5.34 (d, J = 7.9 Hz, 1H), 4.26- 4.17 (m, 1H), 2.55-2.51 (m, 1H), 1.34 (d, J = 6.4 Hz, 6H), 0.74-0.69 (m, 2H), 0.48-0.41 (m, 2H). | LCMS method D; 538.3 [M + H]⁺ at 4.36 min. |
| 17 | F | 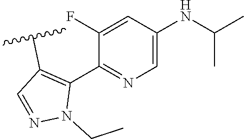 [SMILES: *c1cnn(CC)c1-c1ncc(NC(C)C)cc1F] | 1-Ethyl-N-[(3S)-9- fluoro-2-oxo-5- phenyl-1,3-dihydro- 1,4-benzodiazepin- 3-yl]-5-[3-fluoro-5- (propan-2- ylamino)pyridin-2- yl]pyrazole-4- carboxamide | (500 MHz) δ 10.80 (s, 1H), 9.02 (d, J = 7.8 Hz, 1H), 8.27 (s, 1H), 7.94-7.90 (m, 1H), 7.61-7.54 (m, 1H), 7.57-7.50 (m, 2H), 7.53- 7.49 (m, 1H), 7.49-7.43 (m, 2H), 7.29 (td, J = 8.1, 5.0 Hz, 1H), 7.16-7.11 (m, 1H), 6.82 (dd, J = 12.7, 2.3 | LCMS method B 544.3 [M + H]⁺ at 4.22 min. |
| | | | | Hz, 1H), 6.55-6.50 (m, | |
| | | | | 1H), 5.39 (d, J = 7.7 Hz, | |
| | | | | 1H), 3.99 (q, J = 7.2 Hz, | |
| | | | | 2H), 3.61 (dq, J = 13.2, 6.4 | |
| | | | | Hz, 1H), 1.26 (t, J = 7.2 Hz, | |
| | | | | 3H), 1.16 (d, J = 6.4 Hz, | |
| | | | | 6H). | |
| 18 | H | 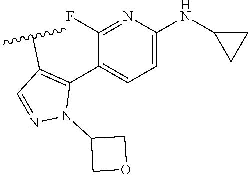 [SMILES: *c1cnn(C2COC2)c1-c1ccc(NC2CC2)nc1F] | 5-[6- (Cyclopropylamino)- 2-fluoropyridin-3- yl]-1-(oxetan-3-yl)- N-[(3S)-2-oxo-5- phenyl-1,3-dihydro- 1,4-benzodiazepin- 3-yl]pyrazole-4- carboxamide | (500 MHz): 10.83 (s, 1H), 9.03 (d, J = 8.0 Hz, 1H), 8.57 (s, 1H), 7.67-7.59 (m, 1H), 7.56-7.42 (m, 7H), 7.32-7.27 (m, 2H), 7.27- 7.20 (m, 1H), 6.52-6.47 (m, 1H), 5.35 (d, J = 7.9 Hz, 1H), 5.33-5.25 (m, 1H), 5.05-4.67 (m, 4H), 0.76- 0.68 (m, 2H), 0.48-0.41 (m, 2H). | LCMS method B 552 [M + H]⁺ at 3.73 min. |
| 19 | F | 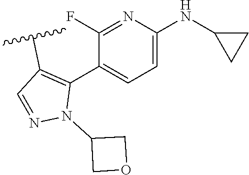 [SMILES: *c1cnn(C2COC2)c1-c1ccc(NC2CC2)nc1F] | 5-[6- (Cyclopropylamino)- 2-fluoropyridin-3- yl]-1-(oxetan-3-yl)- N-[(3S)-9-fluoro-2- oxo-5-phenyl-1,3- dihydro-1,4- benzodiazepin-3- yl]pyrazole-4- carboxamide | (400 MHz) δ 10.82 (s, 1H), 9.12 (d, J = 7.8 Hz, 1H), 8.59 (s, 1H), 7.65-7.43 (m, 8H), 7.29 (td, J = 8.1, 5.1 Hz, 1H), 7.13 (d, J = 7.9 Hz, 1H), 6.50 (d, J = 8.3 Hz, 1H), 5.42 (d, J = 7.8 Hz, 1H), 5.29 (q, J = 7.0 Hz, 1H), 5.05-4.85 (m, 4H), 0.72 (td, J = 6.7, 4.6 Hz, 2H), 0.55-0.38 (m, 2H). | LRMS 570.2 [M + H]⁺ |
| 20 | H | 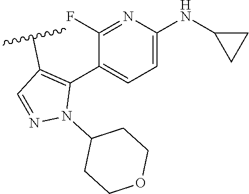 [SMILES: *c1cnn(C2CCOCC2)c1-c1ccc(NC2CC2)nc1F] | 5-[6- (Cyclopropylamino)- 2-fluoropyridin-3- yl]-N-[(3S)-2-oxo- 5-phenyl-1,3- dihydro-1,4- benzodiazepin-3- yl]-1-(oxan-4- yl)pyrazole-4- carboxamide | (500 MHz) δ 10.82 (s, 1H), 8.93-8.85 (m, 1H), 8.43 (s, 1H), 7.66-7.58 (m, 1H), 7.56-7.44 (m, 7H), 7.31- 7.26 (m, 2H), 7.25-7.21 (m, 1H), 6.56-6.48 (m, 1H), 5.34 (d, J = 7.9 Hz, 1H), 4.14-4.05 (m, 1H), 3.95-3.87 (m, 2H), 3.40- 3.33 (m, 2H), 2.11 (d, J = 16.3 Hz, 2H), 1.74 (d, J = 17.3 Hz, 2H), 0.76-0.69 | LCMS method D; 580.4 [M + H]⁺ at 3.94 min |
| | | | | (m, 2H), 0.50-0.42 (m, | |
| | | | | 2H). | |
| 21 | F | 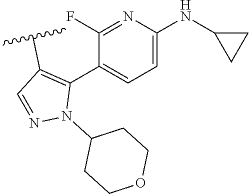 [SMILES: *c1cnn(C2CCOCC2)c1-c1ccc(NC2CC2)nc1F] | 5-[6- (Cyclopropylamino)- 2-fluoropyridin-3- yl]-N-[(3S)-9- fluoro-2-oxo-5- phenyl-1,3-dihydro- 1,4-benzodiazepin- 3-yl]-1-(oxan-4- yl)pyrazole-4- carboxamide | (400 MHz) δ 10.81 (s, 1H), 8.97 (d, J = 10.4 Hz, 1H), 8.45 (s, 1H), 7.67-7.41 (m, 8H), 7.30 (td, J = 8.1, 5.1 Hz, 1H), 7.14 (d, J = 7.9 Hz, 1H), 6.53 (s, 1H), 5.41 (d, J = 7.8 Hz, 1H), 4.20-4.02 (m, 2H), 3.92 (d, J = 11.4 Hz, 2H), 3.45-3.35 (m, 2H), 2.11 (s, 2H), 1.73 (s, 2H), 0.73 (dd, J = 6.8, 2.0 Hz, 2H), 0.53-0.42 (s, 2H). | LRMS 598.7 [M + H]⁺ |
| 22 | F | 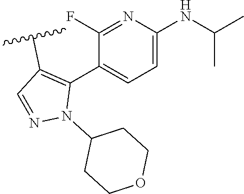 [SMILES: *c1cnn(C2CCOCC2)c1-c1ccc(NC(C)C)nc1F] | N-[(3S)-9-fluoro-2- oxo-5-phenyl-1,3- dihydro-1,4- benzodiazepin-3- yl]-5-[2-fluoro-6- (propan-2- ylamino)pyridin-3- yl]-1-(oxan-4- yl)pyrazole-4- carboxamide | (400 MHz, DMSO-d₆) δ 10.81 (s, 1H), 8.92 (t, J = 10.6 Hz, 1H), 8.43 (s, 1H), 7.63-7.37 (m, 7H), 7.29 (td, J = 8.1, 5.1Hz, 1H), 7.15 (dd, J = 15.9, 7.7 Hz, 2H), 6.43-6.30 (m, 1H), 5.41 (d, J = 7.9 Hz, 1H), 4.18-4.04 (m, 1H), 4.00- 3.82 (m, 3H), 2.19-2.00 (m, 2H), 1.81-1.61 (m, 2H), 1.14 (d, J = 6.4 Hz, 6H). | LRMS 600.3 [M + H]⁺ |
| 23 | H | 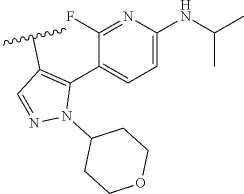 [SMILES: *c1cnn(C2CCOCC2)c1-c1ccc(NC(C)C)nc1F] | 5-[2-Fluoro-6- (propan-2- ylamino)pyridin-3- yl]-1-(oxan-4-yl)-N- [(3S)-2-oxo-5- phenyl-1,3-dihydro- 1,4-benzodiazepin- 3-yl]pyrazole-4- carboxamide | (400 MHz, DMSO-d₆) δ 10.84 (s, 1H), 8.85 (s, 1H), 8.42 (s, 1H), 7.69-7.59 (m, 1H), 7.59-7.36 (m, 6H), 7.34-7.05 (m, 4H), 6.37 (d, J = 8.3 Hz, 1H), 5.35 (d, J = 7.9 Hz, 1H), 4.21-3.98 (m, 1H), 3.98-3.81 (m, 3H), 2.21-2.01 (m, 2H), 1.82- 1.54 (m, 2H), 1.15 (d, J = 6.2 Hz, 6H). | LRMS 582.7 [M + H]⁺ |
| 24 | F | 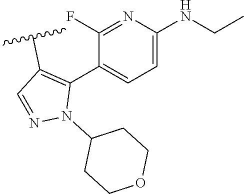 [SMILES: *c1cnn(C2CCOCC2)c1-c1ccc(NCC)nc1F] | 5-[6-(Ethylamino)- 2-fluoropyridin-3- yl]-N-[(3S)-9- fluoro-2-oxo-5- phenyl-1,3-dihydro- 1,4-benzodiazepin- 3-yl]-1-(oxan-4- yl)pyrazole-4- carboxamide | (400 MHz, DMSO-d₆) δ 10.80 (s, 1H), 8.96-8.80 (m, 1H), 8.42 (s, 1H), 7.66- 7.35 (m, 7H), 7.35-7.22 (m, 2H), 7.13 (d, J = 8.1 Hz, 1H), 6.38 (d, J = 8.2 Hz, 1H), 5.40 (d, J = 7.8 Hz, 1H), 4.17-4.03 (m, 1H), 4.03-3.79 (m, 2H), 3.27- 3.17 (m, 2H), 2.22-2.01 (m, 2H), 1.84-1.63 (m, 2H), 1.13 (t, J = 7.2 Hz, 3H). | LRMS 586.1 [M + H]⁺ |
| 25 | H | 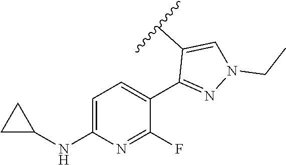 [SMILES: *c1cn(CC)nc1-c1ccc(NC2CC2)nc1F] | 3-[6- (Cyclopropylamino)- 2-fluoropyridin-3- yl]-1-ethyl-N-[(3S)- 2-oxo-5-phenyl-1,3- dihydro-1,4- benzodiazepin-3- yl]pyrazole-4- carboxamide | (400 MHz) δ 10.87 (s, 1H), 8.76 (d, J = 7.9 Hz, 1H), 8.62 (s, 1H), 7.70-7.39 (m, 7H), 7.35-7.28 (m, 2H), 7.28-7.13 (m, 2H), 6.45 (dd, J = 8.2, 1.9 Hz, 1H), 5.35 (d, J = 7.9 Hz, 1H), 4.20 (q, J = 7.3 Hz, 2H), 1.46 (t, J = 7.3 Hz, 2H), 0.70 (td, J = 6.8, 4.6 Hz, 2H), | LRMS 524.6 [M + H]⁺ |
| | | | | 0.54-0.31 (m, 2H). | |
| 26 | F | 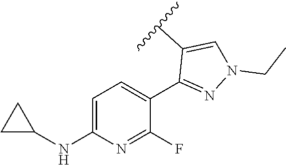 [SMILES: *c1cn(CC)nc1-c1ccc(NC2CC2)nc1F] | 3-[6- (Cyclopropylamino)- 2-fluoropyridin-3- yl]-1-ethyl-N-[(3S)- 9-fluoro-2-oxo-5- phenyl-1,3-dihydro- 1,4-benzodiazepin- 3-yl]pyrazole-4- carboxamide | (400 MHz) δ 10.84 (s, 1H), 8.81 (d, J = 7.9 Hz, 1H), 8.63 (s, 1H), 7.64-7.51 (m, 6H), 7.51-7.42 (m, 2H), 7.31 (td, J = 8.1, 5.0 Hz, 1H), 7.21 (d, J = 2.6 Hz, 1H), 7.18-7.11 (m, 1H), 6.46 (dd, J = 8.2, 1.9 Hz, 1H), 5.42 (d, J = 7.8 Hz, 1H), 4.20 (q, J = 7.3 Hz, | LRMS 542.5 [M + H]⁺ |
| | | | | 2H), 1.46 (t, J = 7.3 Hz, 3H), | |
| | | | | 0.75-0.66 (m, 2H), 0.47- | |
| | | | | 0.39 (m, 2H). | |
| 27 | H | 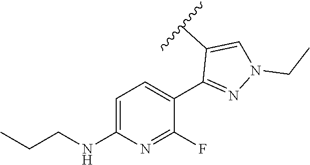 [SMILES: *c1cn(CC)nc1-c1ccc(NCCC)nc1F] | 1-Ethyl-3-[2-fluoro- 6- (propylamino)pyridin- 3-yl]-N-[(3S)-2- oxo-5-phenyl-1,3- dihydro-1,4- benzodiazepin-3- yl]pyrazole-4- carboxamide | (400 MHz) δ 10.86 (s, 1H), 8.71 (d, J = 7.9 Hz, 1H), 8.59 (s, 1H), 7.63 (ddd, J = 8.7, 7.2, 1.6 Hz, 1H),7.56- 7.41 (m, 6H), 7.35-7.20 (m, 3H), 7.00 (t, J = 5.6 Hz, 1H), 6.32 (dd, J = 8.2, 1.9 Hz, 1H), 5.35 (d, J = 7.9 Hz, 1H), 4.18 (q, J = 7.3 Hz, 2H), 3.13 (td, J = 6.8, 5.6 | LRMS 526.7 [M + H]⁺ |
| | | | | Hz, 2H), 1.60-1.48 (m, | |
| | | | | 2H), 1.45 (t, J = 7.3 Hz, 3H), | |
| | | | | 0.89 (t, J = 7.4 Hz, 3H). | |
| 28 | F | 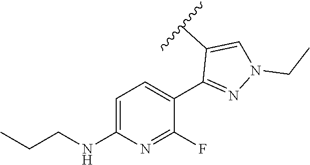 [SMILES: *c1cn(CC)nc1-c1ccc(NCCC)nc1F] | 1-Ethyl-N-[(3S)-9- fluoro-2-oxo-5- phenyl-1,3-dihydro- 1,4-benzodiazepin- 3-yl]-3-[2-fluoro-6- (propylamino)pyridin- 3-yl]pyrazole-4- carboxamide | (400 MHz) δ 10.83 (s, 1H), 8.76 (d, J = 7.9 Hz, 1H), 8.60 (s, 1H), 7.67-7.39 (m, 7H), 7.30 (td, J = 8.1, 5.0 Hz, 1H), 7.15 (d, J = 7.9 Hz, 1H), 7.00 (t, J = 5.5 Hz, 1H), 6.32 (dd, J = 8.2, 1.9 Hz, 1H), 5.41 (d, J = 7.8 Hz, 1H), 4.19 (q, J = 7.3 Hz, 2H), 3.14 (q, J = 6.4 Hz, | LRMS 544.7 [M + H]⁺ |
| | | | | 2H), 1.59-1.46 (m, 2H), | |
| | | | | 1.45 (t, J = 7.3 Hz, 3H), 0.89 | |
| | | | | (t,J =7.4 Hz, 3H). | |
| 29 | H | 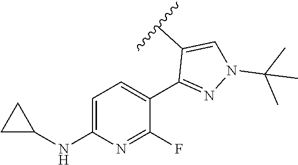 [SMILES: *c1cn(C(C)(C)C)nc1-c1ccc(NC2CC2)nc1F] | 1-tert-butyl-3-[6- (cyclopropylamino)- 2-fluoropyridin-3- yl]-N-[(3S)-2-oxo- 5-phenyl-1,3- dihydro-1,4- benzodiazepin-3- yl]pyrazole-4- carboxamide | (400 MHz) δ 10.85 (s, 1H), 8.87 (d, J = 8.0 Hz, 1H), 8.80 (s, 1H), 7.71-7.33 (m, 7H), 7.37-7.11 (m, 4H), 6.44 (dd, J = 8.2, 1.9 Hz, 1H), 5.37 (d, J = 7.9 Hz, 1H), 1.59 (s, 9H), 0.69 (td, J = 6.8, 4.7 Hz, 2H), 0.51- 0.28 (m, 2H). | LRMS 552.7 [M + H]⁺ |
| 30 | F | 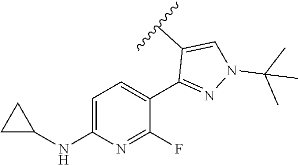 [SMILES: *c1cn(C(C)(C)C)nc1-c1ccc(NC2CC2)nc1F] | 1-tert-Butyl-3-[6- (cyclopropylamino)- 2-fluoropyridin-3- yl]-N-[(3S)-9- fluoro-2-oxo-5- phenyl-1,3-dihydro- 1,4-benzodiazepin- 3-yl]pyrazole-4- carboxamide | (400 MHz) δ 10.82 (s, 1H), 8.91 (d, J = 7.9 Hz, 1H), 8.82 (s, 1H), 7.65-7.42 (m, 7H), 7.30 (td, J = 8.1, 5.0 Hz, 1H), 7.23-7.06 (m, 2H), 6.44 (dd, J = 8.2, 1.9 Hz, 1H), 5.43 (d, J = 7.9 Hz, 1H), 1.59 (s, 9H), 0.75- 0.63 (m, 2H), 0.47-0.36 (m, 2H). | LRMS 570.7 [M + H]⁺ |
| 31 | H | 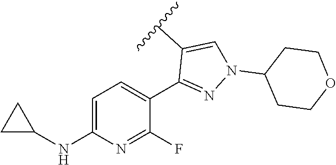 [SMILES: *c1cn(C2CCOCC2)nc1-c1ccc(NC2CC2)nc1F] | 3-[6- (Cyclopropylamino)- 2-fluoropyridin-3- yl]-1-(oxan-4-yl)-N- [(3S)-2-oxo-5- phenyl-1,3-dihydro- 1,4-benzodiazepin- 3-yl]pyrazole-4- carboxamide | (400 MHz) δ 10.86 (s, 1H), 8.80 (d, J = 7.9 Hz, 1H), 8.72 (s, 1H), 7.69-7.44 (m, 7H), 7.43-7.17 (m, 4H), 6.44 (dd, J = 8.2, 1.9 Hz, 1H), 5.35 (d, J = 7.9 Hz, 1H), 4.56-4.36 (m, 1H), 4.00 (dd, J = 11.0, 4.4 Hz, 2H), 3.56-3.41 (m, 2H), 2.20-2.07 (m, 2H), 2.07- | LRMS 580.6 [M + H]⁺ |
| | | | | 1.83 (m, 2H), 0.70 (td, J = | |
| | | | | 6.8, 4.6 Hz, 2H), 0.47-0.33 | |
| | | | | (m, 2H). | |
| 32 | F | 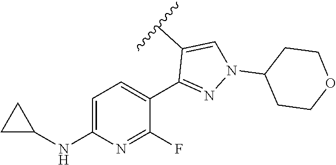 [SMILES: *c1cn(C2CCOCC2)nc1-c1ccc(NC2CC2)nc1F] | 3-[6- (Cyclopropylamino)- 2-fluoropyridin-3- yl]-N-[(3S)-9- fluoro-2-oxo-5- phenyl-2,3-dihydro- 1H-1,4- benzodiazepin-3- yl]-1-(oxan-4-yl)- 1H-pyrazole-4- | (400 MHz) δ 10.84 (s, 1H), 8.85 (d, J = 7.8 Hz, 1H), 8.73 (s, 1H), 7.65-7.42 (m, 7H), 7.38-7.10 (m, 3H), 6.45 (dd, J = 8.2, 1.9 Hz, 1H), 5.42 (d, J = 7.8 Hz, 1H), 4.54-4.39 (m, 1H), 4.00 (dd, J = 10.8, 4.3 Hz, 2H), 3.58-3.44 (m, 2H), 2.17-2.05 (m, 2H), 2.05- | LRMS 598.5 [M + H]⁺ |
| | | | carboxamide | 1.84 (m, 2H), 0.76-0.65 | |
| | | | | (m, 2H), 0.49-0.34 (m, | |
| | | | | 2H). | |
| 33 | F | 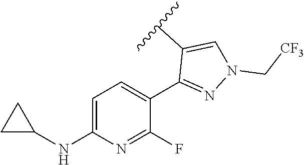 [SMILES: *c1cn(CC(F)(F)F)nc1-c1ccc(NC2CC2)nc1F] | 3-[6- (Cyclopropylamino)- 2-fluoropyridin-3- yl]-N-[(3S)-9- fluoro-2-oxo-5- phenyl-2,3-dihydro- 1H-1,4- benzodiazepin-3- yl]-1-(2,2,2- trifluoroethyl)-1H- | (400 MHz) δ 10.84 (s, 1H), 9.19 (d, J = 7.8 Hz, 1H), 8.70 (s, 1H), 7.68-7.40 (m, 7H), 7.27 (d, J = 2.8 Hz, 2H), 7.15 (d, J = 8.0 Hz, 1H), 6.46 (d, J = 8.2 Hz, 1H), 5.41 (d, J = 7.7 Hz, 1H), 5.27 (d, J = 9.0 Hz, 2H), 0.74-0.67 (m, 2H), 0.54-0.26 (m, 2H). | LRMS 596.7 [M + H]⁺ |
| | | | pyrazole-4- | | |
| | | | carboxamide | | |
| 34 | H | 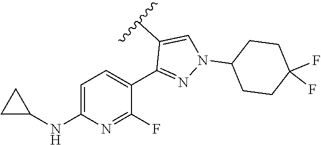 [SMILES: *c1cn(C2CCC(F)(F)CC2)nc1-c1ccc(NC2CC2)nc1F] | 3-[6- (Cyclopropylamino)- 2-fluoropyridin-3- yl]-1-(4,4- difluorocyclohexyl)- N-[(3S)-2-oxo-5- phenyl-1,3-dihydro- 1,4-benzodiazepin- 3-yl]pyrazole-4- carboxamide | (400 MHz) δ 10.86 (s, 1H), 8.82 (d, J = 7.9 Hz, 1H), 8.74 (s, 1H), 7.69-7.38 (m, 7H), 7.36-7.16 (m, 4H), 6.44 (dd, J = 8.2, 1.9 Hz, 1H), 5.35 (d, J = 7.9 Hz, 1H), 4.48 (t, J = 10.4 Hz, 1H), 2.27-1.90 (m, 8H), 0.70 (td, J = 6.8, 4.6 Hz, 2H), 0.50-0.33 (m, 2H). | LRMS 614.6 [M + H]⁺ |
| 35 | F | 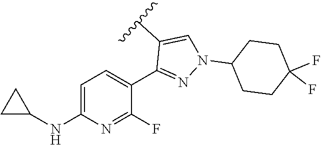 [SMILES: *c1cn(C2CCC(F)(F)CC2)nc1-c1ccc(NC2CC2)nc1F] | 3-[6- (Cyclopropylamino)- 2-fluoropyridin-3- yl]-1-(4,4- difluorocyclohexyl)- N-[(3S)-9-fluoro-2- oxo-5-phenyl-1,3- dihydro-1,4- benzodiazepin-3- yl]pyrazole-4- | (400 MHz) δ 10.84 (s, 1H), 8.87 (d, J = 7.8 Hz, 1H), 8.75 (s, 1H), 7.64-7.42 (m, 7H), 7.30 (td, J = 8.1, 5.1 Hz, 1H), 7.24-7.09 (m, 2H), 6.44 (dd, J = 8.3, 1.9 Hz, 1H), 5.42 (d, J = 7.8 Hz, 1H), 4.46 (d, J = 10.9 Hz, 1H), 2.26-1.94 (m, 8H), 0.75-0.65 (m, 2H), 0.47- | LRMS 632.7 [M + H]⁺ |
| | | | carboxamide | 0.36 (m, 2H). | |
| 36 | F | 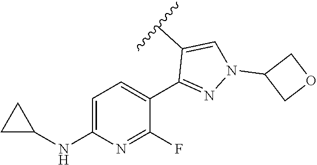 [SMILES: *c1cn(C2COC2)nc1-c1ccc(NC2CC2)nc1F] | 3-[6- (Cyclopropylamino)- 2-fluoropyridin-3- yl]-N-[(3S)-9- fluoro-2-oxo-5- phenyl-1,3-dihydro- 1,4-benzodiazepin- 3-yl]-1-(oxetan-3- yl)pyrazole-4- carboxamide | (400 MHz) δ 10.86 (s, 1H), 8.95 (d, J = 7.8 Hz, 1H), 8.83 (s, 1H), 7.70-7.54 (m, 4H), 7.54-7.41 (m, 3H), 7.41-7.23 (m, 2H), 7.16 (d, J = 7.9 Hz, 1H),6.47 (dd, J = 8.2, 1.9 Hz, 1H), 5.69- 5.54 (m, 1H), 5.43 (d, J = 7.7 Hz, 1H), 5.04-4.90 (m, 4H), 0.80-0.63 (m, 2H), | LRMS 571.0 [M + H]⁺ |
| | | | | 0.52-0.34 (m, 2H). | |
| 37 | H | 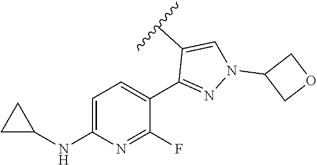 [SMILES: *c1cn(C2COC2)nc1-c1ccc(NC2CC2)nc1F] | 3-[6- (Cyclopropylamino)- 2-fluoropyridin-3- yl]-1-(oxetan-3-yl)- N-[(3S)-2-oxo-5- phenyl-1,3-dihydro- 1,4-benzodiazepin- 3-yl]pyrazole-4- carboxamide | (400 MHz) δ 10.87 (s, 1H), 8.90 (d, J = 7.9 Hz, 1H), 8.82 (s, 1H), 7.69-7.60 (m, 2H), 7.60-7.40 (m, 5H), 7.33-7.15 (m, 4H), 6.46 (dd, J = 8.2, 1.9 Hz, 1H), 5.66-5.58 (m, 1H), 5.35 (d, J = 7.9 Hz, 1H), 5.04-4.84 (m, 4H), 0.77-0.58 (m, 2H), 0.53-0.32 (m, 2H). | LRMS 553.0 [M + H]⁺ |
| 40 | F | 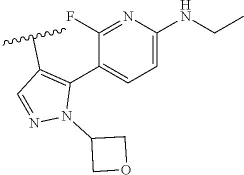 [SMILES: *c1cnn(C2COC2)c1-c1ccc(NCC)nc1F] | 5-[6-(Ethylamino)- 2-fluoropyridin-3- yl]-N-[(3S)-9- fluoro-2-oxo-5- phenyl-1,3-dihydro- 1,4-benzodiazepin- 3-yl]-1-(oxetan-3- yl)pyrazole-4- carboxamide | (700 MHz) 10.81 (s, 1H), 9.03 (d, J = 7.9 Hz, 1H), 8.56 (s, 1H), 7.60-7.55 (m, 1H), 7.55-7.52 (m, 3H), 7.47 (t, J = 7.8 Hz, 2H), 7.37 (d, J = 9.2 Hz, 1H), 7.29 (td, J = 7.8, 4.8 Hz, 2H), 7.13 (d, J = 7.9 Hz, 1H), 6.36 (d, J = 8.3 Hz, 1H), 5.42 (d, J = 7.8 Hz, 1H), 5.32-5.27 (m, 1H), 5.05-4.75 (m, 4H), | LRMS 557.7 [M + H]⁺ |
| | | | | 3.26-3.17 (m, 2H), 1.13 (t, | |
| | | | | J = 7.2 Hz, 3H). | |
| 41 | F | 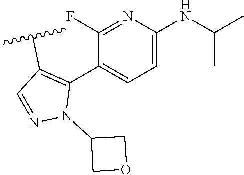 [SMILES: *c1cnn(C2COC2)c1-c1ccc(NC(C)C)nc1F] | N-[(3S)-9-Fluoro-2- oxo-5-phenyl-1,3- dihydro-1,4- benzodiazepin-3- yl]-5-[2-fluoro-6- (propan-2- ylamino)pyridin-3- yl]-1-(oxetan-3- yl)pyrazole-4- carboxamide | (700 MHz) 10.80 (s, 1H), 9.04 (d, J = 7.9 Hz, 1H), 8.56 (s, 1H), 7.63-7.51 (m, 4H), 7.51-7.43 (m, 2H), 7.36(t, J = 9.2 Hz, 1H), 7.32- 7.25 (m, 1H), 7.20 (d, J = 7.7 Hz, 1H), 7.14 (d, J = 8.0 Hz, 1H), 6.35 (d, J = 8.4 Hz, 1H), 5.42 (d, J = 7.8 Hz, 1H), 5.33-5.23 (m, 1H), 5.06-4.72 (m, 4H), 3.97- | LRMS 572.6 [M + H]⁺ |
| | | | | 3.86 (m, 1H), 1.14 (d, J = | |
| | | | | 6.5 Hz, 6H). | |
| 42 | H | 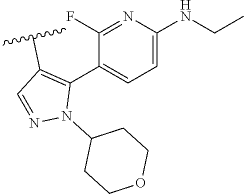 [SMILES: *c1cnn(C2CCOCC2)c1-c1ccc(NCC)nc1F] | 5-[6-(Ethylamino)- 2-fluoropyridin-3- yl]-1-(oxan-4-yl)-N- [(3S)-2-oxo-5- phenyl-1,3-dihydro- 1,4-benzodiazepin- 3-yl]pyrazole-4- carboxamide | (400 MHz) 10.83 (s, 1H), 8.87-8.80 (m, 1H), 8.41 (s, 1H), 7.72-7.55 (m, 1H), 7.55-7.34 (m, 5H), 7.34- 7.12 (m, 5H), 6.38 (d, J = 8.3 Hz, 1H), 5.33 (d, J = 7.9 Hz, 1H), 4.19-4.05 (m, 1H), 3.99-3.82 (m, 2H), 3.28-3.14 (m, 2H), 2.23- 2.00 (m, 2H), 1.85-1.62 (m, 2H), 1.13 (t, J = 7.2 Hz, 3H). | LRMS 568.2 [M + H]⁺ |
| 43 | H | 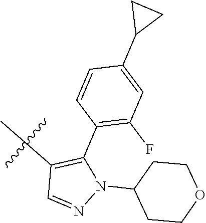 [SMILES: *c1cnn(C2CCOCC2)c1-c1ccc(C2CC2)cc1F] | 5-(4-Cyclopropyl-2- fluorophenyl)-1- (oxan-4-yl)-N- [(3S)-2-oxo-5- phenyl-1,3-dihydro- 1,4-benzodiazepin- 3-yl]pyrazole-4- carboxamide | (400 MHz) 10.83 (s, 1H), 8.92 (dd, J = 11.4, 7.9 Hz, 1H), 8.47 (s, 1H), 7.66- 7.54 (m, 1H), 7.54-7.37 (m, 5H), 7.37-7.11 (m, 4H), 7.11-6.92 (m, 2H), 5.37-5.21 (m, 1H), 4.09- 3.96 (m, 1H), 3.96-3.81 (m, 2H), 2.17-1.93 (m, 3H), 1.86-1.56 (m, 2H), 1.07-0.90 (m, 2H), 0.80- 0.69 (m, 2H). | LRMS 564.7 [M + H]⁺ |
| 44 | F | 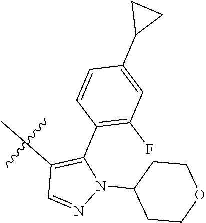 [SMILES: *c1cnn(C2CCOCC2)c1-c1ccc(C2CC2)cc1F] | 5-(4-Cyclopropyl-2- fluorophenyl)-N- [(3S)-9-fluoro-2- oxo-5-phenyl-1,3- dihydro-1,4- benzodiazepin-3- yl]-1-(oxan-4- yl)pyrazole-4- carboxamide | (400 MHz) 10.80 (s, 1H), 9.06-8.89 (m, 1H), 8.47 (s, 1H), 7.65-7.44 (m, 6H), 7.33-7.19 (m, 2H), 7.12 (d, J = 8.0 Hz, 1H), 7.05-6.95 (m, 2H), 5.38 (d, J = 7.8 Hz, 1H), 4.09-3.95 (m, 1H), 3.95-3.80 (m, 2H), 2.21- 2.04 (m, 2H), 2.04-1.93 (m, 1H), 1.81-1.72 (m, 1H), 1.70-1.61 (m, 1H), 1.11-0.95 (m, 2H), 0.85- 0.65 (m, 2H). | LRMS 581.6 [M + H]⁺ |
| 45 | F | 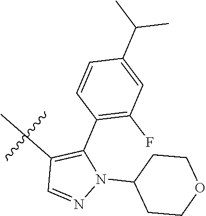 [SMILES: *c1cnn(C2CCOCC2)c1-c1ccc(C(C)C)cc1F] | 5-(2-Fluoro-4- propan-2-ylphenyl)- 1-(oxan-4-yl)-N- [(3S)-9-fluoro-2- oxo-5-phenyl-1,3- dihydro-1,4- benzodiazepin-3- yl]pyrazole-4- carboxamide | (600 MHz) δ 10.78 (s, 1H), 8.95 (dd, J = 13.6, 7.8 Hz, 1H), 8.49 (s, 1H), 7.61- 7.40 (m, 6H), 7.36-7.24 (m, 2H), 7.24-7.10 (m, 3H), 5.42-5.36 (m, 1H), 4.04-3.96 (m, 1H), 3.95- 3.81 (m, 2H), 3.31-3.23 (m, 2H), 3.01-2.92 (m, 1H), 2.14-2.06 (m, 2H), 1.81-1.74 (m, 1H), 1.71- 1.60 (m, 1H), 1.23 (d, J = 7.0 Hz, 6H). | LRMS 584.2 [M + H]⁺ |
| 46 | H | 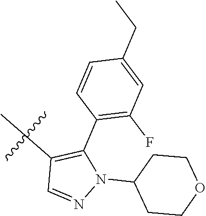 [SMILES: *c1cnn(C2CCOCC2)c1-c1ccc(CC)cc1F] | 5-(4-Ethyl-2- fluorophenyl)-1- (oxan-4-yl)-N- [(3S)-2-oxo-5- phenyl-1,3-dihydro- 1,4-benzodiazepin- 3-yl]pyrazole-4- carboxamide | (600 MHz) 10.81 (d, J = 6.2 Hz, 1H), 8.90 (dd, J = 14.9, 8.0 Hz, 1H), 8.47 (s, 1H), 7.67-7.59 (m, 1H), 7.59- 7.36 (m, 5H), 7.36-7.02 (m, 6H), 5.34-5.27 (m, 1H), 4.06-3.96 (m, 1H), 3.95-3.84 (m, 2H), 3.28 (t, J = 12.1Hz, 2H),2.67 (q, J = 7.6 Hz, 2H), 2.16-2.03 (m, 2H), 1.80-1.73 (m, 1H), 1.69-1.63 (m, 1H), 1.21 (t, J = 7.6 Hz, 3H). | LRMS 552.1 [M + H]⁺ |
| 47 | F | 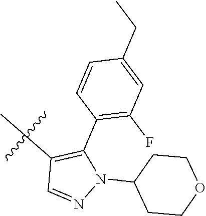 [SMILES: *c1cnn(C2CCOCC2)c1-c1ccc(CC)cc1F] | 5-(4-Ethyl-2- fluorophenyl)-1- (oxan-4-yl)-N- [(3S)-9-fluoro-2- oxo-5-phenyl-1,3- dihydro-1,4- benzodiazepin-3- yl]pyrazole-4- carboxamide | (600 MHz) 10.78 (s, 1H), 8.95 (dd, J = 14.7, 7.8 Hz, 1H), 8.48 (s, 1H), 7.61- 7.40 (m, 6H), 7.35-7.25 (m, 2H), 7.25-7.01 (m, 3H), 5.43-5.32 (m, 1H), 4.07-3.96 (m, 1H), 3.96- 3.85 (m, 2H), 3.31-3.23 (m, 2H), 2.68 (q, J = 7.6 Hz, 2H), 2.20-2.00 (m, 2H), 1.80-1.73 (m, 1H), 1.69- 1.62 (m, 1H), 1.22 (t, J = 7.6 Hz, 3H). | LRMS 570.1 [M + H]⁺ |
| 48 | F | 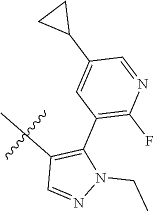 [SMILES: *c1cnn(CC)c1-c1cc(C2CC2)cnc1F] | 5-(5-Cyclopropyl-2- fluoropyridin-3-yl)- 1-ethyl-N-[(3S)-9- fluoro-2-oxo-5- phenyl-1,3-dihydro- 1,4-benzodiazepin- 3-yl]pyrazole-4- carboxamide | (500 MHz) 10.79 (s, 1H), 9.20 (d, J = 8.0 Hz, 1H), 8.52 (s, 1H), 8.12-8.08 (m, 1H), 7.66 (dd, J = 9.0, 2.5 Hz, 1H), 7.60-7.50 (m, 4H), 7.50-7.43 (m, 2H), 7.32-7.24 (m, 1H), 7.15- 7.10 (m, 1H), 5.41 (d, J = 7.9 Hz, 1H), 3.92 (q, J = 7.2 Hz, 2H), 2.05-1.96 (m, 1H), 1.24 (t, J = 7.2 Hz, 3H), 1.01-0.95 (m, 2H), 0.78- 0.73 (m, 2H). | LCMS method B 527.4 [M + H]⁺ at 4.23 min |
| 49 | F | 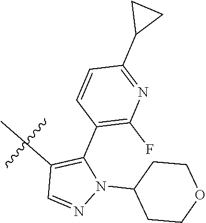 [SMILES: *c1cnn(C2CCOCC2)c1-c1ccc(C2CC2)nc1F] | 5-(6-Cyclopropyl-2- fluoropyridin-3-yl)- N-[(3S)-9-fluoro-2- oxo-5-phenyl-1,3- dihydro-1,4- benzodiazepin-3- yl]-1-(oxan-4- yl)pyrazole-4- carboxamide | (400 MHz) 10.80 (s, 1H), 9.22 (t, J = 7.8 Hz, 1H), 8.55 (s, 1H), 7.80 (t, J = 8.9 Hz, 1H), 7.67-7.43 (m, 6H), 7.43-7.19 (m, 2H), 7.12 (d, J = 7.9 Hz, 1H), 5.39 (d, J = 7.8 Hz, 1H), 4.20-3.97 (m, 1H), 3.97-3.83 (m, 2H), 2.27-2.01 (m, 3H), 1.89- 1.75 (m, 1H), 1.68-1.57 (m, 1H), 1.13-0.71 (m, 4H). | LRMS 583.3 [M + H]⁺ |
| 50 | H | 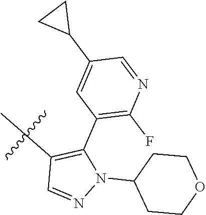 [SMILES: *c1cnn(C2CCOCC2)c1-c1cc(C2CC2)cnc1F] | 5-(5-Cyclopropyl-2- fluoropyridin-3-yl)- 1-(oxan-4-yl)-N- [(3S)-2-oxo-5- phenyl-1,3-dihydro- 1,4-benzodiazepin- 3-yl]pyrazole-4- carboxamide | (400 MHz) 10.83 (d, J = 8.5 Hz, 1H), 9.19 (dd, J = 8.4, 3.7 Hz, 1H), 8.55 (d, J = 3.0 Hz, 1H), 8.10 (s, 1H), 7.66- 7.58 (m,2H), 7.58-7.34 (m, 5H), 7.34-7.15 (m, 3H), 5.33 (dd, J = 8.1, 2.6 Hz, 1H), 4.14-3.97 (m, 1H), 3.97-3.75 (m, 2H), 2.20-1.92 (m, 3H), 1.89- 1.80 (m, 1H), 1.75-1.53 (m, 1H), 1.06-0.93 (m, 2H), 0.83-0.64 (m, 2H). | LRMS 565.4 [M + H]⁺ |
| 51 | F | 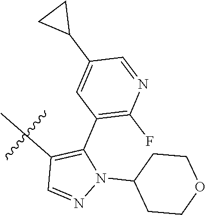 [SMILES: *c1cnn(C2CCOCC2)c1-c1cc(C2CC2)cnc1F] | 5-(5-Cyclopropyl-2- fluoropyridin-3-yl)- N-[(3S)-9-fluoro-2- oxo-5-phenyl-1,3- dihydro-1,4- benzodiazepin-3- yl]-1-(oxan-4- yl)pyrazole-4- carboxamide | (400 MHz) 10.80 (d, J = 9.3 Hz, 1H), 9.23 (dd, J = 8.1, 3.6 Hz, 1H), 8.56 (d, J = 2.4 Hz, 1H), 8.10 (d, J = 2.3 Hz, 1H), 7.71-7.34 (m, 7H), 7.27 (dd, J = 8.3, 5.0 Hz, 1H), 7.12 (d, J = 8.0 Hz, 1H), 5.40 (d, J = 7.8 Hz, 1H), 4.11-3.95 (m, 1H), 3.95-3.82 (m, 2H), 2.17- 2.00 (m, 3H), 1.91-1.78 (m, 1H), 1.78-1.59 (m, 1H), 1.11-0.92 (m, 2H), 0.83-0.64 (m, 2H). | LRMS 583.4 [M + H]⁺ |
| 52 | H | 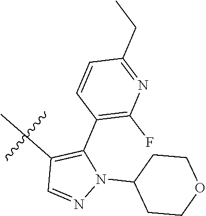 [SMILES: *c1cnn(C2CCOCC2)c1-c1ccc(CC)nc1F] | 5-(6-Ethyl-2- fluoropyridin-3-yl)- 1-(oxan-4-yl)-N- [(3S)-2-oxo-5- phenyl-1,3-dihydro- 1,4-benzodiazepin- 3-yl]pyrazole-4- carboxamide | (500 MHz) δ 10.81 (d, J = 9.3 Hz, 1H), 9.18 (d, J = 8.1 Hz, 1H), 8.55 (s, 1H), 7.88 (dd, J = 9.7, 7.5 Hz, 1H), 7.66-7.57 (m, 1H), 7.57- 7.38 (m, 5H), 7.35-7.15 (m, 4H), 5.33 (d, J = 8.0 Hz, 1H), 4.15-3.99 (m, 1H), 3.89 (d, J = 11.5 Hz, 2H), 2.92-2.68 (m, 2H), 2.20- 2.03 (m, 2H), 1.93-1.76 (m, 1H), 1.74-1.59 (m, 1H), 1.24 (t, J = 7.6 Hz, 3H). | LRMS 553.3 [M + H]⁺ |
| 53 | F | 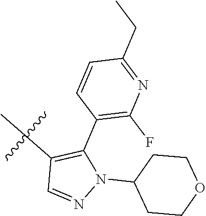 [SMILES: *c1cnn(C2CCOCC2)c1-c1ccc(CC)nc1F] | 5-(6-Ethyl-2- fluoropyridin-3-yl)- 1-(oxan-4-yl)-N- [(3S)-9-fluoro-2- oxo-5-phenyl-1,3- dihydro-1,4- benzodiazepin-3- yl]pyrazole-4- carboxamide | (500 MHz) δ 10.78 (s, 1H), 9.22 (s, 1H), 8.56 (s, 1H), 7.88 (t, J = 8.7 Hz, 1H), 7.63-7.41 (m, 6H), 7.34-7.22 (m, 2H), 7.12 (d, J = 7.9 Hz, 1H), 5.40 (d, J = 7.9 Hz, 1H), 4.13-4.00 (m, 1H), 3.96-3.83 (m, 2H), 2.83- 2.73 (m, 2H), 2.18-2.01 (m, 2H), 1.86-1.74 (m, 1H), 1.74-1.60 (m, 1H), 1.24 (t, J = 7.6 Hz, 3H). | LRMS 571.3 [M + H]⁺ |
| 54 | H | 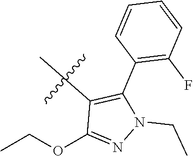 [SMILES: *c1c(OCC)nn(CC)c1-c1ccccc1F] | 3-Ethoxy-1-ethyl-5- (2-fluorophenyl)-N- [(3S)-2-oxo-5- phenyl-1,3-dihydro- 1,4-benzodiazepin- 3-yl]pyrazole-4- carboxamide | (500 MHz) 10.94 (s, 1H), 8.58-8.50 (m, 1H), 7.65- 7.58 (m, 1H), 7.56-7.47 (m, 2H), 7.43 (t, J = 3.6 Hz, 5H), 7.32-7.20 (m, 5H), 5.24 (d, J = 7.6 Hz, 1H), 4.49-4.41 (m, 2H), 3.83- 3.75 (m, 2H), 1.49 (t, J = 7.0 Hz, 3H), 1.22 (t, J = 7.1 Hz, 3H). | LCMS method C 512.4 [M + H]⁺ at 4.99 min. |
| 55 | F | 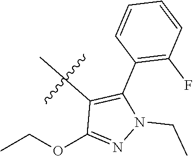 [SMILES: *c1c(OCC)nn(CC)c1-c1ccccc1F] | 3-Ethoxy-1-ethyl- N-[(3S)-9-fluoro-2- oxo-5-phenyl-1,3- dihydro-1,4- benzodiazepin-3- yl]-5-(2- fluorophenyl)pyrazole- 4-carboxamide | (500 MHz) 10.92 (s, 1H), 8.61-8.53 (m, 1H), 7.59- 7.53 (m, 1H), 7.55-7.48 (m, 2H), 7.47-7.41 (m, 5H), 7.38-7.24 (m, 3H), 7.13-7.08 (m, 1H), 5.32 (d, J = 7.5 Hz, 1H), 4.49-4.41 (m,2H), 3.83-3.75 (m, 2H), 1.49 (t, J = 7.0 Hz, 3H), 1.22 (t, J = 7.2 Hz, 3H). | LCMS method C 530.4 [M + H]⁺ at 5.08 min |

### 38. 1-Ethyl-5-(3-fluoro-5-(methylamino)pyridin-2-yl)-N—((S)-2-oxo-5-phenyl-2,3-dihydro-1H-benzo[e][1,4]diazepin-3-yl)-1H-pyrazole-4-carboxamide

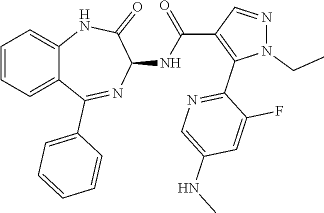 [SMILES: CCn1ncc(C(=O)N[C@H]2N=C(c3ccccc3)c3ccccc3NC2=O)c1-c1ncc(NC)cc1F]

HCl (4.0 M in dioxane; 50 μL, 0.200 mmol) was added to a solution of intermediate 34A (30 mg, 0.050 mmol) in CH₂Cl₂ (1 mL) and stirred at rt. After 1 h, further HCl (4.0 M in dioxane; 200 μL, 0.800 mmol) was added and the reaction stirred at rt over the weekend. The reaction was partitioned between CH₂Cl₂ (10 mL) and sat. aq. NaHCO₃ (10 mL), separated and the aqueous phase extracted with CH₂Cl₂ (2×10 mL). The combined organic extracts were washed with brine (20 mL), dried (MgSO₄), and the solvent removed under reduced pressure. Purification by flash chromatography [50-100% (10% MeOH/EtOAc)/isohexanes] afforded a white solid (14 mg, 56%). ¹H NMR (500 MHz, DMSO-d₆) δ 10.83 (s, 1H), 8.96 (d, J=7.8 Hz, 1H), 8.27 (s, 1H), 7.92 (t, J=1.9 Hz, 1H), 7.62 (ddd, J=8.6, 7.2, 1.6 Hz, 1H), 7.55-7.48 (m, 1H), 7.51-7.45 (m, 2H), 7.48-7.41 (m, 2H), 7.32-7.26 (m, 2H), 7.26-7.20 (m, 1H), 6.80 (dd, J=12.5, 2.3 Hz, 1H), 6.67 (s, 1H), 5.32 (d, J=7.8 Hz, 1H), 3.99 (q, J=7.2 Hz, 2H), 2.74 (d, J=5.0 Hz, 3H), 1.25 (t, J=7.2 Hz, 3H). LCMS (method B) m/z 498.2 [M+H]⁺ at 3.49 min.

### 39. 1-Ethyl-5-[5-(ethylamino)-3-fluoropyridin-2-yl]-N-[(3S)-2-oxo-5-phenyl-1,3-dihydro-1,4-benzodiazepin-3-yl]pyrazole-4-carboxamide

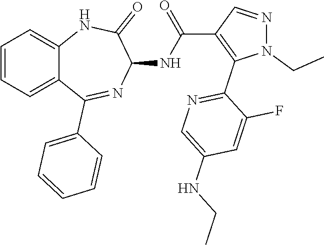 [SMILES: CCNc1cnc(-c2c(C(=O)N[C@H]3N=C(c4ccccc4)c4ccccc4NC3=O)cnn2CC)c(F)c1]

Prepared by an analogous procedure to that described for the compound of Example 38 from intermediate 34B. ¹H NMR (500 MHz, DMSO-d₆) δ 10.83 (s, 1H), 8.96 (d, J=7.8 Hz, 1H), 8.27 (s, 1H), 7.93 (t, J=1.9 Hz, 1H), 7.62 (ddd, J=8.6, 7.1, 1.6 Hz, 1H), 7.55-7.48 (m, 1H), 7.51-7.45 (m, 2H), 7.48-7.41 (m, 2H), 7.32-7.26 (m, 2H), 7.26-7.20 (m, 1H), 6.81 (dd, J=12.6, 2.3 Hz, 1H), 6.65-6.59 (m, 1H), 5.32 (d, J=7.8 Hz, 1H), 3.99 (q, J=7.2 Hz, 2H), 3.15-3.06 (m, 2H), 1.25 (t, J=7.2 Hz, 3H), 1.17 (t, J=7.1 Hz, 3H). LCMS (method B) m/z 512.3 [M+H]⁺ at 3.86 min.

### 56. 5-[6-(Cyclopropylamino)pyridin-3-yl]-1-(oxan-4-yl)-N-[(3S)-9-fluoro-2-oxo-5-phenyl-1,3-dihydro-1,4-benzodiazepin-3-yl]pyrazole-4-carboxamide

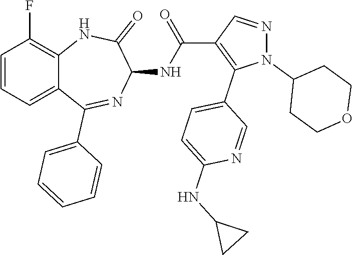 [SMILES: O=C(N[C@H]1N=C(c2ccccc2)c2cccc(F)c2NC1=O)c1cnn(C2CCOCC2)c1-c1ccc(NC2CC2)nc1]

A solution of intermediate 300 (296 mg, 0.83 mmol) and LiOH (1 M. aq., 6.64 mL, 6.64 mmol) in THF:MeOH (16 mL) was heated at 55° C. for 3 h. The reaction was cooled to rt, acidified (pH≈2) with 1 M aq. HCl and the solvent removed under reduced pressure. The residue was suspended in DMF (6 mL), then DIPEA (0.23 mL, 1.33 mmol) and HATU (278 mg, 0.732 mmol) added and the reaction stirred at rt for 10 min. (3S)-3-Amino-9-fluoro-5-phenyl-1,3-dihydro-1,4-benzodiazepin-2-one (179 mg, 0.67 mmol) was added and the reaction stirred at rt for 4 h. The mixture was poured into water (50 mL), and the resultant precipitate collected by filtration, washing with water (2×15 mL). The precipitate was dissolved in CH₂Cl₂ (50 mL), dried (Na₂SO₄), concentrated under reduced pressure and purified by flash chromatography (EtOAc) to afford a white solid (105 mg, 27%). ¹H NMR (600 MHz, DMSO-d₆) δ 10.79 (s, 1H), 8.66 (d, J=7.8 Hz, 1H), 8.33 (s, 1H), 8.02-7.93 (m, 1H), 7.60-7.49 (m, 4H), 7.51-7.41 (m, 3H), 7.32-7.25 (m, 1H), 7.17-37.09 (m, 2H), 6.65 (d, J=8.6 Hz, 1H), 5.38 (d, J=7.7 Hz, 1H), 4.20-4.11 (m, 1H), 3.95-3.85 (m, 2H), 2.18-2.06 (m, 2H), 1.81-1.72 (m, 2H), 0.742-0.62 (m, 2H), 0.48-0.33 (m, 2H). LRMS m/z: 580.1 [M+H]⁺

The following compounds of the invention were prepared by an analogous procedure to that described for the compound of Example 56.

### Compounds Prepared by Amide Coupling as in Example 56

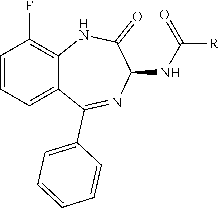

| | | | | LRMS |
|---|---|---|---|---|
| Example | R | Name | ¹H NMR (DMSO-d₆) δ | m/z |
| 57 | 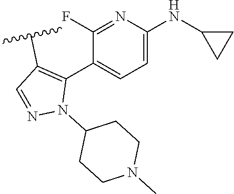 [SMILES: *c1cnn(C2CCN(C)CC2)c1-c1ccc(NC2CC2)nc1F] | 5-[6- (Cyclopropylamino)-2- fluoropyridin-3-yl]-N- [(3S)-9-fluoro-2-oxo-5- phenyl-1,3-dihydro-1,4- benzodiazepin-3-yl]-1- (1-methylpiperidin-4- yl)pyrazole-4- carboxamide | (600 MHz) 10.79 (s, 1H), 8.90 (dd, J = 19.4, 8.0 Hz, 1H), 8.42 (s, 1H), 7.60-7.43 (m, 8H), 7.31-7.24 (m, 1H), 7.14-7.10 (m, 1H), 6.52 (s, 1H), 5.40 (d, J = 7.8 Hz, 1H), 3.81-3.71 (m, 1H), 2.86-2.76 (m, 2H), 2.15 (s, 3H), 2.13-2.06 (m, 2H), 1.92-1.81 (m, 2H), 1.77-1.66 (m, 2H), 0.75- 0.68 (m, 2H), 0.50-0.42 (m, 2H). | 611.6 [M + H]⁺ |
| 58 | 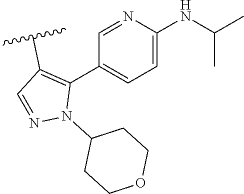 [SMILES: *c1cnn(C2CCOCC2)c1-c1ccc(NC(C)C)nc1] | 1-(Oxan-4-yl)-5-[6- (propan-2- ylamino)pyridin-3-yl]- N-[(3S)-9-fluoro-2- oxo-5-phenyl-1,3- dihydro-1,4- benzodiazepin-3- yl]pyrazole-4- carboxamide | (600 MHz) 10.79 (s, 1H), 8.62 (d, J = 7.8 Hz, 1H), 8.31 (s, 1H), 7.95 (dd, J = 2.5, 0.7 Hz, 1H), 7.61-7.51 (m, 4H), 7.50-7.42 (m, 2H), 7.34 (dd, J = 8.6, 2.4 Hz, 1H), 7.32-7.24 (m, 1H), 7.13 (d, J = 7.9 Hz, 1H), 6.73 (d, J = 7.5 Hz, 1H), 6.49 (dd, J = 8.7, 0.8 Hz, 1H), 5.38 (d, J = 7.7 Hz, 1H), 4.21-4.10 (m, 1H), 4.07-3.99 (m, 1H), 3.96-3.85 (m, 2H), 2.18-2.04 (m, 2H), 1.82-1.70 (m, 2H), 1.15 (dd, J = 6.5, 1.8 Hz, 6H). | 582.1 [M + H]⁺ |
| 59 | 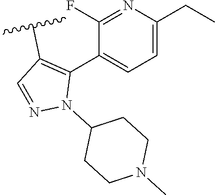 [SMILES: *c1cnn(C2CCN(C)CC2)c1-c1ccc(CC)nc1F] | 5-(4-Ethyl-2- fluorophenyl)-1-(1- methylpiperidin-4-yl)- N-[(3S)-9-fluoro-2- oxo-5-phenyl-1,3- dihydro-1,4- benzodiazepin-3- yl]pyrazole-4- carboxamide | (500 MHz) 10.78 (d, J = 5.8 Hz, 1H), 8.94 (dd, J = 11.7, 7.8 Hz, 1H), 8.47 (s, 1H), 7.68-7.38 (m, 6H), 7.38- 7.17 (m, 2H), 7.17-7.00 (m, 3H), 5.39 (d, J = 7.8 Hz, 1H), 3.78-3.61 (m, 1H), 2.82 (d, J = 11.3 Hz, 2H), 2.67 (q, J = 7.6 Hz, 2H), 2.22-2.00 (m, 5H), 1.92-1.60 (m, 4H), 1.21 (t, J = 7.6 Hz, 3H). | 583.4 [M + H]⁺ |

### Example 60: Efficacy In Vitro

Compounds were subjected to RSV plaque reduction assays according to the following protocol.

### Plaque Reduction Assay.

Hep-G2 cells (ECACC, 85011430) were passaged in flasks and seeded in 24-well plates in DMEM containing antibiotics and supplemented with 10% FBS. During inoculation and subsequent incubation, cells were cultured in DMEM containing 2% FBS. 100 plaque forming unit/well of RSV (RSV A2 ECACC, 0709161v) was mixed with eight serial dilutions of compound. Subsequently, 100 μL of the virus/compound mixtures was added to confluent Hep-G2 cell monolayers. The cells and virus/compound mixtures were incubated at 37° C. in a humidified 5% CO₂ incubator for 2 h prior to removal of the inoculum and addition of 1 mL of overlay (DMEM containing 2% FBS and 0.8% CMC) containing compound dilutions. The cells and were incubated at 37° C. in a humidified 5% CO₂ incubator for 2 days.

Cells were washed with PBS before adding 75/25% v/v EtOH/MeOH, for 3 min. Fixative was removed and plates were washed with PBS. A pre-titrated amount of the primary antibody was added in 200 μL PBS/2% milk powder, and plates incubated for 90 min at 37° C. The plates were washed 3 times with PBS/0.05% Tween20 before addition of rabbit anti-goat horse radish peroxidase in 200 μL PBS/2% milk powder, and incubated for 1 h at 37° C. Following three wash steps with PBS/0.05% Tween20, 200 μL ready-to-use TrueBlue was added and plates were incubated at rt for 10-15 min before washing with water. After removal of water, plates were air-dried in the dark.

Plates were scanned and analysed using the Immunospot S6 Macro analyser, which is equipped with BioSpot analysis software for counting immunostained plaques (virospots). Plaque counts were used to calculate % infection relative to the mean of the plaque count in the virus control wells for RSV. The EC₅₀ value was calculated as 50% reduction in signal, respectively, by interpolation of inhibition curves fitted with a 4-parameter nonlinear regression with a variable slope in Dotmatics. Plaque EC₅₀ and cell toxicity CC₅₀ values are a mean of at least two experiments and figures are rounded to whole units.

### Results

| | RSV A2 | Cell Cytotoxicity |
|---|---|---|
| Example | Plaque EC₅₀ (nM) | CC₅₀ (nM) |
| 1 | 91 | >25000 |
| 2 | 88 | >25000 |
| 3 | 97 | >25000 |
| 4 | 42 | >25000 |
| 5 | 87 | >25000 |
| 6 | 32 | >25000 |
| 7 | 69 | >25000 |
| 8 | 32 | >25000 |
| 9 | 73 | >25000 |
| 10 | 89 | >25000 |
| 11 | 62 | >25000 |
| 12 | 45 | >25000 |
| 13 | 68 | >25000 |
| 14 | 95 | >25000 |
| 15 | 51 | >25000 |
| 16 | 50 | >25000 |
| 17 | 52 | >25000 |
| 18 | 83 | >25000 |
| 19 | 37 | >25000 |
| 20 | 59 | >25000 |
| 21 | 21 | >25000 |
| 22 | 40 | >25000 |
| 23 | 50 | >25000 |
| 24 | 33 | >25000 |
| 25 | 49 | >25000 |
| 26 | 36 | >25000 |
| 27 | 57 | >25000 |
| 28 | 39 | >25000 |
| 29 | 54 | >25000 |
| 30 | 32 | >25000 |
| 31 | 55 | >25000 |
| 32 | 58 | >25000 |
| 33 | 80 | >25000 |
| 34 | 96 | >25000 |
| 35 | 60 | >25000 |
| 36 | 45 | >25000 |
| 37 | 78 | >25000 |
| 38 | 105 | >25000 |
| 39 | 62 | >25000 |
| 40 | 67 | >25000 |
| 41 | 91 | >25000 |
| 42 | 41 | >25000 |
| 43 | 48 | >25000 |
| 44 | 48 | >25000 |
| 45 | 38 | >25000 |
| 46 | 34 | >25000 |
| 47 | 30 | >25000 |
| 48 | 92 | >25000 |
| 49 | 100 | >25000 |
| 50 | 81 | >25000 |
| 51 | 53 | >7790 |
| 52 | 87 | >25000 |
| 53 | 72 | >25000 |
| 54 | 51 | >25000 |
| 55 | 33 | >25000 |
| 56 | 78 | >25000 |
| 57 | 84 | >9409 |
| 58 | 99 | >25000 |
| 59 | 107 | 15209 |

### Example 61: In Vitro Pharmacokinetics

Compounds were subjected to the following assays to investigate liver microsomal stability, permeability and plasma protein binding.

### Microsomal Incubation: Experimental Procedure

Pooled liver microsomes were purchased from a reputable commercial supplier and stored at −80° C. prior to use. Microsomes (final protein concentration 0.5 mg/mL), 0.1 M phosphate buffer pH 7.4 and test compound (final substrate concentration 1 μM; final DMSO concentration 0.25%) were pre-incubated at 37° C. prior to the addition of NADPH (final concentration 1 mM) to initiate the reaction. The final incubation volume was 50 μL. A control incubation was included for each compound tested where 0.1 M phosphate buffer pH 7.4 was added instead of NADPH (minus NADPH). Two control compounds were included with each species. All incubations were performed singularly for each test compound. Each compound was incubated for 0, 5, 15, 30 and 45 min. The control (minus NADPH) was incubated for 45 min only. The reactions were stopped by transferring incubate into acetonitrile at the appropriate time points, in a 1:3 ratio. The termination plates are centrifuged at 3,000 rpm for 20 min at 4° C. to precipitate the protein. Following protein precipitation, the sample supernatants were combined in cassettes of up to 4 compounds, internal standard added, and samples analysed by LC-MS/MS. From a plot of ln peak area ratio (compound peak area/internal standard peak area) against time, the gradient of the line was determined. Subsequently, half-life (t_{1/2}) and intrinsic clearance (CLᵢₙₜ) were calculated. Compounds with low clearance (>80% remaining at 45 min) under the assay conditions are denoted as t_{1/2}>140 min.

### Results

| Example | Liver Microsomal Stability: t_{1/2} (min); rat/dog/human |
|---|---|
| 1 | 24.4/11.1/48.1 |
| 2 | 30.3/23.9/44.4 |
| 3 | 88.8/45.3/287 |
| 4 | 68.5/14.4/52.0 |
| 5 | 47.4/45.7/50.1 |
| 6 | 31.5/72.5/23.0 |
| 7 | 595/85.8/97.0 |
| 8 | >140/104/193 |
| 9 | 141/89.1/220 |
| 10 | 20.2/85.8/48.1 |
| 11 | 76.3/24.0/82.4 |
| 12 | 51.1/68.5/193 |
| 13 | 9.6/35.6/42.9 |
| 14 | 37.9/59.3/93.7 |
| 15 | 69.8/57.7/72.3 |
| 16 | 72.7/22.2/50.6 |
| 17 | 318/68.9/120 |
| 18 | 93.5/149/144 |
| 19 | >140/90.8/154 |
| 20 | 116/325/83.1 |
| 21 | 87.6/124/170 |
| 22 | >140/88.6/287 |
| 24 | 484/198/>140 |
| 25 | 46.3/17.0/82.8 |
| 26 | 70.2/30.8/84.9 |
| 27 | 23.9/12.6/43.1 |
| 28 | 22.7/30.8/41 |
| 29 | 45.2/44.0/73.9 |
| 30 | 53.1/63.7/253 |
| 31 | 36.3/65.7/116 |
| 32 | 127/331/298 |
| 33 | 56/81/80.3 |
| 34 | 121/85.8/119 |
| 35 | 181/90.9/164 |
| 36 | 93.2/191/68.5 |
| 38 | 42.0/48.6/35.2 |
| 39 | 21.4/45.7/62.0 |
| 40 | 81.1/72.9/49.6 |
| 42 | 85.8/480/194 |
| 43 | 44.9/161/170 |
| 44 | 55.9/90/201 |
| 47 | 33.1/81.4/105 |
| 48 | 118/>140/355 |
| 49 | 73.4/110/181 |
| 50 | 44.2/163/94.3 |
| 51 | 92.8/212/171 |
| 53 | 200/183/>140 |
| 55 | 26.5/39/41.3 |
| 57 | 116/175/84.7 |

### Hepatocyte Incubation: Experimental Procedure

Cryopreserved pooled hepatocytes were purchased from a reputable commercial supplier and stored in liquid nitrogen prior to use. Williams E media supplemented with 2 mM L-glutamine and 25 mM HEPES and test compound (final substrate concentration 3 μM; final DMSO concentration 0.25%) are pre-incubated at 37° C. prior to the addition of a suspension of cryopreserved hepatocytes (final cell density 0.5×10⁶ viable cells/mL in Williams E media supplemented with 2 mM L-glutamine and 25 mM HEPES) to initiate the reaction. The final incubation volume is 500 μL. Two control compounds were included with each species, alongside appropriate vehicle control. The reactions are stopped by transferring 50 μL of incubate to 100 μL acetonitrile containing internal standard at the appropriate time points. Samples were removed at 6 time points (0, 5, 15, 30, 45 and 60 min) over the course of a 60 min experiment. The termination plates are centrifuged at 2500 rpm at 4° C. for 30 min to precipitate the protein. Following protein precipitation, the sample supernatants were combined in cassettes of up to 4 compounds and analysed using generic LC-MS/MS conditions. From a plot of ln peak area ratio (compound peak area/internal standard peak area) against time, the gradient of the line is determined. Subsequently, half-life (t_{1/2}) and intrinsic clearance (CLᵢₙₜ) were calculated. Compounds with low clearance (>80% remaining at 60 min) under the assay conditions are denoted as t_{1/2}>186 min.

### Results

| Example | Liver Hepatocyte Stability: t_{1/2} (min); rat/dog/human |
|---|---|
| 3 | 73.8/67.4/52.6 |
| 4 | 47.3/42.1/53.5 |
| 11 | 151/48.2/194 |
| 19 | 185/72.2/>186 |
| 20 | 115/>186/190 |
| 21 | 284/>186/>186 |
| 22 | 120/90.7/207 |
| 23 | 96.7/127/>186 |
| 24 | 228/139/162 |
| 25 | >186/68.1/506 |
| 26 | >186/160/>186 |
| 30 | 98.5/>186/71.3 |
| 32 | 376/>186/>186 |
| 42 | 72.3/100/421 |

### Example 62: In Vivo Pharmacokinetics

The pharmacokinetics of compounds were studied in vivo in rats at doses of 1 mg/kg (IV) and 10 mg/kg (PO).

### Rat Pharmacokinetics

### Methods

Male rats (Sprague Dawley) surgically prepared with a jugular vein cannula were treated with experimental compounds via intravenous administration (IV; n=3; 1 mg/kg) or oral administration (PO; n=3; 10 mg/kg). Compounds were formulated as a solution in 40:60 dimethylacetamide:saline (IV administration), a suspension in 1% methyl cellulose (viscosity: 15 cP), 0.1% Tween80 in water (PO administration: Example 3), a solution in 10% DMSO/10% Cremaphor/80% water (PO administration: Examples 4, 11, 21, 23, 25, 26, 32) or a solution in 10% DMSO/20% Cremaphor/70% water (PO administration: Examples 19, 20, 22, 24, 30). Animals were observed for any overt clinical signs or symptoms. Serial blood samples were collected via the cannula at 0.02, 0.08, 0.25, 0.5, 1, 2, 4, 6, 8 and 24 h post IV dosing of compound, and at 0.08, 0.25, 0.5, 1, 2, 4, 6, 8 and 24 h post oral dosing of compound, and plasma was prepared by centrifugation and stored immediately at −80° C. Samples were subsequently thawed, prepared for analysis by protein precipitation with acetonitrile, and analysed by tandem LCMS using electrospray ionisation using a matrix-matched calibration curve. PK parameters were calculated from the resulting data.

### Results

| Example | 3 | 4 | 11 | 19 | 20 | 21 | 22 |
|---|---|---|---|---|---|---|---|
| PO AUCₗₐₛₜ | 2072 | 9022 | 11220 | 7293 | 1166 | 8185 | 14896 |
| (hr*ng/mL) | | | | | | | |
| Cl (mL/min/kg) | 13.3 | 22.7 | 13.9 | 14.3 | 32.9 | 18.6 | 10 |
| V_{d} (L/kg) | 2.4 | 2.0 | 2.0 | 1.6 | 2.9 | 2.1 | 1.9 |
| Cₘₐₓ (ng/mL) | 199 | 1344 | 984 | 1325 | 258 | 1587 | 1442 |
| C 8 h (ng/mL) | 108 | 470 | 673 | 315 | 25.7 | 385 | 734 |
| IV t½ (h) | 2.2 | 1.3 | 3.2 | 1.7 | 1.5 | 1.7 | 2.4 |
| PO t½ (h) | 4.6 | 2.2 | 2.2 | 3.0 | 1.4 | 3.2 | 2.6 |
| F (%) | 17.1% | 124.0% | 93.0% | 64.6% | 23.9% | 98.2% | 88.3% |

| Example | 23 | 24 | 25 | 26 | 30 | 32 |
|---|---|---|---|---|---|---|
| PO AUCₗₐₛₜ | 4626 | 5539 | 7470 | 19394 | 8538 | 1939 |
| (hr*ng/mL) | | | | | | |
| Cl (mL/min/kg) | 12.2 | 15 | 15.9 | 8.2 | 11.2 | 17.7 |
| V_{d} (L/kg) | 1.7 | 1.4 | 0.9 | 1.1 | 1.4 | 1.1 |
| Cₘₐₓ (ng/mL) | 930 | 791 | 1150 | 2116 | 918 | 452 |
| C 8 h (ng/mL) | 146 | 182 | 294 | 919 | 444 | 84 |
| IV t½ (h) | 2.2 | 1.3 | 1.3 | 2.6 | 1.8 | 1.2 |
| PO t½ (h) | 3.0 | 3.4 | 2.7 | 2.6 | 3.0 | 5.2 |
| F (%) | 34.7% | 50.0% | 71.5% | 95.6% | 57.0% | 19.3% |

### Dog Pharmacokinetics

The pharmacokinetics of compounds of the invention were studied in vivo in dogs.

### Methods

Male Beagle dogs were treated with experimental compounds via intravenous administration (n=2; 0.5 mg/kg) or oral administration (n=2; 3 mg/kg or 4 mg/kg). Compounds were formulated as a solution in 20% dimethylacetamide/80% (2-hydroxypropyl)-β-cyclodextrin (20% w/v) (IV administration) or a solution in 10% dimethylacetamide/90% (2-hydroxypropyl)-β-cyclodextrin (20% w/v) (PO administration). Animals were observed for any overt clinical signs or symptoms. Serial blood samples were collected from the jugular vein at 0.03, 0.08, 0.25, 0.5, 1, 2, 4, 6, 8 and 24 h post IV dosing of compound, and at 0.08, 0.25, 0.5, 1, 2, 4, 6, 8 and 24 h post oral dosing of compound, and plasma was prepared by centrifugation and stored immediately at −80° C. Samples were subsequently thawed, prepared for analysis by protein precipitation with acetonitrile, and analysed by tandem LCMS using electrospray ionisation using a matrix-matched calibration curve. PK parameters were calculated from the resulting data.

### Results

| Example | 4 | 21 | 22 | 24 |
|---|---|---|---|---|
| Dose: IV/PO (mg/kg) | 0.5/4 | 0.5/3 | 0.5/4 | 0.5/4 |
| PO AUCₗₐₛₜ (hr*ng/mL) | 2679 | 3608 | 6052 | 2302 |
| Cl (mL/min/kg) | 10.3 | 4.2 | 4.1 | 6.5 |
| V_{d} (L/kg) | 0.79 | 1.3 | 1.4 | 0.86 |
| Cₘₐₓ (ng/mL) | 780 | 500 | 692 | 450 |
| C 8 h (ng/mL) | 56.1 | 146 | 281 | 67 |
| IV t_{1/2} (h) | 1 | 7.6 | 4.9 | 1.5 |
| PO t_{1/2} (h) | 2.3 | 5.6 | 6.8 | 2.4 |
| F (%) | 41.5% | 33.6% | 40.1% | 22.7% |

### Example 63: Aqueous Formulation

The compound of Example 1 is formulated as a solution in 30% w/v captisol (i.e. sulfobutylether-beta-cyclodextrin) at pH4 according to the following procedure.

A carrier of 30% w/v captisol (i.e. sulfobutylether-beta-cyclodextrin) is prepared by weighing the required amount of captisol into a suitable vessel, adding approximately 80% of the final volume of water and magnetically stirring until a solution is formed. The carrier is then made up to volume with water.

An aqueous solution of a compound of Example 1 is prepared by weighing 175 mg of the compound into a suitable vessel and adding approximately 80% of the required volume of the carrier. Using an aqueous solution of hydrochloric acid, the pH is adjusted to pH2 and the resulting mixture is magnetically stirred until a solution is formed. The formulation is then made up to volume with carrier and the pH is adjusted to pH4 using an aqueous solution of sodium hydroxide.

### Example 64: Tablet Composition

Tablets, each weighing 0.15 g and containing 25 mg of a compound of the invention are manufactured as follows:

### Composition for 10,000 Tablets

Compound of the invention (250 g)

### Lactose (800 g)

Corn starch (415 g)
Talc powder (30 g)
Magnesium stearate (5 g)

The compound of the invention, lactose and half of the corn starch are mixed. The mixture is then forced through a sieve 0.5 mm mesh size. Corn starch (10 g) is suspended in warm water (90 mL). The resulting paste is used to granulate the powder. The granulate is dried and broken up into small fragments on a sieve of 1.4 mm mesh size. The remaining quantity of starch, talc and magnesium is added, carefully mixed and processed into tablets.

### Example 65: Injectable Formulation

| Compound of the invention | 200 | mg |
|---|---|---|
| Hydrochloric Acid Solution 0.1M or | 4.0 to 7.0 | |
| Sodium Hydroxide Solution 0.1M q.s. to pH | | |
| Sterile water q.s. to | 10 | mL |

The compound of the invention is dissolved in most of the water (35° C.-40° C.) and the pH adjusted to between 4.0 and 7.0 with the hydrochloric acid or the sodium hydroxide as appropriate. The batch is then made up to volume with water and filtered through a sterile micropore filter into a sterile 10 mL amber glass vial (type 1) and sealed with sterile closures and overseals.

### Example 66: Intramuscular Injection

| | | |
|---|---|---|
| Compound of the invention | 200 | mg |
| Benzyl Alcohol | 0.10 | g |
| Glycofurol 75 | 1.45 | g |
| Water for injection q.s to | 3.00 | mL |

The compound of the invention is dissolved in the glycofurol. The benzyl alcohol is then added and dissolved, and water added to 3 mL. The mixture is then filtered through a sterile micropore filter and sealed in sterile 3 mL glass vials (type 1).

### Example 67: Syrup Formulation

| | | |
|---|---|---|
| Compound of invention | 250 | mg |
| Sorbitol Solution | 1.50 | g |
| Glycerol | 2.00 | g |
| Sodium benzoate | 0.005 | g |
| Flavour | 0.0125 | mL |
| Purified Water q.s. to | 5.00 | mL |

The compound of the invention is dissolved in a mixture of the glycerol and most of the purified water. An aqueous solution of the sodium benzoate is then added to the solution, followed by addition of the sorbital solution and finally the flavour. The volume is made up with purified water and mixed well.

We claim:

1. A compound which is a benzodiazepinyl pyrazole of formula (I): 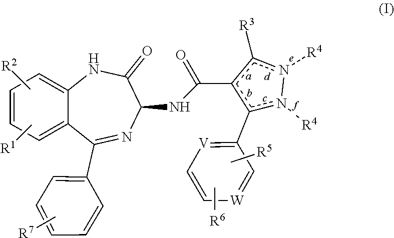
wherein:
each of R¹ and R² is independently H or halo;
R³ is H, C₁-C₆ alkyl, —NHR⁸ or —OR′;
either (i) [symbol] , [symbol] and [symbol] are all bonds, with [symbol] , [symbol] and [symbol] absent; or [symbol] , [symbol] and [symbol] are all bonds, with [symbol] , [symbol] and [symbol] absent;
R⁴ is H or a group selected from C₁-C₆ alkyl, C₃-C₆ cycloalkyl and 4- to 10-membered heterocyclyl, the group being unsubstituted or substituted;
R⁵ is H or halo;
R⁶ is —OR⁸, —NR⁸R⁹ or —R⁸;
R⁷ is H or halo;
each of R⁸ and R⁹ is independently H or a group selected from C₁-C₆ alkyl, C₃-C₆ cycloalkyl and 4- to 10-membered heterocyclyl, the group being unsubstituted or substituted;
R′ is H or C₁-C₆ alkyl; and
one of V and W is CH and the other is N or CH;
or a pharmaceutically acceptable salt thereof.

2. A compound according to claim 1, wherein R² is F at the 9-position of the benzodiazepinyl ring system.

3. A compound according to claim 1 wherein [symbol] , [symbol] and [symbol] are all bonds, with [symbol] , [symbol] and [symbol] absent.

4. A compound according to claim 1 wherein [symbol] , [symbol] and [symbol] are all bonds, with [symbol] , [symbol] and [symbol] absent.

5. A compound according to claim 1 wherein V is N and W is CH; or V is CH and W is N.

6. A compound according to claim 1 wherein R² is a halo substituent at the 9-position of the benzodiazepinyl ring system.

7. A compound according to claim 1 wherein R⁵ is at ring position 2 and R⁶ is at ring position 4 of the six-membered ring to which they are both bonded.

8. A compound according to claim 1 which is selected from:
1-Ethyl-5-[2-fluoro-6-(methylamino)pyridin-3-yl]-N-[(3S)-2-oxo-5-phenyl-1,3-dihydro-1,4-benzodiazepin-3-yl]pyrazole-4-carboxamide;
1-Ethyl-5-[6-(ethylamino)-2-fluoropyridin-3-yl]-N-[(3S)-2-oxo-5-phenyl-1,3-dihydro-1,4-benzodiazepin-3-yl]pyrazole-4-carboxamide;
1-Ethyl-5-[2-fluoro-6-(propan-2-ylamino)pyridin-3-yl]-N-[(3S)-2-oxo-5-phenyl-1,3-dihydro-1,4-benzodiazepin-3-yl]pyrazole-4-carboxamide;
1-Ethyl-5-[2-fluoro-6-(cyclopropylamino)pyridin-3-yl]-N-[(3S)-2-oxo-5-phenyl-1,3-dihydro-1,4-benzodiazepin-3-yl]pyrazole-4-carboxamide;
5-[6-(Cyclobutylamino)-2-fluoropyridin-3-yl]-1-ethyl-N-[(3S)-2-oxo-5-phenyl-1,3-dihydro-1,4-benzodiazepin-3-yl]pyrazole-4-carboxamide;
1-Ethyl-5-[2-fluoro-6-[(1-methylcyclopropyl)amino]pyridin-3-yl]-N-[(3S)-2-oxo-5-phenyl-1,3-dihydro-1,4-benzodiazepin-3-yl]pyrazole-4-carboxamide;
5-[5-(Cyclopropylamino)-3-fluoropyridin-2-yl]-1-ethyl-N-[(3S)-2-oxo-5-phenyl-1,3-dihydro-1,4-benzodiazepin-3-yl]pyrazole-4-carboxamide;
1-Ethyl-5-[3-fluoro-5-(propan-2-ylamino)pyridin-2-yl]-N-[(3S)-2-oxo-5-phenyl-1,3-dihydro-1,4-benzodiazepin-3-yl]pyrazole-4-carboxamide;
1-Ethyl-5-[2-fluoro-4-(propan-2-ylamino)phenyl]-N-[(3S)-2-oxo-5-phenyl-1,3-dihydro-1,4-benzodiazepin-3-yl]pyrazole-4-carboxamide;
1-Ethyl-5-[6-(ethylamino)-2-fluoropyridin-3-yl]-3-methyl-N-[(3S)-2-oxo-5-phenyl-1,3-dihydro-1,4-benzodiazepin-3-yl]pyrazole-4-carboxamide;
5-[6-(Cyclopropylamino)-2-fluoropyridin-3-yl]-1-ethyl-N-[(3S)-9-fluoro-2-oxo-5-phenyl-1,3-dihydro-1,4-benzodiazepin-3-yl]pyrazole-4-carboxamide;
5-[5-(Cyclopropylamino)-3-fluoropyridin-2-yl]-1-ethyl-N-[(3S)-9-fluoro-2-oxo-5-phenyl-1,3-dihydro-1,4-benzodiazepin-3-yl]pyrazole-4-carboxamide;
1-Ethyl-5-[5-(ethylamino)-3-fluoropyridin-2-yl]-N-[(3S)-9-fluoro-2-oxo-5-phenyl-1,3-dihydro-1,4-benzodiazepin-3-yl]pyrazole-4-carboxamide;
1-cyclopropyl-5-[6-(ethylamino)-2-fluoropyridin-3-yl]-N-[(3S)-2-oxo-5-phenyl-1,3-dihydro-1,4-benzodiazepin-3-yl]pyrazole-4-carboxamide;
1-cyclopropyl-5-[6-(cyclopropylamino)-2-fluoropyridin-3-yl]-N-[(3S)-2-oxo-5-phenyl-1,3-dihydro-1,4-benzodiazepin-3-yl]pyrazole-4-carboxamide;
5-[6-(cyclopropylamino)-2-fluoropyridin-3-yl]-N-[(3S)-2-oxo-5-phenyl-1,3-dihydro-1,4-benzodiazepin-3-yl]-1-propan-2-ylpyrazole-4-carboxamide;
1-Ethyl-N-[(3S)-9-fluoro-2-oxo-5-phenyl-1,3-dihydro-1,4-benzodiazepin-3-yl]-5-[3-fluoro-5-(propan-2-ylamino)pyridin-2-yl]pyrazole-4-carboxamide;
5-[6-(Cyclopropylamino)-2-fluoropyridin-3-yl]-1-(oxetan-3-yl)-N-[(3S)-2-oxo-5-phenyl-1,3-dihydro-1,4-benzodiazepin-3-yl]pyrazole-4-carboxamide;
5-[6-(Cyclopropylamino)-2-fluoropyridin-3-yl]-1-(oxetan-3-yl)-N-[(3S)-9-fluoro-2-oxo-5-phenyl-1,3-dihydro-1,4-benzodiazepin-3-yl]pyrazole-4-carboxamide;
5-[6-(Cyclopropylamino)-2-fluoropyridin-3-yl]-N-[(3S)-2-oxo-5-phenyl-1,3-dihydro-1,4-benzodiazepin-3-yl]-1-(oxan-4-yl)pyrazole-4-carboxamide;
5-[6-(Cyclopropylamino)-2-fluoropyridin-3-yl]-N-[(3S)-9-fluoro-2-oxo-5-phenyl-1,3-dihydro-1,4-benzodiazepin-3-yl]-1-(oxan-4-yl)pyrazole-4-carboxamide;
N-[(3S)-9-fluoro-2-oxo-5-phenyl-1,3-dihydro-1,4-benzodiazepin-3-yl]-5-[2-fluoro-6-(propan-2-ylamino)pyridin-3-yl]-1-(oxan-4-yl)pyrazole-4-carboxamide;
5-[2-Fluoro-6-(propan-2-ylamino)pyridin-3-yl]-1-(oxan-4-yl)-N-[(3S)-2-oxo-5-phenyl-1,3-dihydro-1,4-benzodiazepin-3-yl]pyrazole-4-carboxamide;
5-[6-(Ethylamino)-2-fluoropyridin-3-yl]-N-[(3S)-9-fluoro-2-oxo-5-phenyl-1,3-dihydro-1,4-benzodiazepin-3-yl]-1-(oxan-4-yl)pyrazole-4-carboxamide;
3-[6-(Cyclopropylamino)-2-fluoropyridin-3-yl]-1-ethyl-N-[(3S)-2-oxo-5-phenyl-1,3-dihydro-1,4-benzodiazepin-3-yl]pyrazole-4-carboxamide;
3-[6-(Cyclopropylamino)-2-fluoropyridin-3-yl]-1-ethyl-N-[(3S)-9-fluoro-2-oxo-5-phenyl-1,3-dihydro-1,4-benzodiazepin-3-yl]pyrazole-4-carboxamide;
1-Ethyl-3-[2-fluoro-6-(propylamino)pyridin-3-yl]-N-[(3S)-2-oxo-5-phenyl-1,3-dihydro-1,4-benzodiazepin-3-yl]pyrazole-4-carboxamide;
1-Ethyl-N-[(3S)-9-fluoro-2-oxo-5-phenyl-1,3-dihydro-1,4-benzodiazepin-3-yl]-3-[2-fluoro-6-(propylamino)pyridin-3-yl]pyrazole-4-carboxamide;
1-tert-butyl-3-[6-(cyclopropylamino)-2-fluoropyridin-3-yl]-N-[(3S)-2-oxo-5-phenyl-1,3-dihydro-1,4-benzodiazepin-3-yl]pyrazole-4-carboxamide;
1-tert-butyl-3-[6-(cyclopropylamino)-2-fluoropyridin-3-yl]-N-[(3S)-9-fluoro-2-oxo-5-phenyl-1,3-dihydro-1,4-benzodiazepin-3-yl]pyrazole-4-carboxamide;
3-[6-(Cyclopropylamino)-2-fluoropyridin-3-yl]-1-(oxan-4-yl)-N-[(3S)-2-oxo-5-phenyl-1,3-dihydro-1,4-benzodiazepin-3-yl]pyrazole-4-carboxamide;
3-[6-(Cyclopropylamino)-2-fluoropyridin-3-yl]-N-[(3S)-9-fluoro-2-oxo-5-phenyl-2,3-dihydro-1H-1,4-benzodiazepin-3-yl]-1-(oxan-4-yl)-1H-pyrazole-4-carboxamide;
3-[6-(Cyclopropylamino)-2-fluoropyridin-3-yl]-N-[(3S)-9-fluoro-2-oxo-5-phenyl-2,3-dihydro-1H-1,4-benzodiazepin-3-yl]-1-(2,2,2-trifluoroethyl)-1H-pyrazole-4-carboxamide;
(3-[6-(Cyclopropylamino)-2-fluoropyridin-3-yl]-1-(4,4-difluorocyclohexyl)-N-[(3S)-2-oxo-5-phenyl-1,3-dihydro-1,4-benzodiazepin-3-yl]pyrazole-4-carboxamide;
3-[6-(Cyclopropylamino)-2-fluoropyridin-3-yl]-1-(4,4-difluorocyclohexyl)-N-[(3S)-9-fluoro-2-oxo-5-phenyl-1,3-dihydro-1,4-benzodiazepin-3-yl]pyrazole-4-carboxamide;
3-[6-(Cyclopropylamino)-2-fluoropyridin-3-yl]-N-[(3S)-9-fluoro-2-oxo-5-phenyl-1,3-dihydro-1,4-benzodiazepin-3-yl]-1-(oxetan-3-yl)pyrazole-4-carboxamide;
3-[6-(Cyclopropylamino)-2-fluoropyridin-3-yl]-1-(oxetan-3-yl)-N-[(3S)-2-oxo-5-phenyl-1,3-dihydro-1,4-benzodiazepin-3-yl]pyrazole-4-carboxamide;
1-Ethyl-5-(3-fluoro-5-(methylamino)pyridin-2-yl)-N—((S)-2-oxo-5-phenyl-2,3-dihydro-1H-benzo[e][1,4]diazepin-3-yl)-1H-pyrazole-4-carboxamide;
1-Ethyl-5-(5-(ethylamino)-3-fluoropyridin-2-yl)-N—((S)-2-oxo-5-phenyl-2,3-dihydro-1H-benzo[e][1,4]diazepin-3-yl)-1H-pyrazole-4-carboxamide;
5-[6-(Ethylamino)-2-fluoropyridin-3-yl]-N-[(3S)-9-fluoro-2-oxo-5-phenyl-1,3-dihydro-1,4-benzodiazepin-3-yl]-1-(oxetan-3-yl)pyrazole-4-carboxamide;
N-[(3S)-9-Fluoro-2-oxo-5-phenyl-1,3-dihydro-1,4-benzodiazepin-3-yl]-5-[2-fluoro-6-(propan-2-ylamino)pyridin-3-yl]-1-(oxetan-3-yl)pyrazole-4-carboxamide;
5-[6-(Ethylamino)-2-fluoropyridin-3-yl]-1-(oxan-4-yl)-N-[(3S)-2-oxo-5-phenyl-1,3-dihydro-1,4-benzodiazepin-3-yl]pyrazole-4-carboxamide;
5-(4-Cyclopropyl-2-fluorophenyl)-1-(oxan-4-yl)-N-[(3S)-2-oxo-5-phenyl-1,3-dihydro-1,4-benzodiazepin-3-yl]pyrazole-4-carboxamide;
5-(4-Cyclopropyl-2-fluorophenyl)-N-[(3S)-9-fluoro-2-oxo-5-phenyl-1,3-dihydro-1,4-benzodiazepin-3-yl]-1-(oxan-4-yl)pyrazole-4-carboxamide;
5-(2-Fluoro-4-propan-2-ylphenyl)-1-(oxan-4-yl)-N-[(3S)-9-fluoro-2-oxo-5-phenyl-1,3-dihydro-1,4-benzodiazepin-3-yl]pyrazole-4-carboxamide;
5-(4-Ethyl-2-fluorophenyl)-1-(oxan-4-yl)-N-[(3S)-2-oxo-5-phenyl-1,3-dihydro-1,4-benzodiazepin-3-yl]pyrazole-4-carboxamide;
5-(4-Ethyl-2-fluorophenyl)-1-(oxan-4-yl)-N-[(3S)-9-fluoro-2-oxo-5-phenyl-1,3-dihydro-1,4-benzodiazepin-3-yl]pyrazole-4-carboxamide;
5-(5-Cyclopropyl-2-fluoropyridin-3-yl)-1-ethyl-N-[(3S)-9-fluoro-2-oxo-5-phenyl-1,3-dihydro-1,4-benzodiazepin-3-yl]pyrazole-4-carboxamide;
5-(6-Cyclopropyl-2-fluoropyridin-3-yl)-N-[(3S)-9-fluoro-2-oxo-5-phenyl-1,3-dihydro-1,4-benzodiazepin-3-yl]-1-(oxan-4-yl)pyrazole-4-carboxamide;
5-(5-Cyclopropyl-2-fluoropyridin-3-yl)-1-(oxan-4-yl)-N-[(3S)-2-oxo-5-phenyl-1,3-dihydro-1,4-benzodiazepin-3-yl]pyrazole-4-carboxamide;
5-(5-Cyclopropyl-2-fluoropyridin-3-yl)-N-[(3S)-9-fluoro-2-oxo-5-phenyl-1,3-dihydro-1,4-benzodiazepin-3-yl]-1-(oxan-4-yl)pyrazole-4-carboxamide;
5-(6-Ethyl-2-fluoropyridin-3-yl)-1-(oxan-4-yl)-N-[(3S)-2-oxo-5-phenyl-1,3-dihydro-1,4-benzodiazepin-3-yl]pyrazole-4-carboxamide;
5-(6-Ethyl-2-fluoropyridin-3-yl)-1-(oxan-4-yl)-N-[(3S)-9-fluoro-2-oxo-5-phenyl-1,3-dihydro-1,4-benzodiazepin-3-yl]pyrazole-4-carboxamide;
3-Ethoxy-1-ethyl-5-(2-fluorophenyl)-N-[(3S)-2-oxo-5-phenyl-1,3-dihydro-1,4-benzodiazepin-3-yl]pyrazole-4-carboxamide;
3-Ethoxy-1-ethyl-N-[(3 S)-9-fluoro-2-oxo-5-phenyl-1,3-dihydro-1,4-benzodiazepin-3-yl]-5-(2-fluorophenyl)pyrazole-4-carboxamide;
5-[6-(Cyclopropylamino)pyridin-3-yl]-1-(oxan-4-yl)-N-[(3S)-9-fluoro-2-oxo-5-phenyl-1,3-dihydro-1,4-benzodiazepin-3-yl]pyrazole-4-carboxamide;
5-[6-(Cyclopropylamino)-2-fluoropyridin-3-yl]-N-[(3S)-9-fluoro-2-oxo-5-phenyl-1,3-dihydro-1,4-benzodiazepin-3-yl]-1-(1-methylpiperidin-4-yl)pyrazole-4-carboxamide;
1-(Oxan-4-yl)-5-[6-(propan-2-ylamino)pyridin-3-yl]-N-[(3S)-9-fluoro-2-oxo-5-phenyl-1,3-dihydro-1,4-benzodiazepin-3-yl]pyrazole-4-carboxamide;
5-(4-Ethyl-2-fluorophenyl)-1-(1-methylpiperidin-4-yl)-N-[(3S)-9-fluoro-2-oxo-5-phenyl-1,3-dihydro-1,4-benzodiazepin-3-yl]pyrazole-4-carboxamide;
and the pharmaceutically acceptable salts thereof.

9. A pharmaceutical composition which comprises a compound as defined in claim 1 and a pharmaceutically acceptable carrier or diluent.

10.-12. (canceled)

13. A method for treating a subject suffering from or susceptible to an RSV infection, which method comprises administering to said subject an effective amount of a compound as defined in claim 1.

14.-15. (canceled)

16. A pharmaceutical composition which comprises (a) a compound as defined in claim 1, and (b) one or more therapeutic agents, together with a pharmaceutically acceptable carrier or diluent, wherein the further therapeutic agent is selected from the group consisting of:
(i) a RSV nucleocapsid(N)-protein inhibitor;
(ii) a protein inhibitor, such as one that inhibits the phosphoprotein (P) protein and/or large (L) protein;
(iii) an anti-RSV monoclonal antibody, such as an F-protein antibody;
(iv) an immunomodulating toll-like receptor compound;
(v) a respiratory virus anti-viral, such as an anti-influenza and/or anti-rhinovirus compound; and
(vi) an anti-inflammatory compound.

17. A process for producing a pharmaceutically acceptable salt as defined in claim 1, which process comprises treating a benzodiazepine derivative of formula (I) as defined in claim 1 with a suitable acid in a suitable solvent.

18. A process according to claim 16, wherein the acid is selected from hydrochloric acid, hydrobromic acid, hydroiodic acid, sulphuric acid, nitric acid, phosphoric acid, methanesulfonic acid, benzenesulphonic acid, formic acid, acetic acid, trifluoroacetic acid, propionic acid, oxalic acid, malonic acid, succinic acid, fumaric acid, maleic acid, lactic acid, malic acid, tartaric acid, citric acid, ethanesulfonic acid, aspartic acid and glutamic acid.

19. A method according to claim 13, which method further comprises administering to the subject a further therapeutic agent selected from the group consisting of:
(i) a RSV nucleocapsid(N)-protein inhibitor;
(ii) a protein inhibitor, such as one that inhibits the phosphoprotein (P) protein and/or large (L) protein;
(iii) an anti-RSV monoclonal antibody, such as an F-protein antibody;
(iv) an immunomodulating toll-like receptor compound;
(v) a respiratory virus anti-viral, such as an anti-influenza and/or anti-rhinovirus compound; and
(vi) an anti-inflammatory compound;
wherein the benzodiazepinyl pyrazole of formula (I): 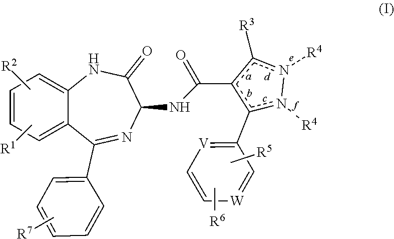
wherein:
each of R¹ and R² is independently H or halo;
R³ is H, C₁-C₆ alkyl, —NHR⁸ or —OR′;
either (i) [symbol] , [symbol] and [symbol] are all bonds, with [symbol] , [symbol] and [symbol] absent; or [symbol] , [symbol] and [symbol] are all bonds, with [symbol] , [symbol] and [symbol] absent;
R⁴ is H or a group selected from C₁-C₆ alkyl, C₃-C₆ cycloalkyl and 4- to 10-membered heterocyclyl, the group being unsubstituted or substituted;
R⁵ is H or halo;
R⁶ is —OW, —NR⁸R⁹ or —R⁸;
R⁷ is H or halo;
each of R⁸ and R⁹ is independently H or a group selected from C₁-C₆ alkyl, C₃-C₆ cycloalkyl and 4- to 10-membered heterocyclyl, the group being unsubstituted or substituted;
R′ is H or C₁-C₆ alkyl; and
one of V and W is CH and the other is N or CH;
or a pharmaceutically acceptable salt thereof; and the further therapeutic agent are administered simultaneously, separately or sequentially.
